(19) United States
(12) Patent Application Publication
(10) Pub. No.: US 2026/0224717 A1
(43) Pub. Date: Aug. 6, 2026

(54) REVERSIBLE COVALENT PROTEIN-BINDING MOLECULES, METHODS OF MAKING AND USES THEREOF

(71) Applicant: McMaster University, Hamilton (CA)

(72) Inventors: Anthony Rullo, Hamilton (CA); Tomas Frankovich, Markham (CA); Jonathan Bramson, Oakville (CA); Nickolas Serniuk, Oakville (CA)

(21) Appl. No.: 19/421,800

(22) Filed: Dec. 16, 2025

Related U.S. Application Data
(60) Provisional application No. 63/734,470, filed on Dec. 16, 2024.

(51) Int. Cl. A61K 47/64; A61K 47/54; A61K 47/55; A61P 35/00

(52) U.S. Cl. CPC A61K 47/64; A61K 47/545; A61K 47/55; A61P 35/00

(57) ABSTRACT

This application is directed to proximity inducing molecules (PIMs). More particularly, the present application relates to protein-binding molecules in combination with covalent labeling domains that induce the formation of stable ternary complexes via reversible covalent bonds, and methods of making and uses thereof. Included are compounds of Formula (I), Formula (II) or Formula (III) or a pharmaceutically acceptable salt and/or solvate thereof, and compositions comprising the same.

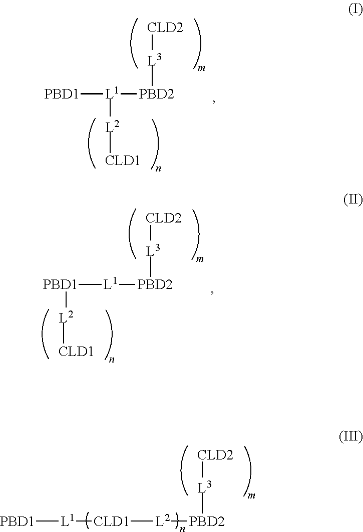

### CROSS-REFERENCE TO RELATED APPLICATION

The present application claims the benefit of priority from U.S. provisional application No. 63/734,470 filed on Dec. 16, 2024, the contents of which are incorporated herein by reference in their entirety.

### INCORPORATION OF SEQUENCE LISTING

A computer readable form of the Sequence Listing “P93249845US02_96640729_SequenceListing.xml” (20,480 bytes), filed herewith by electronic submission and created on Dec. 16, 2025, is herein incorporated by reference.

### FIELD

The present application relates to proximity inducing molecules (PIMs). More particularly, the present application relates to protein-binding molecules that induce the formation of stable ternary complexes via reversible covalent bonds, and methods of making and uses thereof.

### BACKGROUND

Proximity inducing molecules (PIMs) represent a growing class of novel therapeutic modalities, with special utility across a diverse range of disease indications.^{1,2} PIMs are small bispecific molecules that use “affinity labeling” covalent chemistry to link proteins. PIMs generally increase the effective molarity between two proteins in a ternary complex to enact a biological response.

U.S. patent publication No. 2021/0276981 describes CIRs that chemically link a targeting domain to an antibody molecule, such as an IgG, whereby the targeted antibody can be used to direct immune cells to target domains on cancer cells.

U.S. patent publication No. 2024/0083971 describes CIRs that chemically link a targeting domain to an immune cell, such as an Fc receptor-expressing macrophage, whereby the targeted immune cell can be directed to target domains on cancer cells.

There is a need to develop new proximity inducing molecules.

The background herein is included solely to explain the context of the application. This is not to be taken as an admission that any of the material referred to was published, known, or part of the common general knowledge as of the priority date.

### SUMMARY

A molecular proximity-inducing strategy to reversibly covalently crosslink two proteins within a ternary complex has been developed and is disclosed herein. The covalent proximity inducing molecules of the present application induced proteins proximity in three distinct tumor immunotherapeutic model systems, leading to significant functional enhancements. Collectively, this underscores the utility of reversible proximal covalent labeling to stabilize and enforce protein-protein interactions.

Accordingly, the present application includes a compound of Formula (I), Formula (II) or Formula (III) or a pharmaceutically acceptable salt and/or solvate thereof, wherein PBD1 and PBD2 are each independently a protein binding domain (PBD) and CLD1 and CLD2 are each independently a covalent labeling domain (CLD), and L¹, L² and L³ are each independently a linker group;
n=0 or 1 and m=0 or 1, wherein at least one of n and m is 1,
wherein PBD1 selectively binds to a first target protein and PBD2 selectively binds to a second target protein, and wherein CLD1, if present, comprises a functional group that, upon binding of PBD1 to the first target protein, forms an reversible covalent bond with a nucleophilic group in the first target protein and CLD2, if present, comprises a functional group that, upon binding of PBD2 to the second target protein, forms an reversible covalent bond with a nucleophilic group in the second target protein.

Also included is a composition comprising a compound of Formula (I), Formula (II) or Formula (III), or a pharmaceutically acceptable salt and/or solvate thereof, and at least one carrier, diluent and/or excipient.

The present application also includes a method for forming ternary protein complexes, either in a biological sample or in a subject, comprising administering an effective amount of a compound of Formula (I), Formula (II) or Formula (III), or a pharmaceutically acceptable salt and/or solvate thereof, or a composition comprising an effective amount of a compound of Formula (I), Formula (II) or Formula (III), or a pharmaceutically acceptable salt and/or solvate thereof, to the biological sample or subject.

The present application further includes a method for recruiting an antibody or an immune cell for immunotherapy, either in a biological sample or in a subject, comprising administering an effective amount of a compound of Formula (I), Formula (II) or Formula (III), or a pharmaceutically acceptable salt and/or solvate thereof, or a composition comprising an effective amount of a compound of Formula (I), Formula (II) or Formula (III), or a pharmaceutically acceptable salt and/or solvate thereof, to the biological sample or subject.

Also included is a method for recruiting an antibody or an immune cell and targeting a cell for provoking an immune response to the cell, either in a biological sample or in a subject, comprising administering an effective amount of a compound of Formula (I), Formula (II) or Formula (III), or a pharmaceutically acceptable salt and/or solvate thereof, or a composition comprising an effective amount of a compound of Formula (I), Formula (II) or Formula (III), or a pharmaceutically acceptable and/or solvate thereof, to the biological sample or the subject.

The present application also includes a method for binding tumor antigens on a cell, either in a biological sample or in a subject, comprising administering an effective amount of a compound of Formula (I), Formula (II) or Formula (III), or a pharmaceutically acceptable salt and/or solvate thereof, or a composition comprising an effective amount of a compound of Formula (I), Formula (II) or Formula (III), or a pharmaceutically acceptable salt and/or solvate thereof, to the biological sample or the subject.

Further included is a method of treating a disease, disorder, or condition that is treatable by engaging an immune response, comprising administering a therapeutically effective amount of a compound of Formula (I), Formula (II) or Formula (III), or a pharmaceutically acceptable salt and/or solvate thereof, or a composition comprising an effective amount of a compound of Formula (I), Formula (II) or Formula (III), or a pharmaceutically acceptable salt and/or solvate thereof, to a subject in need thereof.

The present application also includes use of a compound of Formula (I), Formula (II) or Formula (III), or a pharmaceutically acceptable salt and/or solvate thereof, or a composition comprising an effective amount of a compound of Formula (I), Formula (II) or Formula (III), or a pharmaceutically acceptable salt and/or solvate thereof, for forming ternary protein complexes, either in a biological sample or in a subject.

Further included is use of a compound of Formula (I), Formula (II) or Formula (III), or a pharmaceutically acceptable salt and/or solvate thereof, or a composition comprising an effective amount of a compound of Formula (I), Formula (II) or Formula (III), or a pharmaceutically acceptable salt and/or solvate thereof, for forming ternary protein complexes, either in a biological sample or in a subject.

The present application further includes use of a compound of Formula (I), Formula (II) or Formula (III), or a pharmaceutically acceptable salt and/or solvate thereof, or a composition comprising an effective amount of a compound of Formula (I), Formula (II) or Formula (III), or a pharmaceutically acceptable salt and/or solvate thereof, for recruiting an antibody or an immune cell for immunotherapy, either in a biological sample or in a subject.

The present application includes use of a compound of Formula (I), Formula (II) or Formula (III), or a pharmaceutically acceptable salt and/or solvate thereof, or a composition comprising an effective amount of a compound of Formula (I), Formula (II) or Formula (III), or a pharmaceutically acceptable salt and/or solvate thereof, for recruiting an antibody or an immune cell and targeting a cell for provoking an immune response to the cell, either in a biological sample or in a subject.

Also included is use of a compound of Formula (I), Formula (II) or Formula (III), or a pharmaceutically acceptable salt and/or solvate thereof, or a composition comprising an effective amount of a compound of Formula (I), Formula (II) or Formula (III), or a pharmaceutically acceptable salt and/or solvate thereof, for binding tumor antigens on a cell, either in a biological sample or in a subject.

Further included is use of a compound of Formula (I), Formula (II) or Formula (III), or a pharmaceutically acceptable salt and/or solvate thereof, or a composition comprising an effective amount of a compound of Formula (I), Formula (II) or Formula (III), or a pharmaceutically acceptable salt and/or solvate thereof, for treating a disease, disorder, or condition that is treatable by engaging an immune response.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DRAWINGS

Certain embodiments of the application will now be described in greater detail with reference to the attached drawings in which:

FIG. 1 shows schematic depictions of a universal synthetic antigen receptor (SAR) or antibody (Ab) used in conjunction with reversible PIMs (rPIMs), according to exemplary embodiments of the application.

FIG. 2 shows a schematic of the reversible reaction mechanism for imine formation in the DNP binding pocket according to exemplary embodiments of the application.

FIG. 3 shows chemical structures of rPIM components according to exemplary embodiments of the application.

FIG. 4A and FIG. 4B show SDS-PAGE gel images and schematic depictions of rPIMs where FIG. 4A shows engagement and FIG. 4B shows dissociation of anti-DNP monoclonal antibody (mAb) with a covalent imine linkage, each according to exemplary embodiments of the application.

FIG. 5A, FIG. 5B and FIG. 5C show graphs of biolayer interferometry (BLI) analysis and schematic depictions of the rPIMs reaction kinetics for FIG. 5A: association; FIG. 5B: dissociation; and FIG. 5C: combined association and dissociation to anti-DNP mAb, each according to exemplary embodiments of the application.

FIG. 6A and FIG. 6B show graphs of biolayer interferometry (BLI) analysis and schematic depictions of rPIM-induced ternary complex, where FIG. 6A shows association and FIG. 6B shows dissociation between uPAR and anti-DNP mAb, each according to exemplary embodiments of the application.

FIG. 7 shows a graph of AlphaLISA analysis and a schematic depiction of rPIM-induced bead-bead interactions according to exemplary embodiments of the application.

FIG. 8A and FIG. 8B show schematic depictions of exemplary modified primary T-cells where FIG. 8A shows DAP12-based synthetic antigen receptor (anti-DMP KIR-CAR) and FIG. 8B shows anti-DNP CD28/4-1BB expressed on primary T cells, each according to exemplary embodiments of the application.

FIG. 9A and FIG. 9B show T cell labeling using FIG. 9A: rPIMs and FIG. 9B a non-reversible PIM using flow cytometry, each according to exemplary embodiments of the application.

FIG. 10A, FIG. 10B, FIG. 10C and FIG. 10D show graphs representing the effects of rPIM reversible covalency on FIG. 10A: T cell activation; FIG. 10B: cytotoxicity against cancer cells; FIG. 10C: T cell proliferation; and FIG. 10D: antibody dependent phagocytosis (ADCP), each according to exemplary embodiments of the application.

FIG. 11 shows a graph of cytotoxicity against K562 tumor cells using rPIMs with different electrophiles according to exemplary embodiments of the application.

FIG. 12 shows graphs of cytotoxicity using rPIMs with T cells engineered with second-generation CARs—anti-DNP CD28-CAR T cells (left) and anti-DNP 4-1BB-CAR T cells (right)—according to exemplary embodiments of the application.

FIG. 13A, FIG. 13B and FIG. 13C show schematic depictions and graphs of cytotoxicity assays assessing reversibility of PIMs with FIG. 13A: media alone; FIG. 13B: DNP—KIR-CAR T cells alone in media; and FIG. 13C: DNP KIR-CAR T cells and A172 tumor cells, each according to exemplary embodiments of the application.

FIG. 14 shows the structure of glutamate urea lysine (GUL) targeting ligand which is clicked to DNP-electrophile scaffolds and used to target prostate specific membrane antigen (PSMA), each according to exemplary embodiments of the application.

FIG. 15 shows cytotoxicity of anti-DNP KIR-CAR T cells against PSMA expressing LnCAP nuclight red cells with PSMA-targeting rPIMs displaying enhanced cytotoxic function with CBD3 adapter compared to both irreversible covalent SuFEX and non-covalent Ph, each according to exemplary embodiments of the application.

FIG. 16 displays increased bioavailability of the CBD3 rPIM in solution at 24, 72, and 120 hours when cultured with T cells and tumor cells when compared to the irreversible covalent SuFEX PIM, according to exemplary embodiments of the application.

FIG. 17 shows the structure of integrin targeting “Knottin” Ligand containing the RGD domain for binding. This peptide is clicked to DNP warheads to generate our rPIMs, PIM, and NPIM, according to exemplary embodiments of the application.

FIG. 18 shows cytotoxicity of anti-DNP KIR-CAR T cells against U87-MG eGFP tumor cells which overexpress the av33 integrin. Co-culturing with varying concentrations of knottin CBD3 rPIM, knottin SuFEX PIM, or knottin Ph NPIM (0.5 nM-100 nM), displays that only the adapter containing the reversible CBD3 electrophile is able to facilitate cytotoxic function, each according to exemplary embodiments of the application.

FIG. 19 shows in vivo assessment of activity of integrin-targeting CBD3 rPIM and Ph NPIM (also referred to as rCIR and Ph NCIR) against a cell-derived U87-MG xenograft model, showing growth out till 15 days, according to exemplary embodiments of the application.

FIG. 20A and FIG. 20B show results of a fluorescence polarization assay and AlphaLISA binary complex assay; FIG. 20A: results demonstrating that rPIMs have different, electrophile-dependent dissociation rates and quantifies these rates by monitoring dissociation in real-time with fluorescence polarization, and FIG. 20B: AlphaLISA competition assay determines binary rPIM-mAb affinity by measuring the reversible, thermodynamic binding affinity of the rPIM-mAb interaction. Error bars=SEM, n=3 replicate measurements, each according to exemplary embodiments of the application.

FIG. 21A, FIG. 21B and FIG. 21C show reaction coordinate diagrams and relative energies of ligand binding followed by electrophile binding. The initial ligand binding step is equivalent across the rPIM series. FIG. 21A: The second, reversible binding step differs depending on the electrophile and dictates the overall affinity of the rPIM-protein interaction; FIG. 21B: reaction coordinate diagram of reversible covalent binding and competitor-induced dissociation. Reversible covalent engagement results after initial non-covalent ligand-protein association. The addition of competitor replaces this interaction, leaving the dissociation barrier measured reflective of imine hydrolysis alone; and FIG. 21C: reaction coordinate diagram highlighting barriers and equilibrium constants involved in reversible electrophile-protein binding, each according to exemplary embodiments of the application.

FIG. 22A, FIG. 22B and FIG. 22C show dual reversible covalent strategy for cell-cell proximity induction; FIG. 22A: a schematic showing how dual reversible covalent glue mimics bridge immune cells and tumor cells; FIG. 22B: X-ray crystal structure of uPAR bound to AE147. Two positions on AE147 were selected for electrophile placement. “Internal” position, on left, is 11.2 Å from the closest lysine residue in uPAR. “C-terminal” position, on right, is 9.2 Å from closest lysine residue in uPAR. Crystal structure sourced from PDB: 1YWH; and FIG. 22C: structures of reversible covalent glue mimics (rCGM) and control molecules. rCGM-1 (compound I-11) features dual 2-APBA electrophiles, covalently targeting both anti-DNP antibodies and uPAR simultaneously. rCGM-2 (compound 1-12) is covalent towards anti-DNP, but lacks a 2-APBA warhead on the uPAR-binding side. rCGM-3 (compound I-12) is covalent towards uPAR, but lacks covalency towards anti-DNP. rCGM-4 is a fully non-covalent control. rCGM-5 features dual aryl sulfonyl fluoride electrophiles, irreversibly targeting both anti-DNP and uPAR, each according to exemplary embodiments of the application.

FIG. 23A and FIG. 23B show AlphaLISA analysis of rCGM-induced ternary complex formation; FIG. 23A shows a schematic of the structure of the complex, and FIG. 23B: rCGM was incubated overnight with 5 nM of uPAR-biotin and 5 nM of SPE7, then combined with 10 μg/mL each of streptavidin donor and anti-IgG acceptor beads. Error bars=SEM, n=3 replicate measurements, each according to exemplary embodiments of the application.

FIG. 24A and FIG. 24B show rCGM-induced synthetic antigen receptor T cell killing of uPAR-expressing A172 targets; FIG. 24A shows a schematic of the interactions, and FIG. 24B cytotoxicity was assessed with live-cell imaging, each according to exemplary embodiments of the application.

FIG. 25A and FIG. 25B show schematic of antibody dependent cellular phagocytosis induced via dual reversible CGM bridging target cells and effectors cells;

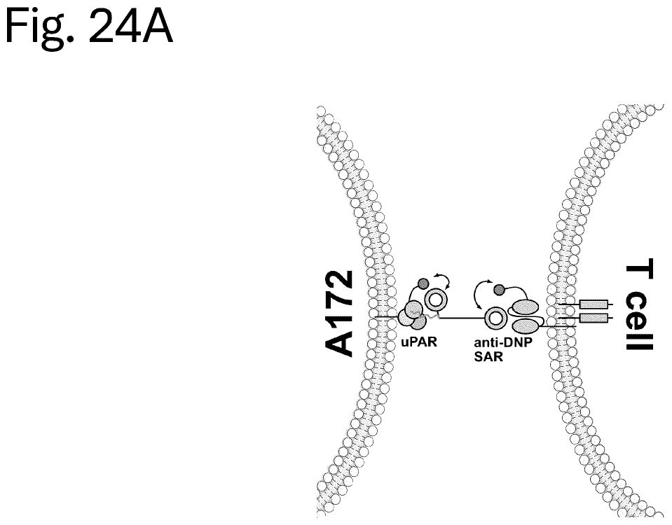

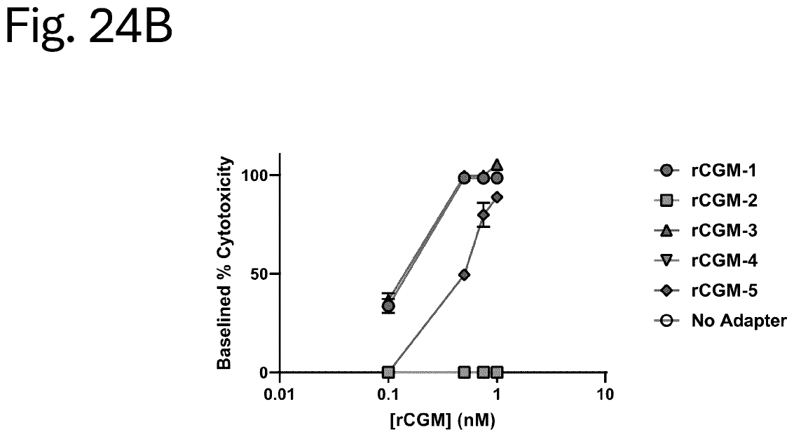

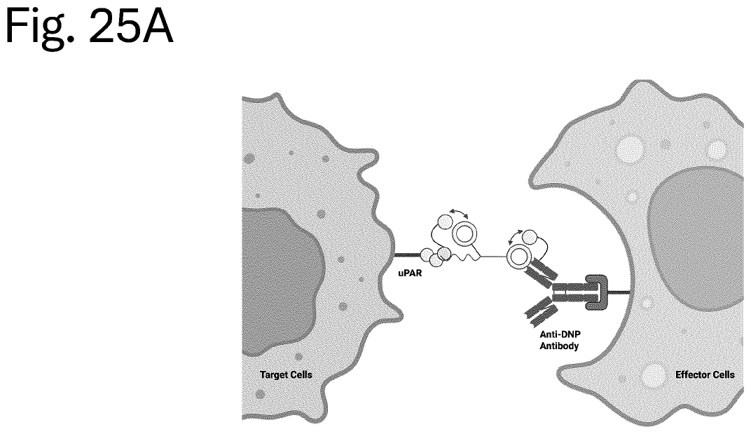

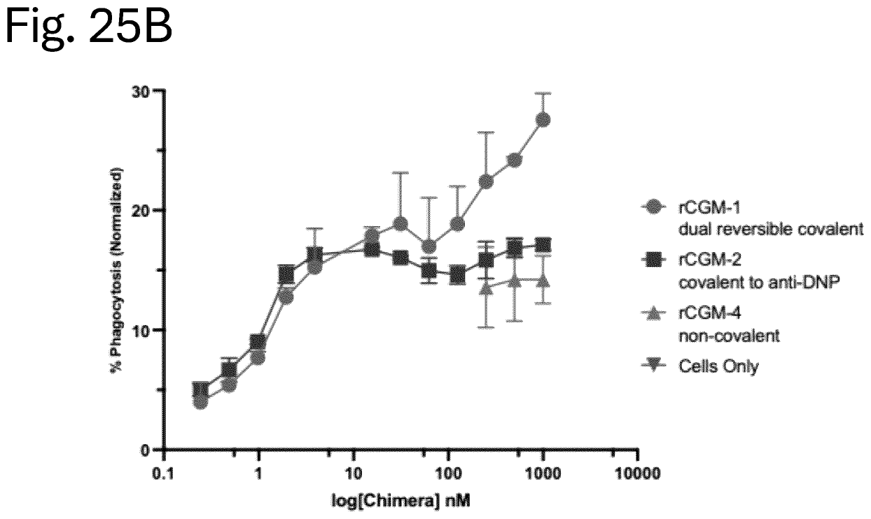

FIG. 24A shows a schematic of the interactions, and FIG. 25B shows two-colour flow cytometry experiment using ES-2 target cells and THP-1 monocytes. Various concentrations of rCGM-1 (compound I-11), rCGM-2 (compound 1-12), and rCGM-4 were incubated with ES-2 target cells for 30 min prior to addition of THP-1 monocytes for 1 hr, each according to exemplary embodiment of the application.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art. It is also to be understood that the terminology used herein is for the purpose of describing particular aspects only and is not intended to be limiting.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies. In addition, all ranges given herein include the end of the ranges and also any intermediate range points, whether explicitly stated or not.

As used in this application, the singular forms “a, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

The term “compound(s) of the application” or “compound(s) of the present application” and the like as used herein refers to a compound of Formula (I), (II) or (III) or pharmaceutically acceptable salts and/or solvates thereof.

The term “composition(s) of the application” or “composition(s) of the present application” and the like as used herein refers to a composition, such a pharmaceutical composition, comprising one or more compounds of the application.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₁₀alkyl means an alkyl group having 1, 2, 3, 4, 5, 6, 7, 8, 9 or 10 carbon atoms.

The term “alkylene”, whether it is used alone or as part of another group, means a bivalent straight or branched chain, saturated alkylene group, that is, a saturated carbon chain that contains substituents on two of its ends. The number of carbon atoms that are possible in the referenced alkylene group are indicated by the prefix “Cₙ₁₋₂”. For example, the term C₂₋₆alkylene means an alkylene group having 2, 3, 4, 5 or 6 carbon atoms.

The term “aryl” as used herein, whether it is used alone or as part of another group, refers to carbocyclic groups containing at least one aromatic ring and contains 6 to 20 carbon atoms.

The term “amine” or “amino,” as used herein, whether it is used alone or as part of another group, refers to groups of the general formula NR′R″, wherein R′ and R″ are each independently selected from hydrogen or C₁₋₆alkyl.

The term “amino acid” as used herein refers to an organic compound comprising amine (—NH₂) and carboxylic acid (—COOH) functional groups, along with a side-chain specific to each amino acid. The common elements of an amino acid are carbon, hydrogen, oxygen and nitrogen, though other elements are found in the side-chains of certain amino acids, including S and Se. Unless otherwise specified, an amino acid referenced herein is one of the 23 proteinogenic amino acids, that is amino acids that are precursors to proteins, and are incorporated into proteins during translation.

The following symbol:

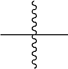

is used in chemical structures herein to represent a point of covalent attachment of a group to another group.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J.F.W. Ed., Plenum Press, 1973, in Greene, T. W. and Wuts, P.G.M., “Protective Groups in Organic Synthesis”, John Wiley & Sons, 3^{rd} Edition, 1999 and in Kocienski, P. Protecting Groups, 3^{rd} Edition, 2003, Georg Thieme Verlag (The Americas).

The term “linker” or “linker group” as used herein refers to any molecular structure that joins two or more other molecular structures together and that is compatible with a biological environment.

The term “compatible with a biological environment” as used herein it is meant that the chemical group or molecule is stable in, and/or does not denature, other molecules present in biological systems.

The term “biological systems” as used herein means any of a wide variety of systems which comprise proteins, enzymes, organic compounds, inorganic compounds, other sensitive biopolymers including DNA and RNA, and includes complex systems such as whole or fragments of plant, animal and microbial cells.

The term “subject” as used herein includes all members of the animal kingdom including mammals, and suitably refers to humans. Thus the methods and uses of the present application are applicable to both human therapy and veterinary applications.

The term “pharmaceutically acceptable” means compatible with the treatment of subjects.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with, the treatment of subjects.

The term “treats”, “treating” or “treatment” as used herein and as is well understood in the art, means an approach for obtaining beneficial or desired results, including clinical results. Beneficial or desired clinical results include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with early cancer can be treated to prevent progression, or alternatively a subject in remission can be treated with a compound or composition of the application to prevent recurrence. Treatment methods comprise administering to a subject a therapeutically effective amount of one or more of the compounds of the application and optionally consist of a single administration, or alternatively comprise a series of administrations.

“Palliating” a disease, disorder or condition means that the extent and/or undesirable clinical manifestations of a disease, disorder or condition are lessened and/or time course of the progression is slowed or lengthened, as compared to not treating the disorder.

The term “prevention” or “prophylaxis”, or synonym thereto, as used herein refers to a reduction in the risk or probability of a subject becoming afflicted with a disease, disorder or condition or manifesting a symptom associated with a disease, disorder or condition.

The term “immunotherapy” as used herein refers to the treatment of disease, disorder or condition by activating the immune system to produce or provoke an immune response.

The term “immune response” as used herein refers to the activation of immune cells.

The term “hapten” as used herein refers to a small molecule that can elicit an immune response only when attached to a large carrier such as a protein. The carrier may be one that also does not elicit an immune response by itself.

As used herein, the term “effective amount” or “therapeutically effective amount” means an amount of one or more compounds of the application that is effective, at dosages and for periods of time necessary to achieve the desired result. For example, in the context of treating a disease, disorder or condition, an effective amount is an amount that, for example, treats the disease, disorder or condition compared to without administration of the one or more compounds.

The term “administered” as used herein means administration of a therapeutically effective amount of one or more compounds or compositions of the application to a cell, tissue, organ or subject.

The term “neoplastic disorder” as used herein refers to a disease, disorder or condition characterized by cells that have the capacity for autonomous growth or replication, e.g., an abnormal state or condition characterized by proliferative cell growth. The term “neoplasm” as used herein refers to a mass of tissue resulting from the abnormal growth and/or division of cells in a subject having a neoplastic disorder.

The term “cancer” as used herein refers to cellular-proliferative disease states.

The term “reversible” as used herein refers to a covalent bond between two moieties being susceptible to hydrolysis, allowing both forward (association) and backward (dissociation) reaction pathways to take place.

The term “selective” as used herein having regard to chemical entities means that an entity is more likely to bind to a target site with little or no detectable binding to non-target sites over a particular time period.

It will be understood that any component defined herein as being included may be explicitly excluded by way of proviso or negative limitation, such as any specific compounds or method steps, whether implicitly or explicitly defined herein.

### II. Compounds and Compositions of the Application

Reversible covalent electrophiles in the context of PIMs that act as covalent reprogramming adapters, may allow for covalent anchoring in a similar manner to traditional PIMs, but also benefit from reversibility which, in principle, should enable the rPIM to release from a given ligand and bind to another.

Disclosed herein are “reversible” proximity inducing molecules (PIMs) that employ reversible covalent electrophiles to render the PIM impervious to inactivation by hydrolysis—which is part of the normal reversible reaction mechanism—and enable tunable engagement residence times, while facilitating cell turnover and “correction” of off-target labeling. Regarding the latter, labeling of the target protein(s) of interest is additionally thermodynamically stabilized which decreases the dissociation rate/reverse reaction associated with the reversible covalent electrophile. Without being bound to theory, when irreversible PIMs are hydrolyzed (e.g. sulfonyl fluoride->sulfonic acid), the hydrolyzed product can no longer engage nucleophilic amino acids in a covalent bond. However, rPIMs (such as 2-acetylphenylboronic acid electrophiles) are not inactivated by hydrolysis. rPIM alone cannot be hydrolyzed, but instead is in equilibrium with its hydrate (ketone/aldehyde+water<->geminal diol). Hydrolysis of the rPIM-protein adduct returns the protein and the rPIM intact as well (rPIM+protein <->water+rPIM-protein). In embodiments of the application, the hydrolysis conditions are under physiological conditions, for example at a temperature range of about 20° C. to about 40° C., atmospheric pressure and pH of about 6 to about 7.

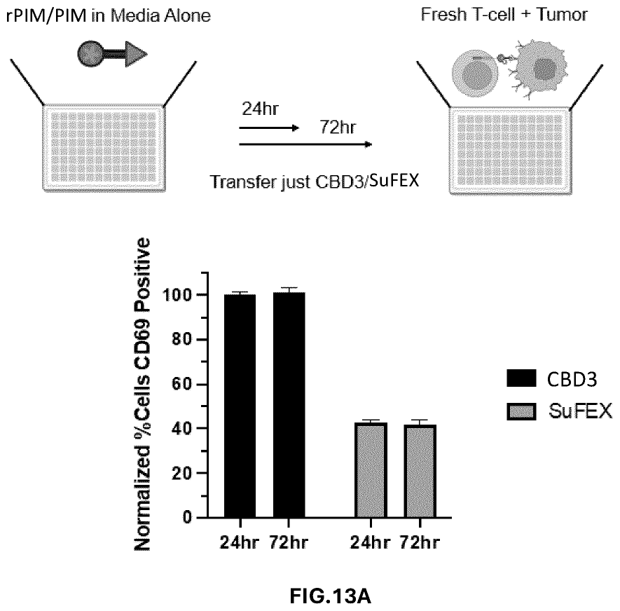

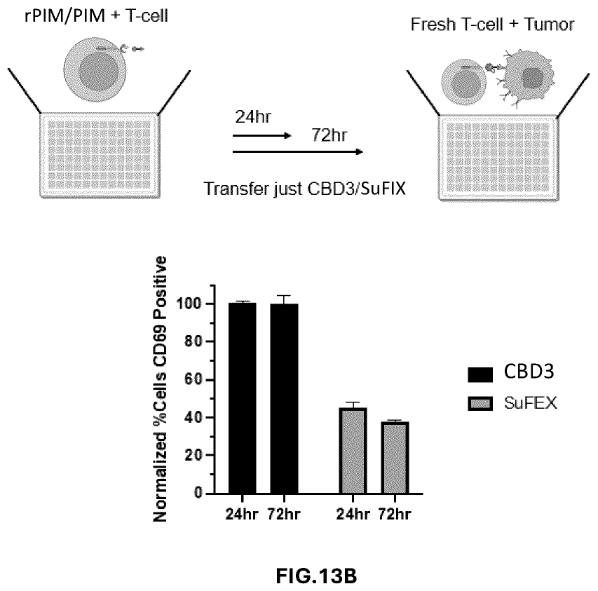

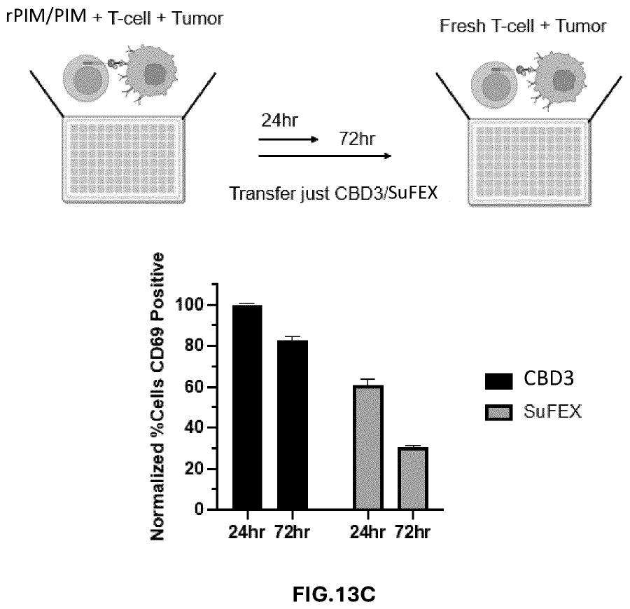

Without wishing to be bound to theory, reversibility reduces hydrolysis and extends the activity of the rPIM relative to conventional PIMs which inactivate upon hydrolysis (see for example FIG. 13). The reversible chemistry may also allow to place electrophiles on complex molecules containing nucleophiles that would otherwise self-inactivate with a non-reversible chemistry, thus extending the covalent technology beyond small molecule ligands.

Multiple configurations of the PIMs were designed and synthesized. Using a variety of in vitro systems, the enhanced biological performance of reversible covalent electrophiles has been validated in the context of PIMs to generate the reversible PIMs (rPIMs) of the present application. In some embodiments, rPIMs are designed with a dinitrophenyl (DNP) ligand to mediate proximity induction of the rPIM with antibodies or synthetic immune receptors specific for DNP; the synthetic immune receptors serving as a platform engineering, for example, immune cells that can be directed to attack tumors, and other targets, via rPIMs. These two systems validate the utility of rPIMs for tumor immunotherapies that operate via Fc receptor effector cell activation (e.g. antibody-dependent cell phagocytosis), and synthetic receptor-mediated cell activation (e.g. cytotoxicity by engineered CAR-T cells). In some embodiments, rPIMs comprise three domains: a dinitrophenyl (DNP) hapten that non-covalently binds to immune receptors, a reversible covalent electrophilic moiety associated with different reversible reaction kinetics, and a peptide disease target-binding domain. In some embodiments, rPIMs comprise four domains: a dinitrophenyl (DNP) hapten that non-covalently binds to immune receptors, two covalent reversible electrophile moieties associated with different reversible reaction kinetics, and a peptide disease target-binding domain.

In addition to being a class of synthetic covalent tumor immunotherapeutic, rPIMs serve as chemical tools to interrogate key questions surrounding immune receptor pharmacology including the role of target residence time, ternary complex kinetic stability, and mechanical strain at the immune synapse, in addition to the importance and timescales of receptor internalization and immune cell processive killing/turnover.

In some embodiments, an arylboronic acid electrophile-substituted rPIM significantly increases ternary complex formation and tumor immunotherapeutic function compared to an irreversible covalent SuFEX (such as sulfonyl fluoride or fluorosulfate)-substituted PIM highlighting the benefit of reversible covalency for proximity inducing molecules and bi-functionals. In some embodiments, the derivative termed “CBD3”, 2-acetylphenylboronic acid electrophile, has high thermodynamic affinity for the target antibody/receptor due to avidity but also the fast reversible reaction kinetics (i.e. fast nucleophilic attack and fast hydrolysis in the reverse reaction) suggesting it meets a prime balance between kinetically stabilizing ternary complexes between tumor and immune cells but also enables efficient processive killing and immune cell turnover.

rPIMs were developed as dual reversible covalent bi-functionals (also referred to herein as covalent glue mimics or CGMs), and show that dual rPIMs induce higher levels of ternary complex in comparison to all other modalities. Dual rPIMs also show functional enhancements in phagocytosis and T cell killing.

Herein it is demonstrated that the pre-organization of one or two electrophiles on a single bifunctional molecule can selectively and reversibly crosslink two non-interacting proteins, stabilizing the ternary complex. Collectively, this application describes reversible covalent proximity inducing molecules with therapeutic potential, and underscores their utility for driving biomolecular interactions that lack positive co-operativity.

Accordingly, provided is a compound of Formula (I), Formula (II) or Formula (III) or a pharmaceutically acceptable salt and/or solvate thereof,

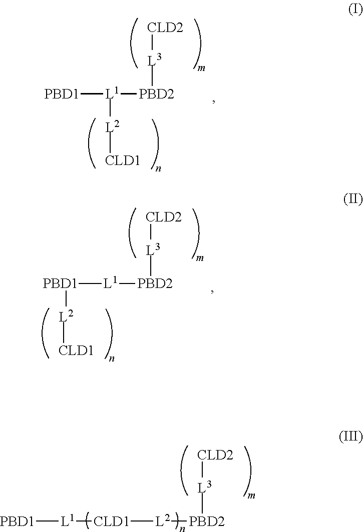

wherein PBD1 and PBD2 are each independently a protein binding domain (PBD) and CLD1 and CLD2 are each independently a covalent labeling domain (CLD), and L¹, L² and L³ are each independently a linker group;
n=0 or 1 and m=0 or 1, wherein at least one of n and m is 1,
wherein PBD1 selectively binds to a first target protein and PBD2 selectively binds to a second target protein, and wherein CLD1, if present, comprises a functional group that, upon binding of PBD1 to the first target protein, forms an reversible covalent bond with a nucleophilic group in the first target protein and CLD2, if present, comprises a functional group that, upon binding of PBD2 to the second target protein, forms an reversible covalent bond with a nucleophilic group in the second target protein.

### Protein Binding Domains (PBD)

Protein Binding Domains (PBDs) of the present application are chemical entities comprising moieties which bind to target proteins to bring the proteins in proximity, intended to reversibly covalently crosslink two proteins within ternary complexes, providing proximity inducing stabilization. In some embodiments, the PBDs of the application are each selected to bind a target protein so that induced proximity will generate a beneficial effect. For example, the induced cell-cell proximity in three distinct tumor immunotherapeutic model systems (Macrophage based, NK cell based, and T cell based), leading to significant functional enhancements are provided. In some embodiments, one of the PBDs binds to a target protein such that it will be retained in a particular site of a subject such as a biological structure for example an organ or tissue or a pathological structure for example a tumor, with little or no detectable accumulation and/or retention in non-target sites over a particular time period, while the other PBD binds to a protein that has a benefit when brought in proximity of the organ, tissue or pathological structure.

Known uses of proximity inducing molecules include bringing together two proteins to allow for protein degradation (for example, by hijacking the ubiquitin-proteasome system) for example to degrade known disease-causing or -driving proteins such as oncogenic proteins, to increase protein stabilization, to target cells for immunotherapy, among other known uses.

In some embodiments, one of the PBD binds to a target protein, for example a protein that is overexpressed in a disease, disorder or condition such as cancer, and the other PBD binds to a target protein that acts as an effector to effect a beneficial effect including, without limitation, protein degradation, protein stabilization, cytotoxicity, phagocytosis and apoptosis. Target proteins and their PBDs are known and the selection of suitable PBDs for a particular therapeutic use can be made by a person skilled in the art. PBDs include, but are not limited to, small molecules such as protein binding compounds, enzyme inhibitors or pharmaceutical-like compounds.

In some embodiments, a first PBD binds to a synthetic antigen receptor on the surface of an immune cell, resulting in a primed immune cell that, via the second PBD, targets a protein on a cell of interest. In some embodiments, synthetic antigen receptors comprise an acceptor moiety that is bound by the first PBD, and an effector domain which effects signaling in an engineered cell. Exemplary synthetic antigen receptors are described in WO2023/230729, incorporated herein by reference.

In some embodiments, PBD1 and PBD2, are each different and independently comprise a moiety that binds to antigens on the surface of a target cell. In some embodiments, one of PBD1 and PBD2 comprises a moiety that binds to antigens on the surface of a target cell and the other of PBD1 and PBD2 comprises a moiety that binds to an immune effector. In some embodiments, one of PBD1 and PBD2 comprises a moiety that binds to proteins on the surface of a target cell and the other of PBD1 and PBD2 comprises a moiety that binds to an antibody. In some embodiments, one of PBD1 and PBD2 is a glutamate urea ligand that binds to prostate specific membrane antigen (PSMA) and the other of PBD1 and PBD2 comprises a moiety that binds to an effector, such as an immune cell or an antibody. In some embodiments, one of PBD1 and PBD2 comprises any of the hapten groups described in U.S. Pat. No. 9,296,708. Accordingly, in some embodiments one of PBD1 and PBD2 is a domain that binds to an antibody and is selected from the following groups:
1) a di- or trinitrophenyl group having the following structure:

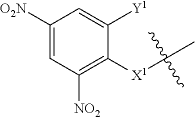

wherein Y¹ is H or NO₂;
X¹ is NR¹, O, CH₂, S(O), SO₂, SO₂O, OSO₂ or OSO₂O; and
R¹ is H, C14alkyl or C(O)C₁₋₄alkyl;
   2) a bicyclic nitro-substituted aromatic group having the following structure:

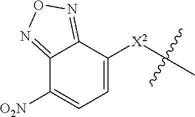

wherein X² is a bond, O, CH₂, NR² or S; and
R² is H, C₁₋₄alkyl or C(O)C₁₋₄alkyl;
   3) a galactose-containing carbohydrate having the following structure:

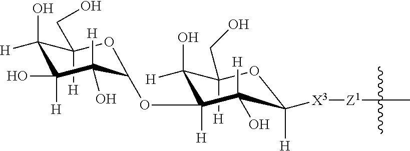

wherein X³ is CH₂, O, NR³ or S;
R³ is H or C₁₋₄alkyl; and
Z¹ is a bond, monosaccharide, disaccharide, oligosaccharide, glycoprotein or glycolipid; and
   4) a group having the following structure:

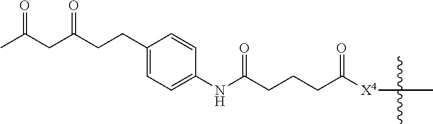

wherein X⁴ is O, CH₂ or NR⁴; and
R⁴ is H, C₁₋₄alkyl or C(O)C₁₋₄alkyl.

In some embodiments, X¹ is NR¹ and R¹ is H or C₁₋₃alkyl. In some embodiments Y¹ is H.

In some embodiments, X² is a bond or NR² and R² is H or C₁₋₃alkyl.

In some embodiments, X³ is O or NR³ and R³ is H or C₁₋₃alkyl. In some embodiment, Z¹ is a bond. In some embodiments, Z¹ is a monosaccharide or a disaccharide. In some embodiments, the monosaccharide is an aldose such as aldotriose (D-glyceraldehdye, among others), aldotetrose (D-erythrose and D-Threose, among others), aldopentose, (D-ribose, D-arabinose, D-xylose, D-lyxose, among others) or aldohexose (D-allose, D-altrose, D-Glucose, D-Mannose, D-gulose, D-idose, D-galactose and D-Talose, among others). In some embodiment, the monosaccharide is a ketose such as ketotriose (dihydroxyacetone, among others), ketotetrose (D-erythrulose, among others), ketopentose (D-ribulose and D-xylulose, among others) or ketohexose (D-Psicone, D-Fructose, D-Sorbose, D-Tagatose, among others). In some embodiments the monosaccharide is an aminosugar such as galactoseamine, sialic acid, N-acetylglucosamine, among others or a sulfosugar such as sulfoquinovose, among others. In some embodiments Z is a disaccharide such as sucrose (which may have the glucose optionally N-acetylated), lactose (which may have the galactose and/or the glucose optionally N-acetylated), maltose (which may have one or both of the glucose residues optionally N-acetylated), trehalose (which may have one or both of the glucose residues optionally N-acetylated), cellobiose (which may have one or both of the glucose residues optionally N-acetylated), kojibiose (which may have one or both of the glucose residues optionally N-acetylated), nigerose (which may have one or both of the glucose residues optionally N-acetylated), isomaltose (which may have one or both of the glucose residues optionally N-acetylated), β,β-trehalose (which may have one or both of the glucose residues optionally N-acetylated), sophorose (which may have one or both of the glucose residues optionally N-acetylated), laminaribiose (which may have one or both of the glucose residues optionally N-acetylated), gentiobiose (which may have one or both of the glucose residues optionally N-acetylated), turanose (which may have the glucose residue optionally N-acetylated), maltulose (which may have the glucose residue optionally N-acetylated), palatinose (which may have the glucose residue optionally N-acetylated), gentiobiluose (which may have the glucose residue optionally N-acetylated), mannobiose, melibiose (which may have the glucose residue and/or the galactose residue optionally N-acetylated), melibiulose (which may have the galactose residue optionally N-acetylated), rutinose, (which may have the glucose residue optionally N-acetylated), rutinulose or xylobiose, among others. In some embodiments Z¹ is an oligosaccharide such as any sugar of three or more (up to about 100) individual sugar (saccharide) units as described above (i.e., any one or more saccharide units described above, in any order, especially including glucose and/or galactose units as set forth above), or for example, fructo-oligosaccharides, galactooligosaccharides or mannan-oligosaccharides ranging from three to about ten-fifteen sugar units in size. In some embodiments, Z¹ is a glycoprotein such as N-glycosylated or O-glycosylated glycoproteins, including the mucins, collagens, transferring, ceruloplasmin, major histocompatability complex proteins (MHC), enzymes, lectins, selectins, calnexin, calreticulin, or integrin glycoprotein IIb/IIa, among others. In some embodiments Z¹ is a glycolipid such as a glyceroglycolipid (galactolipids or sulfolipids) or a glycosphingolipid, such as cerebrosides, galactocerebrosides, glucocerebrosides (including glucobicaranateoets), gangliosides, globosides, sulfatides, glycophosphphingolipids or glycocalyx, among others.

In some embodiments, Z¹ is a bond or a glucose or glucosamine (such as N-acetylglucosamine). In some embodiments, Z¹ is linked to a galactose residue through a hydroxyl group or an amine group on the galactose of Gal-Gal, suitably a hydroxyl group.

In some embodiments, X⁴ is NR⁴ and R⁴ is H or C₁₋₃alkyl.

In some embodiments, one of PBD1 and PBD2 is:

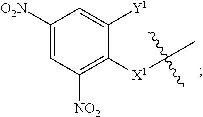

wherein
Y¹ is H or NO₂, suitably H; and
X¹ is NH or O, suitably NH.

In some embodiments, PBD1 and PBD2, are each different and independently comprise a moiety that binds to antigens on the surface of a target cell. In some embodiments, one of PBD1 and PBD2 comprises a moiety that binds to antigens on the surface of a target cell and the other of PBD1 and PBD2 comprises a moiety that binds to an immune cell In some embodiments, one of PBD1 and PBD2 comprises a moiety that binds to proteins on the surface of a target cell and the other of PBD1 and PBD2 comprises a moiety that binds to an antibody. In some embodiments, one of PBD1 and PBD2 is a glutamate urea ligand that binds to prostate specific membrane antigen (PSMA), such as glutamate urea lysine, and the other of PBD1 and PBD2 comprises a moiety that binds to an immune effector, such as an immune cell or an antibody. In some embodiments, one of PBD1 and PBD2 is selected from any of the PBD groups described in U.S. Pat. No. 9,296,708 for targeting a cell, incorporated herein by reference. Accordingly, in some embodiments, one of PBD1 and PBD2 is a domain that targets a cell and is selected from the following groups:

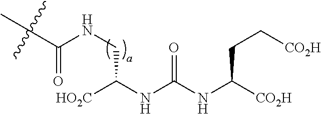

wherein a is an integer from 0 to 15, 1 to 15, 1 to 10, 1 to 8, or 1, 2, 3, 4, 5 or 6;

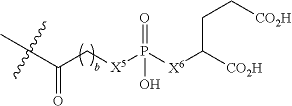

wherein X⁵ and X⁶ are independently CH₂, O, NH or S; and
b is an integer from 0 to 15, 1 to 15, 1 to 10, 1 to 8, or 1, 2, 3, 4,5 or 6;

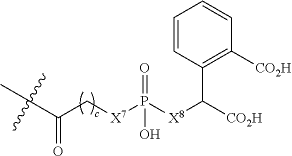

wherein X⁷ and X⁸ are independently CH₂, O, NH or S; and
c is an integer from 0 to 15, 1 to 15, 1 to 10, 1 to 8, or 1, 2, 3, 4, 5 or 6;

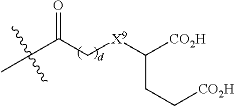

wherein X⁹ is O, CH₂, NR⁵, S(O), SO₂, SO₂O, OSO₂ or OSO₂O;
R⁵ is H, C₁₋₄alkyl or C(O)C₁₋₄alkyl; and
d is an integer from 0 to 15, 1 to 15, 1 to 10, 1 to 8, or 1, 2, 3, 4, 5 or 6; and
biotin or a biotin analog such as:

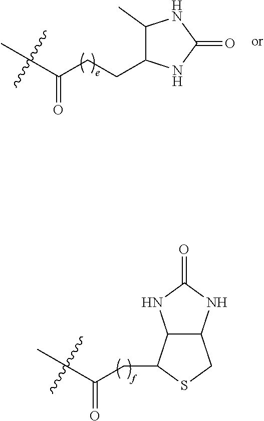

wherein e and f are, independently, an integer from 0 to 15, 1 to 15, 1 to 10, 1 to 8, or 1, 2, 3, 4, 5 or 6.

In some embodiments, a, b, c, d, e and f are independently 1, 2, 3, 4, 5 or 6, suitably 2, 3 or 4, more suitably 4.

In some embodiments, one of PBD1 and PBD2 comprises other tumor antigen binding ligands such as synthetic peptides against uPAR or HER2, folate receptor binding molecules such as folate or methotrexate, TLR agonists, or PD-1/PD-L1 antagonists.

In some embodiments, one of PBD1 and PBD2 comprises a hapten which binds to an antibody that is endogenous in a subject. In some embodiments, the antibody is present in the subject prior to treatment (i.e. the antibody levels do not have to be raised in the subject prior to treatment). In some embodiments, the antibody that is endogenous in the subject is an anti-dinitrophenyl (DNP) IgG. In some embodiments, the anti-DNP IgG is present in the subject's serum. In some embodiments, one of PBD1 and PBD2 is a hapten comprising a DNP for binding the anti-DNP IgG.

In some embodiments, the hapten comprises an electron deficient aryl or a carbohydrate. In some embodiments, the electron deficient aryl group is di- or trinitro phenyl. In some embodiments, the carbohydrate comprises digalactose.

In some embodiments, the PBD1 and PBD2 are the same, for example, where dimerization or blockade of dimerization of the same protein is desired.

In some embodiments, one of PBD1 and PBD2 is independently selected from a variety of monosaccharides or multivalent derivatives thereof recognized by several different serum carbohydrate specific antibodies such as anti-rhamnose and N-acetylglucosamine. In some embodiments, one of PBD1 and PBD2 independently comprises synthetic ligands such as cyclic peptides that bind all serum IgG.

In some embodiments, one of PBD1 and PBD2 comprises Fc receptor (FcR) targeting domains (FTDs), which bind a target protein being a cognate FcR, and the corresponding covalent labeling domain (CLD) comprises a functional group that, on binding of the FTD to the cognate FcR, forms a reversible covalent bond with a nucleophilic group in the FcR. In such embodiments, the other of PBD1 and PBD2 comprises a moiety that binds a protein that is the intended target of the immune cell.

In some embodiments, the FcR targeting domain (FTD) is a synthetic molecule comprising a binding domain which binds to a cognate FcR, on an immune cell. Any suitable FTD may be used depending on the specific FcR being targeted. In some embodiments, the FTD binds FcγR, optionally selected from CD64, CD32, CD16a, and CD16b.

In some embodiments, the FcR is CD64. In some embodiments, the FTD comprises circular peptide 33 (CP33) having the sequence VNSCLLLPNLLGCGDD (SEQ ID NO: 1), wherein C4 and C13 form a disulfide bond, or a functional variant thereof. In some embodiments, the FTD comprises a circular peptide 33 (CP33) having the sequence VNSCLLLPNLLGCDGD (SEQ ID NO: 2), wherein C4 and C13 form a disulfide bond, or a functional variant thereof. In some embodiments, the FTD comprises a CP33 having the sequence of SEQ ID NO: 3, SEQ ID NO: 4, SEQ ID NO: 7, SEQ ID NO: 8, SEQ ID NO: 9, SEQ ID NO: 10, SEQ ID NO: 11, or SEQ ID NO: 12, or functional variants thereof. In some embodiments, CP33 comprises a peptide having the sequence of SEQ ID NO: 3, wherein X is K and wherein C5 and C14 form a disulfide bond. In some embodiments, CP33 comprises a peptide having the sequence of SEQ ID NO: 4, wherein X is K and wherein C5 and C14 form a disulfide bond. In some embodiments, CP33 comprises a peptide having the sequence of SEQ ID NO: 7, wherein X is K and wherein C7 and C16 form a disulfide bond. In some embodiments, CP33 comprises a peptide having the sequence of SEQ ID NO: 8, wherein X is K and wherein C7 and C16 form a disulfide bond. In some embodiments, CP33 comprises a peptide having the sequence of SEQ ID NO: 9, wherein X is K and wherein C5 and C14 form a disulfide bond. In some embodiments, CP33 comprises a peptide having the sequence of SEQ ID NO: 10, wherein X is K and wherein C5 and C14 form a disulfide bond. In some embodiments, CP33 comprises a peptide having the sequence of SEQ ID NO: 11, wherein X1 is K, wherein X18 is K, and wherein C5 and C14 form a disulfide bond. In some embodiments, CP33 comprises a peptide having the sequence of SEQ ID NO: 12, wherein X1 is K, wherein X18 is K, and wherein C5 and C14 form a disulfide bond. In some embodiments, the CP33 peptide is C-terminally amidated. In some embodiments, the CP33 peptide is not C-terminally amidated.

In some embodiments, the PBD comprises other tumor antigen binding ligands such as, without limitation, synthetic peptides which bind uPAR (e.g. as described in Ploug M, et al., Peptide-derived antagonists of the urokinase receptor, affinity maturation by combinatorial chemistry, identification of functional epitopes, and inhibitory effect on cancer cell intravasation. Biochemistry. 2001 Oct. 9; 40(40):12157-68, incorporated herein by reference, or functional variants thereof). In an embodiment, the synthetic peptide which binds uPAR comprises an amino acid sequence of L-Lys-Gly-Gly-L-Ser-Gly-L-Asp-L-Cha-L-Phe-D-Ser-D-Arg-L-Tyr-L-Leu-L-Trp-L-Ser (SEQ ID NO: 13), or a functional variant thereof. In an embodiment, the synthetic peptide which binds uPAR comprises an amino acid sequence of L-Lys-Gly-Gly-L-Ser-Gly-L-Asp-L-Cha-L-Phe-D-Ser-D-Arg-L-Ala-L-Leu-L-Trp-L-Ser (SEQ ID NO: 14) as used herein, or functional variant thereof. In some embodiments, the PBD comprises other tumor antigen binding ligands such as, without limitation, HER2, folate receptor binding molecules such as folate or methotrexate, Toll-like receptor (TLR) agonists, or PD-1/PD-L1 antagonists. The PBD may also include Integrin binding ligands such as the Knottin peptide and other RDG mimetics. The PBD may also include therapeutic antibodies, scFv, aptamers, and other biologics.

In some embodiments, one of PBD1 and PBD2 comprises knottin (inhibitor cystine knot) of formula:

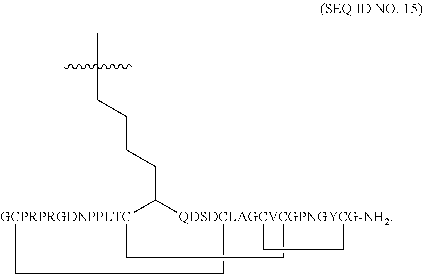

### Covalent Labeling Domains (CLD)

Covalent Labeling Domains (CLD), including CLD1 and CLD2, are labelling domains comprising a functional group that forms a reversible covalent bond with a nucleophile in the target protein that is proximal to the corresponding PBD binding site. By “proximal” it is meant that the nucleophile is located in an area that, when the compound of the application is bound to the target protein via one of the PBDs, the nucleophile is in a spatial location to react with the CLD. For example, in some embodiments, the distance between the nucleophile and the CLD is about 2 Å to about 10 Å. In some embodiments, CLD1 and CLD2 each independently comprise an electrophilic functional group that reacts with a nucleophilic moiety of an amino acid in the first and second target proteins, respectively. In some embodiments, the amino acid nucleophile is an amine (NH₂) or a thiol (SH), such as lysine or cysteine.

In some embodiments, CLD1 and CLD2 each independently comprise an arylboronic acid, a benzaldehyde, a nitrile, an acrylamide or a ketoamide functional group.

In some embodiments, CLD1 and CLD2 each independently comprise an aldehyde, a boronic acid, an amide, a nitrile and/or a ketone. In some embodiments, lysine-targeting electrophiles include salicylaldehydes, cysteine-targeting electrophiles include cyanoacrylamides, amide-substituted acrylamides, trifluoromethyl ketones, and nitriles, and serine and threonine-targeting electrophiles include boronic acids and alpha-ketoamides.

A person skilled in the art would appreciate that there are many other functional groups that may be used in CLD1 and/or CLD2. Such group would be compatible with a biological environment and would react with a nucleophile to form a covalent bond.

In some embodiments, CLD1 and CLD2 each independently comprise:

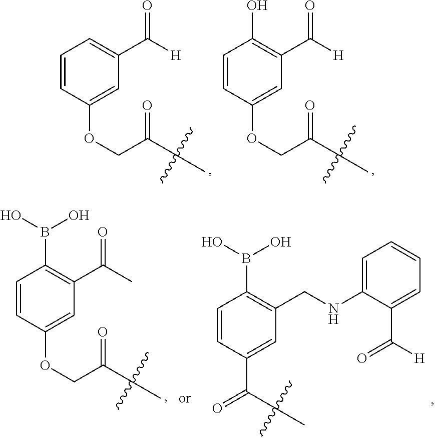 [SMILES: *C(=O)COc1cccc(C=O)c1 ; *C(=O)COc1ccc(O)c(C=O)c1 ; *C(=O)COc1ccc(B(O)O)c(C(C)=O)c1 ; *C(=O)c1ccc(B(O)O)c(CNc2ccccc2C=O)c1]

herein identified as CBD1 (benzaldehyde), CBD2 (2-hydroxybenzaldehyde), CBD3 (2-acetylphenylboronic acid), and CBD4 ((2-(((2-formylphenyl)amino)methyl)phenyl)boronic acid), respectively.

In some embodiments, CLD1 and CLD2 functional groups are advantageous as they undergo significant increases in proximal labeling-effective molarity, react with diverse amino acids proximal to the binding site, and can be efficiently incorporated into complex PIM molecular formats including peptides or carbohydrates.

Other functional groups may be used in CLD1 and/or CLD2. Such group would be compatible with a biological environment and would react with a nucleophile in the target protein to form a reversible covalent bond.

### Linker Groups

A person of skill in the art would appreciate that the linkers L¹, L² and L³ should have a length and spatial orientation appropriate to link the PBD moieties together and/or through a CLD, and the CLD moieties with the remainder of the molecule. In some embodiments, a linker is also incorporated to provide appropriate distance and/or spatial orientation of a protein binding domain and a covalent labeling domain towards a target protein. In some embodiments, the linker rigidity and length is tuned to maximize labeling kinetics and further comprises rigidifying elements such as carbocycles, heterocycles, aromatics and/or heteroaromatics.

Linkers are any molecular structure that joins two or more other molecular structures together and that are compatible with a biological environment. In some embodiments, the linker moiety comprises at least one functional group selected from an ester, amide, ether, thioether, thioamide, thioester and amine.

In some embodiments, L¹, L² and L³, are independently a direct bond, C₁₋₂₀ alkylene, optionally interrupted by triazolyl, piperidinyl, pyrrolidinyl, tricyclo- or a triazolyl fused with a 9- to 16-membered heterocyclyl or carbocyclyl ring, and/or one or more heteromoieties such as O, S, S(O), SO₂, OSO₂, SO₂O, OSO₂O, NR⁸, C(O), NHC(O), or C(O)NH, wherein R⁸ is H or C₁₋₄alkyl. In some embodiments, L¹, L² and L³ each independently comprises a group having the following structure:

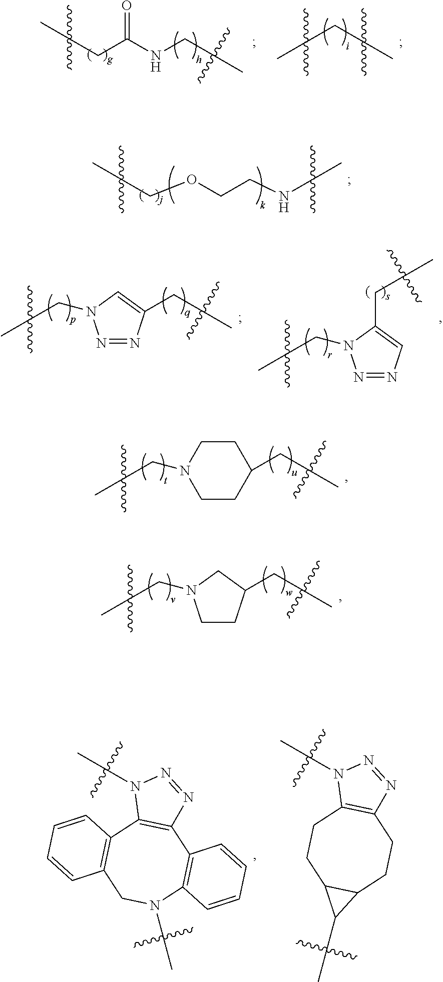

or any combination thereof,
wherein, g, h, i, j, k, p, q, r, s, t, u, v and w are, independently, 1, 2, 3, 4, 5, 6, 7, 8, 9 or 10.

In some embodiments, j is 2 and k is 3. In some embodiments, g is 1 and h is 2. In some embodiments, i is 1, 2, 3, 4, 5, 6, 7, 8, 9 or 10. In some embodiments, p, q, r and s are, independently, 1, 2, 3 or 4.

In some embodiments, L¹, L² and L³, are independently a direct bond,

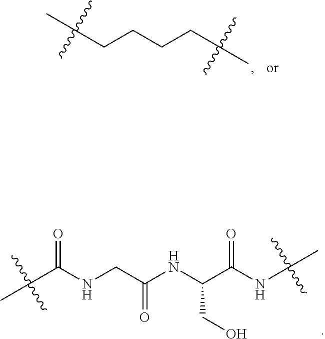 [SMILES: *CCCC* ; *NC(=O)[C@H](CO)NC(=O)CNC(*)=O]

In some embodiments, the present application includes a compound selected from Table 1.

TABLE 1
Compounds of the application

| Cpd | |
|---|---|
| No. | Structure |
| I-1 | 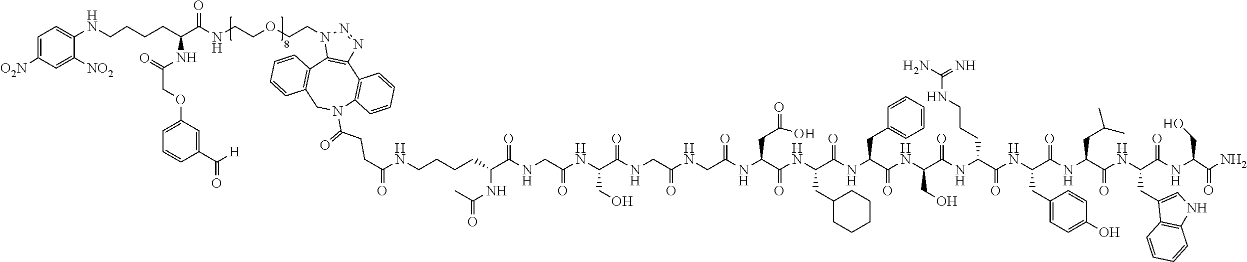 |
| | 2C |
| I-2 | 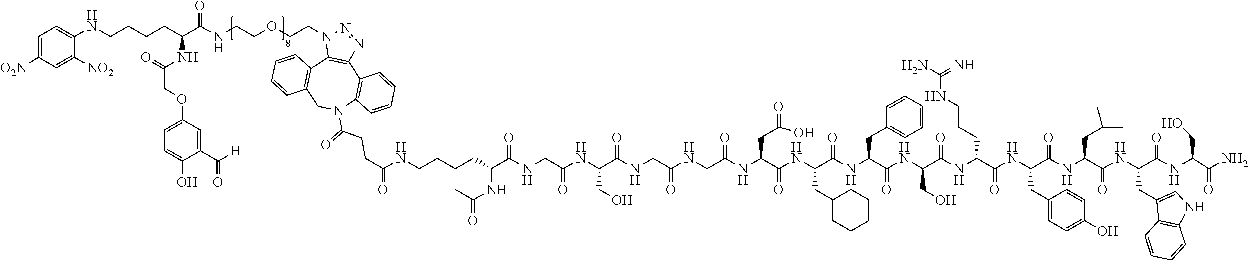 |
| | 3C |
| I-3 | 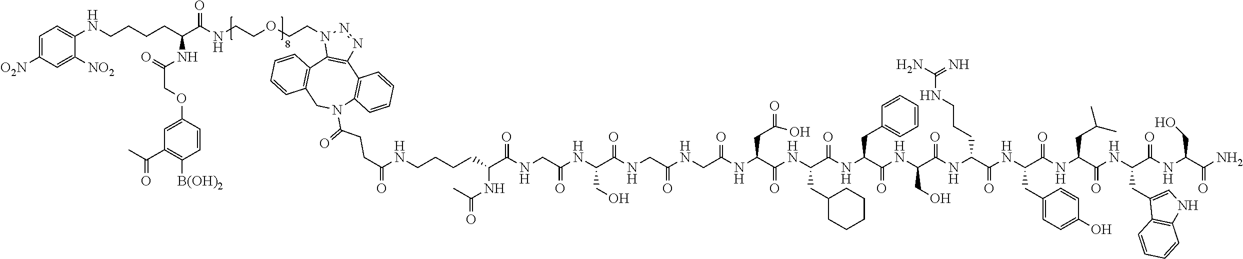 |
| | 4D |
| I-4 | 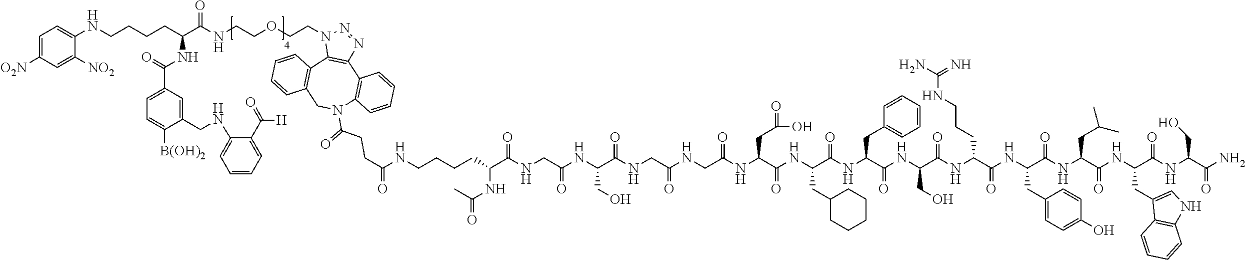 |
| | 5C |
| I-5 | 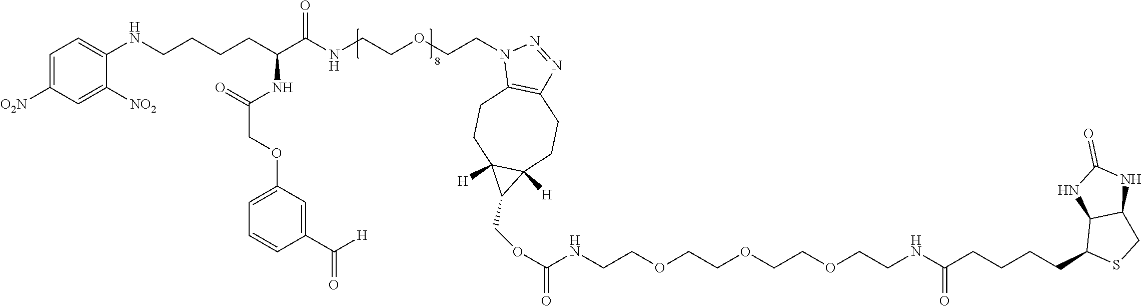 |
| | 2D |
| I-6 | 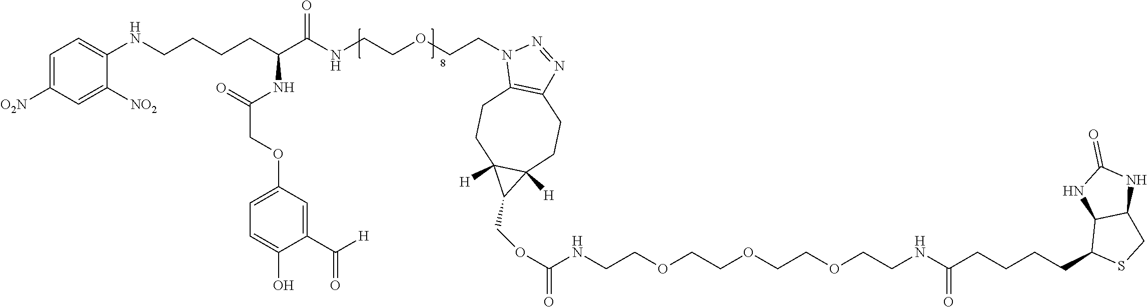 |
| | 3D |
| I-7 | 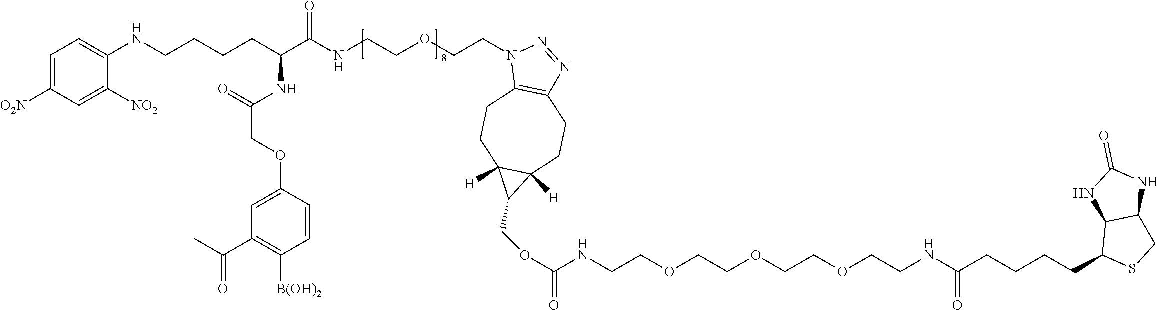 |
| | 4D |
| I-8 | 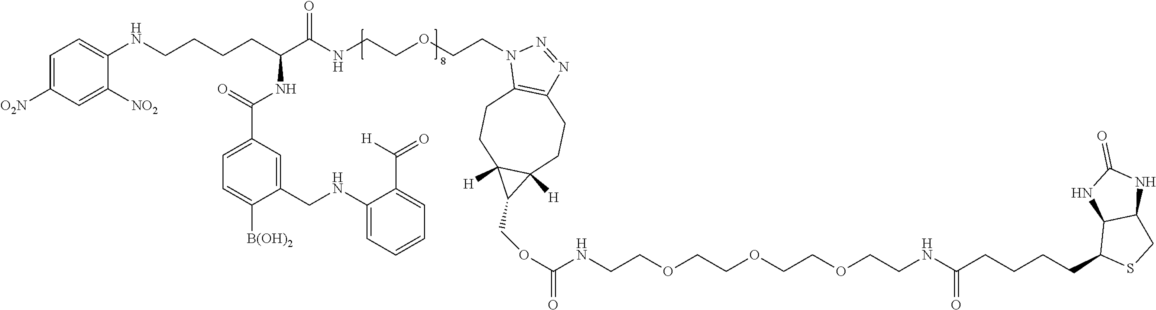 |
| | 5D |
| I-9 | 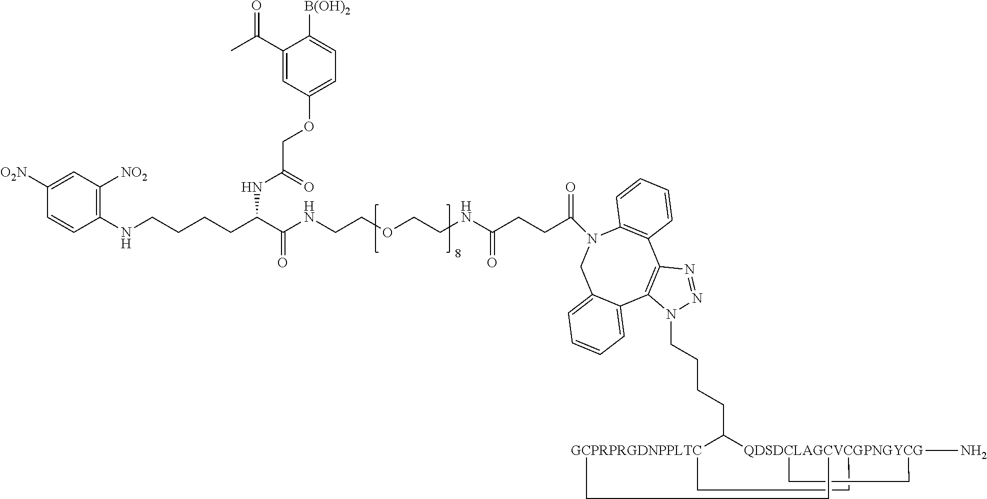 |
| | 4E |
| I-10 | 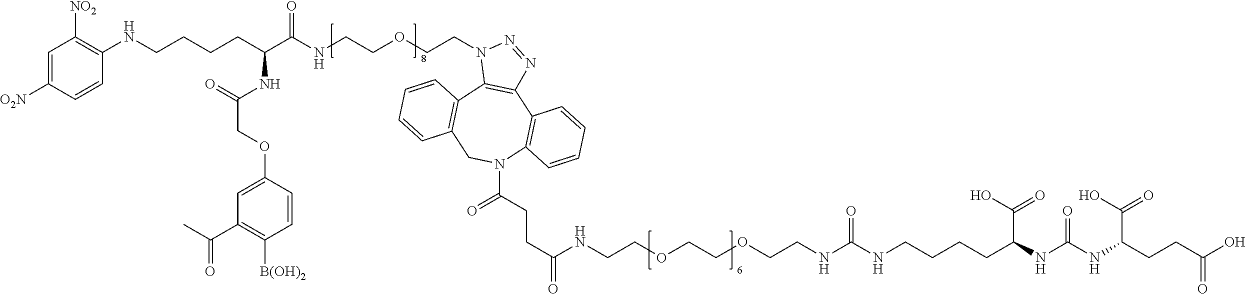 |
| | 4F |
| I-11 | 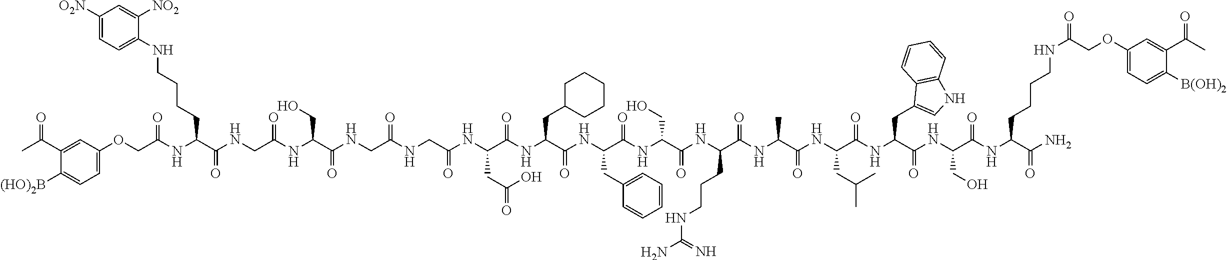 [SMILES: CC(=O)c1cc(OCC(=O)NCCCC[C@H](NC(=O)[C@H](CO)NC(=O)[C@H](Cc2c[nH]c3ccccc23)NC(=O)[C@H](CC(C)C)NC(=O)[C@H](C)NC(=O)[C@@H](CCCNC(=N)N)NC(=O)[C@@H](CO)NC(=O)[C@H](Cc2ccccc2)NC(=O)[C@H](CC2CCCCC2)NC(=O)[C@H](CC(=O)O)NC(=O)CNC(=O)CNC(=O)[C@H](CO)NC(=O)CNC(=O)[C@H](CCCCNc2ccc([N+](=O)[O-])cc2[N+](=O)[O-])NC(=O)COc2ccc(B(O)O)c(C(C)=O)c2)C(N)=O)ccc1B(O)O] |
| | rCGM-1 |
| I-12 | 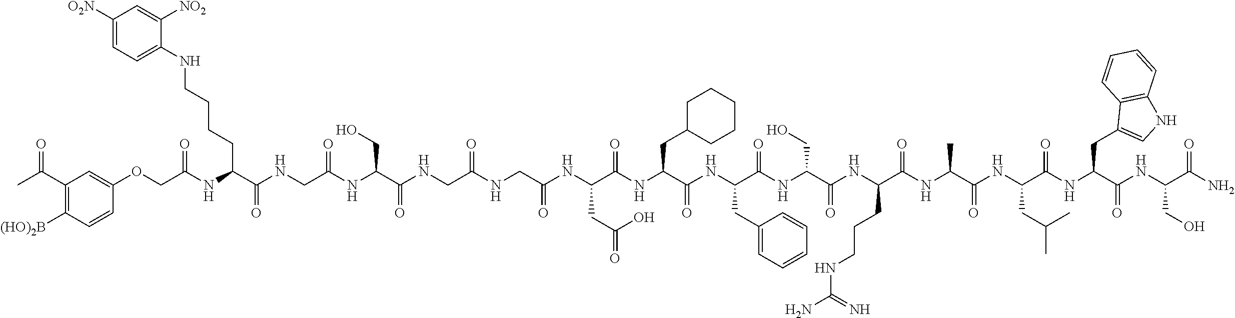 [SMILES: CC(=O)c1cc(OCC(=O)N[C@@H](CCCCNc2ccc([N+](=O)[O-])cc2[N+](=O)[O-])C(=O)NCC(=O)N[C@@H](CO)C(=O)NCC(=O)NCC(=O)N[C@@H](CC(=O)O)C(=O)N[C@@H](CC2CCCCC2)C(=O)N[C@@H](Cc2ccccc2)C(=O)N[C@H](CO)C(=O)N[C@H](CCCNC(=N)N)C(=O)N[C@@H](C)C(=O)N[C@@H](CC(C)C)C(=O)N[C@@H](Cc2c[nH]c3ccccc23)C(=O)N[C@@H](CO)C(N)=O)ccc1B(O)O] |
| | rCGM-2 |
| I-13 | 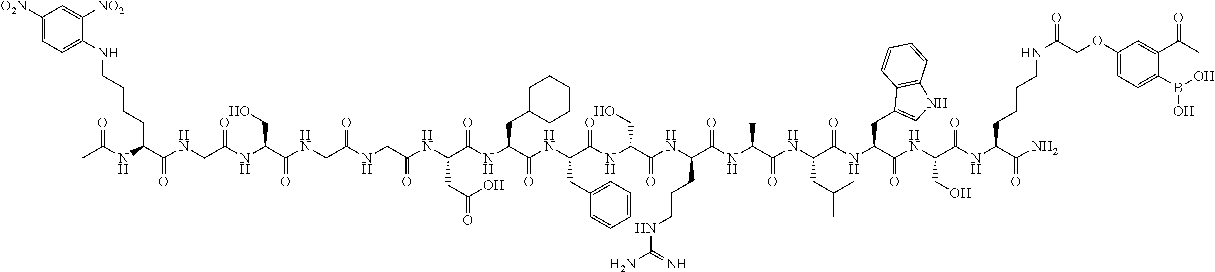 [SMILES: CC(=O)N[C@@H](CCCCNc1ccc([N+](=O)[O-])cc1[N+](=O)[O-])C(=O)NCC(=O)N[C@@H](CO)C(=O)NCC(=O)NCC(=O)N[C@@H](CC(=O)O)C(=O)N[C@@H](CC1CCCCC1)C(=O)N[C@@H](Cc1ccccc1)C(=O)N[C@H](CO)C(=O)N[C@H](CCCNC(=N)N)C(=O)N[C@@H](C)C(=O)N[C@@H](CC(C)C)C(=O)N[C@@H](Cc1c[nH]c2ccccc12)C(=O)N[C@@H](CO)C(=O)N[C@@H](CCCCNC(=O)COc1ccc(B(O)O)c(C(C)=O)c1)C(N)=O] |
| | rCGM-3 |
| | or a pharmaceutically acceptable salt and/or solvate thereof. |

In some embodiments, the pharmaceutically acceptable salt is an acid addition salt or a base addition salt. In some embodiments. for pharmaceutical methods and uses on human or animal subjects, the salt is a pharmaceutically acceptable salt. The selection of a suitable salt may be made by a person skilled in the art. Suitable salts include acid addition salts that may, for example, be formed by mixing a solution of a compound with a solution of a pharmaceutically acceptable acid such as hydrochloric acid, sulfuric acid, acetic acid, trifluoroacetic acid, or benzoic acid. Additionally, acids that are generally considered suitable for the formation of pharmaceutically useful salts from basic pharmaceutical compounds are discussed, for example, by P. Stahl et al, Camille G. (eds.) and Handbook of Pharmaceutical Salts. Properties, Selection and Use. (2002) Zurich. Wiley VCH; S. Berge et al, Journal of Pharmaceutical Sciences 1977 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In some embodiments, exemplary acid addition salts also include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates (“mesylates”), naphthalenesulfonates, nitrates, oxalates, phosphates, propionates, salicylates, succinates, sulfates, tartarates, thiocyanates, toluenesulfonates (also known as tosylates) and the like. In some embodiments, the mono- or di-acid salts are formed and such salts exist in either a hydrated, solvated or substantially anhydrous form. In general, acid addition salts are more soluble in water and various hydrophilic organic solvents and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Exemplary organic bases are isopropylamine, diethylamine, ethanolamine, trimethylamine, dicyclohexylamine, choline and caffeine. The selection of the appropriate salt may be useful, for example, so that an ester functionality, if any, elsewhere in a compound is not hydrolyzed. The selection criteria for the appropriate salt will be known to one skilled in the art. In some embodiments, exemplary basic salts also include ammonium salts, alkali metal salts such as sodium, lithium and potassium salts, alkaline earth metal salts such as calcium and magnesium salts, salts with organic bases (for example, organic amines) such as dicyclohexylamine, Abutyl amine, choline and salts with amino acids such as arginine, lysine and the like. Basic nitrogen containing groups may be quarternized with agents such as lower alkyl halides (e.g., methyl, ethyl and butyl chlorides, bromides and iodides), dialkyl sulfates (e.g., dimethyl, diethyl and dibutyl sulfates), long chain halides (e.g., decyl, lauryl and stearyl chlorides, bromides and iodides), aralkyl halides (e.g., benzyl and phenethyl bromides) and others. Compounds carrying an acidic moiety can be mixed with suitable pharmaceutically acceptable salts to provide, for example, alkali metal salts (e.g., sodium or potassium salts), alkaline earth metal salts (e.g., calcium or magnesium salts) and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

All such acid salts and base salts are intended to be pharmaceutically acceptable salts within the scope of the application and all acid and base salts are considered equivalent to the free forms of the corresponding compounds for purposes of the application. In addition, when a compound of the application contains both a basic moiety, such as, but not limited to an aliphatic primary, secondary, tertiary or cyclic amine, an aromatic or heteroaryl amine, pyridine or imidazole and an acidic moiety, such as, but not limited to tetrazole or carboxylic acid, zwitterions (“inner salts”) may be formed and are included within the terms “salt(s)” as used herein. It is understood that certain compounds of the application may exist in zwitterionic form, having both anionic and cationic centers within the same compound and a net neutral charge. Such zwitterions are included within the application.

Solvates of compounds of the application include, for example, those made with solvents that are pharmaceutically acceptable. Examples of such solvents include water (resulting solvate is called a hydrate) and ethanol and the like. Suitable solvents are physiologically tolerable at the dosage administered.

It is understood and appreciated that in some embodiments, compounds of the present application may have at least one chiral center and therefore can exist as enantiomers and/or diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

In some embodiments, the compounds of the present application can also include tautomeric forms, such as keto-enol tautomers and the like. Tautomeric forms can be in equilibrium or sterically locked into one form by appropriate substitution. It is intended that any tautomeric forms which the compounds form, as well as mixtures thereof, are included within the scope of the present application.

The compounds of the present application may further exist in varying amorphous and polymorphic forms and it is contemplated that any amorphous forms, polymorphs, or mixtures thereof, which form are included within the scope of the present application.

The compounds of the present application may further be radiolabeled and accordingly all radiolabeled versions of the compounds of the application are included within the scope of the present application. The compounds of the application also include those in which one or more radioactive atoms are incorporated within their structure.

The compounds of the present application may also comprise alternate isotopes of the atoms comprised therein. For example, one or more of available hydrogen atoms are independently replaced with deuterium.

### Compositions

The compounds of the present application are suitably formulated in a conventional manner into compositions using one or more carriers. Accordingly, the present application also includes a composition comprising one or more compounds of the application and a carrier. The compounds of the application are suitably formulated into pharmaceutical compositions for administration to subjects in a biologically compatible form suitable for administration in vivo. Accordingly, the present application further includes a pharmaceutical composition comprising one or more compounds of the application and a pharmaceutically acceptable carrier. In embodiments of the application the pharmaceutical compositions are used in the treatment of any of the diseases, disorders or conditions described herein.

The compounds of the application are administered to a subject in a variety of forms depending on the selected route of administration, as will be understood by those skilled in the art. For example, a compound of the application is administered by oral, inhalation, parenteral, buccal, sublingual, nasal, rectal, vaginal, patch, pump, topical or transdermal administration and the pharmaceutical compositions formulated accordingly. In some embodiments, administration is by means of a pump for periodic or continuous delivery. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

Parenteral administration includes systemic delivery routes other than the gastrointestinal (GI) tract, and includes, for example intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

In some embodiments, a compound of the application is orally administered, for example, with an inert diluent or with an assimilable edible carrier, or it is enclosed in hard or soft shell gelatin capsules, or it is compressed into tablets, or it is incorporated directly with the food of the diet. In some embodiments, the compound is incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). In embodiments, the tablets are coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions are formulated, for example as liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. In some embodiments, liposomes are formed from a variety of phospholipids, such as cholesterol, stearylamine or phosphatidylcholines. For oral administration in a capsule form, useful carriers or diluents include lactose and dried corn starch.

In some embodiments, liquid preparations for oral administration take the form of, for example, solutions, syrups or suspensions, or they are suitably presented as a dry product for constitution with water or other suitable vehicle before use. When aqueous suspensions and/or emulsions are administered orally, the compound of the application is suitably suspended or dissolved in an oily phase that is combined with emulsifying and/or suspending agents. If desired, certain sweetening and/or flavoring and/or coloring agents are added. Such liquid preparations for oral administration are prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of the application and use the lyophilizates obtained, for example, for the preparation of products for injection.

In some embodiments, a compound of the application is administered parenterally. For example, solutions of a compound of the application are prepared in water suitably mixed with a surfactant such as hydroxypropylcellulose. In some embodiments, dispersions are prepared in glycerol, liquid polyethylene glycols, dimethyl sulfoxide (DMSO) and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the compounds of the application are usually prepared, and the pH's of the solutions are suitably adjusted and buffered. For intravenous use, the total concentration of solutes should be controlled to render the preparation isotonic. For ocular administration, ointments or droppable liquids are delivered, for example, by ocular delivery systems known to the art such as applicators or eye droppers. In some embodiment, such compositions include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

In some embodiments, a compound of the application is formulated for parenteral administration by injection, including using conventional catheterization techniques or infusion. Formulations for injection are, for example, presented in unit dosage form, e.g., in ampoules or in multi-dose containers, with an added preservative. In some embodiments, the compositions take such forms as sterile suspensions, solutions or emulsions in oily or aqueous vehicles, and contain formulating agents such as suspending, stabilizing and/or dispersing agents. In all cases, the form must be sterile and must be fluid to the extent that easy syringability exists. Alternatively, the compounds of the application are suitably in a sterile powder form for reconstitution with a suitable vehicle, e.g., sterile pyrogen-free water, before use.

In some embodiments, compositions for nasal administration are conveniently formulated as aerosols, drops, gels and powders. For intranasal administration or administration by inhalation, the compounds of the application are conveniently delivered in the form of a solution, dry powder formulation or suspension from a pump spray container that is squeezed or pumped by the subject or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which, for example, take the form of a cartridge or refill for use with an atomising device. Alternatively, the sealed container is a unitary dispensing device such as a single dose nasal inhaler or an aerosol dispenser fitted with a metering valve which is intended for disposal after use. Where the dosage form comprises an aerosol dispenser, it will contain a propellant which is, for example, a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. In some embodiments, the pressurized container or nebulizer contains a solution or suspension of the active compound. Capsules and cartridges (made, for example, from gelatin) for use in an inhaler or insufflator are, for example, formulated containing a powder mix of a compound of the application and a suitable powder base such as lactose or starch. The aerosol dosage forms can also take the form of a pump-atomizer.

Compositions suitable for buccal or sublingual administration include tablets, lozenges, and pastilles, wherein a compound of the application is formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of the application are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, PA, 1980, pp. 1530-1533 for further discussion of suppository dosage forms.

In some embodiments a compound of the application is coupled with soluble polymers as targetable drug carriers. Such polymers include, for example, polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, in some embodiments, a compound of the application is coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

A compound of the application including pharmaceutically acceptable salts, solvates and/or prodrugs thereof is suitably used on their own but will generally be administered in the form of a pharmaceutical composition in which the one or more compounds of the application (the active ingredient) is in association with a pharmaceutically acceptable carrier. Depending on the mode of administration, the pharmaceutical composition will comprise from about 0.05 wt % to about 99 wt % or about 0.10 wt % to about 70 wt %, of the active ingredient, and from about 1 wt % to about 99.95 wt % or about 30 wt % to about 99.90 wt % of a pharmaceutically acceptable carrier, all percentages by weight being based on the total composition.

In the above, the term “a compound” also includes embodiments wherein one or more compounds are referenced.

### Target Proteins

In some embodiments, at least one of the first and second target proteins is an immune cell receptor. In some embodiments, the immune cell is selected from monocytes, macrophages, polymorphonuclear cells, erythrocytes, megakaryocytes, neutrophils, basophils, eosinophils, dendritic cells, natural killer (NK) cells B cells, lymphocytes and T cells. In some embodiments, the immune cell is a T cell. In some embodiments, the immune cell is an engineered T cell. In some embodiments, the engineered T cell comprises a synthetic antigen receptor (SAR).

In some embodiments, the first target protein is an antibody and the second target protein is a cell surface receptor. In some embodiments, the first target protein is an antibody and the second target protein is a tumor antigen. In some embodiments, PBD1 comprises a hapten, including, without limitation, the haptens disclosed herein, that selectively binds to an antibody comprising a hapten binding site and PBD2 comprises a cell surface receptor binding domain. In some embodiments, PBD1 comprises a hapten that selectively binds to an antibody comprising a hapten binding site and PBD2 comprises a tumor antigen binding domain.

In some embodiments, the first and second target proteins are each a cell surface receptor. In some embodiments, PBD1 selectively binds to an immune cell receptor and PBD2 selectively binds to a cell surface receptor. In some embodiments, PBD1 selectively binds to an immune cell receptor and PBD2 selectively binds to a tumor antigen.

In some embodiments, the first and second target proteins are each independently selected from an urokinase plasminogen activating receptor (uPAR), a prostate-specific membrane antigen (PSMA), a human epidermal growth factor receptor 2 (HER2), an integrin, CD38, programmed death-ligand-1 (PD-L1), a G protein-coupled receptor (GPCR), a Kirsten rat sarcoma virus (KRAS), a vascular endothelial growth factor (VEGF) and a folate receptor.

### III. Methods and Uses of the Application

The compounds of the application, herein defined as proximity inducing molecules, have been shown to covalently bind to the target proteins and reversibly link the target proteins and, in some embodiments, target cells.

Accordingly, in some embodiments, the present application includes a method for forming ternary protein complexes, either in a biological sample or in a subject, comprising administering an effective amount of a compound or composition of the application to the biological sample or subject. Also provided is use of a compound or composition of the application for forming ternary protein complexes in a biological sample or in a subject. Further provided is a compound or composition of the application for use in forming ternary protein complexes in a biological sample or in a subject. In some embodiments, the reversibility may release the immune cells from the target cell which should increase the processivity of the immune cell attack. In some embodiments, the reversibility may allow for correction of potential off target labeling, favoring on desired target labeling, thus overall enhancing selectivity by minimizing off-target effects while maximizing desired interactions.

In some embodiments, administering or using the compound or composition of the application comprises first exposing the compound or composition to the first target protein ex vivo, so that the PBD1 binds its target protein and a covalent bond between the target protein and the compound of the application is formed and then administering the resulting complex to the subject, whereby the resulting complex then binds via the PBD2 to its target in the biological sample or subject. In some embodiments, the use of the compound or composition of the application comprises use of the compound or composition covalently bound to the first target protein via the PBD1, whereby upon use the resulting complex then binds via the PBD2 to its target in the biological sample or subject.

A person skilled in the art can readily choose the first and second PBD depending on the intended benefit of bringing the two proteins in proximity of each other. As described herein, target proteins and their PBDs are known and the selection of suitable PBDs for a particular therapeutic use can be made by a person skilled in the art.

Accordingly, also included is a method for recruiting an antibody or an immune cell for immunotherapy, either in a biological sample or in a subject, comprising administering an effective amount of a compound or composition of the application to the biological sample or subject, wherein the compound or composition comprises PBD1, which selectively binds to the antibody or immune cell and PBD2, which selectively binds to the target, such as an antigen on a cancer cell. Also included is a use of a compound or composition of the application for recruiting an antibody or an immune cell for immunotherapy in a biological sample or subject in need thereof. Further provided is a use of a compound or composition of the application in the manufacture of a medicament for immunotherapy in a biological sample or subject in need thereof. Even further provided is a compound or composition of the application in the for use in immunotherapy in a biological sample or subject in need thereof.

Further included is a method for recruiting an antibody or an immune cell and targeting a cell for provoking an immune response to the cell, either in a biological sample or in a subject, comprising administering an effective amount of a compound or composition of the application to the biological sample or the subject, wherein PBD1 selectively binds the antibody or a protein on the surface of the immune cell and PBD2 selectively binds a protein on the surface of the cell. Further provided is use of a compound or composition of the application for provoking an immune response to a cell in a biological sample or subject in need thereof, wherein PBD1 selectively binds the antibody or a protein on the surface of the immune cell and PBD2 selectively binds a protein on the surface of the cell. Also provided is use of a compound or composition of the application in the manufacture of a medicament for provoking an immune response to a cell in a biological sample or subject in need thereof, wherein PBD1 selectively binds the antibody or a protein on the surface of the immune cell and PBD2 selectively binds a protein on the surface of the cell. Even further provided is a compound or composition of the application for use in provoking an immune response to a cell in a biological sample or subject in need thereof, wherein PBD1 selectively binds the antibody or a protein on the surface of the immune cell and PBD2 selectively binds a protein on the surface of the cell. In some embodiments, the immune response comprises targeted killing of the cell or phagocytosis of the target cell. In some embodiments, the protein on the surface of the cell comprises a tumor antigen on the surface of a cancer cell and the immune response comprises targeted killing of the cancer cell.

The present application also includes a method of treating a disease, disorder, or condition that is treatable by engaging an immune response or by immunotherapy, comprising administering a therapeutically effective amount of a compound or composition of the application to a subject in need thereof, wherein the PBD1 selectively binds to an antibody or immune cell and PBD2 selectively binds to a particular cell, structure organ affected by the disease, disorder or condition. Also provided is use of a compound or composition of the application for treating a disease, disorder or condition that is treatable by engaging an immune response in a subject in need thereof, wherein the PBD1 selectively binds to an antibody or immune cell and PBD2 selectively binds to a particular cell, structure organ affected by the disease, disorder or condition. Further provided is use of a compound or composition of the application in the manufacture of a medicament for treating a disease, disorder or condition that is treatable by engaging an immune response in a subject in need thereof, wherein the PBD1 selectively binds to an antibody or immune cell and PBD2 selectively binds to a particular cell, structure organ affected by the disease, disorder or condition. Even further provided is a compound or composition of the application for use in treating a disease, disorder or condition that is treatable by engaging an immune response in a subject in need thereof, wherein the PBD1 selectively binds to an antibody or immune cell and PBD2 selectively binds to a particular cell, structure organ affected by the disease, disorder or condition. Such diseases, disorders, or conditions that are treatable by immunotherapy or by engaging an immune response include, without limitation, cancer, autoimmune diseases, and allergy, and transplant rejection.

In some embodiments, the compounds of the application bind to and recruit antibodies, such as endogenous antibodies, to the surface of target cells such that the resulting complex comprises the antibody and a protein on the surface of the target cell. These ternary complexes bind activation receptors on immune cells (e.g. CD64 on monocytes, CD3 receptors on T-cells and CD16u receptors on NK cells). The result is the activation of endogenous T cell or NK cell cytotoxicity against the target cell or other immune response provocation. Accordingly, compounds of the application are also effective tools for triggering a cytotoxic response to target cells.

In some embodiments, functionalized cells are generated by contacting immune cells expressing an FcR with a suitable compound of the application, wherein the compound comprises a PBD1 that selectively binds to the FcR, and a PBD2 that selectively binds to a cell of interest to be targeted, wherein the generated functionalized cell is covalently bound to the PBD2. As will be understood, the suitability of a PBD2 will depend on the intended application or use of the functionalized cell. For example, where the intended application of the functionalized cell is for the treatment of a specific cancer, a PBD2 will be one that binds cells of the specific cancer. The skilled person can readily select a suitable PBD2 for the intended application or use.

Accordingly, an aspect described herein includes a method for generating a functionalized cell, the method comprising providing a cell expressing an FcR, and contacting the cell with a compound of the application (comprising a PBD1 that interacts with the FcR under suitable conditions to allow binding of the PBD1 and FcR, and covalent attachment of the covalent labeling domain (CLD) to the FcR, thereby generating a functionalized cell.

Functionalized cells may be generated for example in vitro, ex vivo, or in vivo. For example, an immune cell is contacted in vitro or ex vivo with a compound of the application. Alternatively, the compound of the application is administered to a subject, and the functionalized cell generated in vivo.

The functionalized cells described herein may be directed towards a target cell and exhibit a cytotoxic response to, or phagocytosis of, the target cell. Accordingly, the functionalized cells described herein are effective tools for directing the functionalized cell to a target cell, and/or triggering a cytotoxic response to, or phagocytosis of, the target cell. The target cell is any desired cell. For example, in the context of cancer therapy, the target cell is a cancer cell.

Also described herein is a method for recruiting a functionalized cell described herein to a target cell in a subject, comprising administering an effective amount of a) a compound or composition of the application; or b) a functionalized cell to the subject. Also provided is use of a) a compound or composition of the application; or b) a functionalized cell described herein for recruiting a functionalized cell to a target cell in a subject in need thereof. Further provided is use of a) a compound or composition of the application; or b) a functionalized cell described herein in the manufacture of a medicament for recruiting a functionalized cell to a target cell in a subject in need thereof. Even further provided is a) a compound or composition of the application; or b) a functionalized cell described herein for use in recruiting a functionalized cell to a target cell in a subject in need thereof. In some embodiments, the functionalized cell provokes an immune response to the target cell, such as a cytotoxic or phagocytic response.

Further described herein is a method for targeting and/or recruiting a functionalized cell for provoking an immune response to a target cell in a subject, comprising administering an effective amount of a) a compound or composition of the application, comprising at least one suitable PBD; or b) a functionalized cell comprising at least one suitable PBD to the subject. An aspect also includes use of a) a compound or composition of the application, comprising at least one suitable PBD; or b) a functionalized cell comprising at least one suitable PBD for provoking an immune response to a target cell in a subject. An aspect also includes use of a) a compound of the application, comprising at least one suitable PBD; or b) a functionalized cell comprising at least one suitable PBD in the manufacture of a medicament for provoking an immune response to a target cell in a subject. An aspect also includes a) a compound or composition of the application, comprising at least one suitable PBD; or b) a functionalized cell comprising at least one suitable PBD for use in provoking an immune response to a target cell in a subject.

In some embodiments, the present application includes a method of treating a disease, disorder or condition that is treatable by provoking an immune response, comprising administering a therapeutically effective amount of a compound of the application, to a subject in need thereof.

In some embodiments, the present application includes a use of a compound of the application, for treating a disease, disorder or condition treatable by immunotherapy. The application also includes use of a compound of the application, for the preparation of a medicament for treating of a disease, disorder or condition treatable by immunotherapy.

In some embodiments, the disease, disorder or condition treatable by immunotherapy or by provoking an immune response is cancer, therefore the present application includes a method of treating cancer comprising administering a therapeutically effective amount of a compound or composition of the application to a subject in need thereof. The present application also includes a use of a compound or composition of the application for treatment of cancer as well as a use of a compound of the application for the preparation of a medicament for treatment of cancer. The application further includes a compound or composition of the application for use in treating cancer. In some embodiments, the compound or composition is administered for the prevention of cancer in a subject such as a mammal having a predisposition for cancer.

In some embodiments, the cancer is one that is impacted or treatable by immunotherapy. In some embodiments, the cancer is one that is impacted or treatable by activation of endogenous immune cells. In some embodiments, the cancer is one that is impacted or treatable by provoking an immune response to tumor cells. In some embodiments, the cancer is one that is impacted or treatable by provoking phagocytosis of tumor cells.

In some embodiments, the cancer is selected from, but not limited to: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-CeIl Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-CeIl; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor. Metastases of the aforementioned cancers can also be treated in accordance with the methods described herein.

In some embodiments, the cancer is selected from prostate cancer, breast cancer, ovarian cancer and glioblastoma. In some embodiments, the cancer is glioblastoma.

In further embodiments, the present application also includes a method of treating a disease, disorder or condition treatable by immunotherapy, comprising administering to a subject in need thereof a therapeutically effective amount of a compound or composition of the application in combination with another agent useful for treatment of the disease, disorder or condition treatable by immunotherapy. The present application also includes a use of a compound or composition of the application in combination with an agent useful for treatment of a disease, disorder or condition treatable by immunotherapy, for treatment of such disease, disorder or condition.

In some embodiments, the disease, disorder or condition treatable by immunotherapy is cancer and the compounds or composition of the application is administered in combination with one or more additional cancer treatments. In some embodiments, the additional cancer treatment is selected from radiotherapy, chemotherapy, targeted therapies such as antibody therapies and small molecule therapies such as tyrosine-kinase and serine-threonine kinase inhibitors, immunotherapy, hormonal therapy and anti-angiogenic therapies.

In some embodiments, effective amounts vary according to factors such as the disease state, age, sex and/or weight of the subject. In a further embodiment, the amount of a given compound or compounds that will correspond to an effective amount will vary depending upon factors, such as the given drug(s) or compound(s), the pharmaceutical formulation, the route of administration, the type of condition, disease or disorder, the identity of the subject being treated, and the like, but can nevertheless be routinely determined by one skilled in the art.

In some embodiments, the compound or composition of the application is administered at least once a week. However, in some embodiments, the compound or composition is administered to the subject from about one time per two weeks, three weeks or one month. In some embodiments, the compound or composition is administered about one time per week to about once daily. In another embodiment, the compound or composition is administered 2, 3, 4, 5 or 6 times daily. The length of the treatment period depends on a variety of factors, such as the severity of the disease, disorder or condition, the age of the subject, the concentration and/or the activity of the compound of the application, and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration is required. For example, the compounds or compositions are administered to the subject in an amount and for duration sufficient to treat the subject.

In some embodiments, the subject is a mammal. In another embodiment, the subject is human.

A compound or composition of the application is either used alone or in combination with other known agents useful for treating diseases, disorders or conditions as defined above, such as the compounds disclosed herein. When used in combination with other agents useful in treating such diseases, disorders or conditions, it is some embodiments that a compound or composition of the application is administered contemporaneously with those agents. As used herein, “contemporaneous administration” of two substances to a subject means providing each of the two substances so that they are both active in the individual at the same time. The exact details of the administration will depend on the pharmacokinetics of the two substances in the presence of each other, and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. It is a further embodiment of the present application that a combination of agents is administered to a subject in a non-contemporaneous fashion. In some embodiments, a compound of the present application is administered with another therapeutic agent simultaneously or sequentially in separate unit dosage forms or together in a single unit dosage form. Accordingly, the present application provides a single unit dosage form comprising one or more compounds of the application, an additional therapeutic agent, and a pharmaceutically acceptable carrier.

The dosage of a compound of the application varies depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. In some embodiments, a compound of the application is administered initially in a suitable dosage that is adjusted as required, depending on the clinical response. Dosages will generally be selected to maintain a serum level of the compound of the application from about 0.01 μg/cc to about 1000 μg/cc, or about 0.1 μg/cc to about 100 μg/cc. As a representative example, oral dosages of one or more compounds of the application will range between about 1 mg per day to about 1000 mg per day for an adult, suitably about 1 mg per day to about 500 mg per day, more suitably about 1 mg per day to about 200 mg per day. For parenteral administration, a representative amount is from about 0.001 mg/kg to about 10 mg/kg, about 0.01 mg/kg to about 10 mg/kg, about 0.01 mg/kg to about 1 mg/kg or about 0.1 mg/kg to about 1 mg/kg will be administered. For oral administration, a representative amount is from about 0.001 mg/kg to about 10 mg/kg, about 0.1 mg/kg to about 10 mg/kg, about 0.01 mg/kg to about 1 mg/kg or about 0.1 mg/kg to about 1 mg/kg. For administration in suppository form, a representative amount is from about 0.1 mg/kg to about 10 mg/kg or about 0.1 mg/kg to about 1 mg/kg.

In the above-described methods and uses, the term “a compound” also includes embodiments wherein one or more compounds are referenced.

### Methods of Preparation

The compounds of the application can be synthesized by any of the techniques that are know n to those skilled in the art. For example some compounds are amendable to standard polypeptide synthetic chemistry techniques, such as a solid-phase Merrifield-type synthesis. A summaiy of the many such techniques available can be found in Steward et al., “Solid Phase Peptide Synthesis”, W. Hf. Freeman Co., San Francisco, 1969; Bodanszky, et al., “Peptide Synthesis”, John Wiley & Sons, Second Edition, 1976; J. Meienhofer, “Hormonal Proteins and Peptides”, Vol. 2, p. 46, Academic Press (New York), 1983; Merrifield, Adv. Enzymol., 32:221-96, 1969; Fields et al., int. J. Peptide Protein Res., 35:161-214, 1990; and U.S. Pat. No. 4,244,946 for solid phase peptide synthesis, and Schroder et al., “The Peptides”, Vol. 1, Academic Press (New York), 1965 for classical solution synthesis, each of which is incorporated herein by reference. Appropriate protective groups usable in such synthesis are described in the above texts and in J. F. W. McOmie, “Protective Groups in Organic Chemnisty”, Plenum Press, New York, 1973, which is incorporated herein by reference.

In general, the solid-phase synthesis methods contemplated comprise the sequential addition of one or more amino acid residues or suitably protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group such as lysine.

Using a solid phase synthesis as an example, the protected or derivatized amino acid can be attached to an inert solid support through its unprotected carboxyl or amino group. The protecting group of the amino or carboxyl group can then be selectively removed and the next amino acid in the sequence having the complimentary (amino or carboxyl) group suitably protected is admixed and reacted under conditions suitable for forming the amide linkage with the residue already attached to the solid support. The protecting group of the amino or carboxyl group can then be removed from this newly added amino acid residue, and the next amino acid (suitably protected) is then added, and so forth. After all the desired amino acids have been linked in the proper sequence, any remaining terminal and side group protecting groups (and solid support) can be removed sequentially or concurrently, to afford the final linear polypeptide.

The formation of a desired compound salt is achieved using standard techniques. For example, the neutral compound is treated with an acid or base in a suitable solvent and the formed salt is isolated by filtration, extraction or any other suitable method.

The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

Specific enantiomers or diastereomers of the compounds of the application are available by using corresponding single enantiomers or diastereomers of the corresponding starting materials.

### EXAMPLES

The following non-limiting examples are illustrative of the present application:

### General Synthesis Methods:

All reagents and solvents were obtained from commercial suppliers (Sigma Aldrich, BroadPharm, Conju-Probe) and were used without purification. Thin layer chromatography (TLC) was performed using aluminum silica gel sheets (Silicycle™) and visualized with fluorescence (254 nm) and ninhydrin staining. Dibenzocylcooctyne-N-hydroxysuccinimidyl ester (DBCO-NHS) was generously provided by Prof Alex Adronov (McMaster University). Milli-Q water was purified using a Milli-Q® EQ 7000 Ultrapure Water Purification System (Millipore, Cat. No. C228480). Column chromatography was conducted using a Buchi™ Pure C-810 Flash system using silica or C-18 columns from Buchi. ¹H NMR spectra were recorded in deuterated solvents as indicated on a Bruker™ 700 MHz spectrometer. Liquid chromatography-mass spectrometry (LC-MS) was performed using an Agilent™ Infinity C-18 column coupled to a Thermo LTQ Orbitrap XL system. Preparative high performance liquid chromatography (HPLC) was performed with a ThermoFisher DIONEX™ UltiMate 3000 UHPLC+system with a Hypersil GOLD 150 mm×10 mm C-18 column, and a gradient of 5:95 to 95:5 acetonitrile:water with 0.1% formic acid.

### Synthesis of Int-1

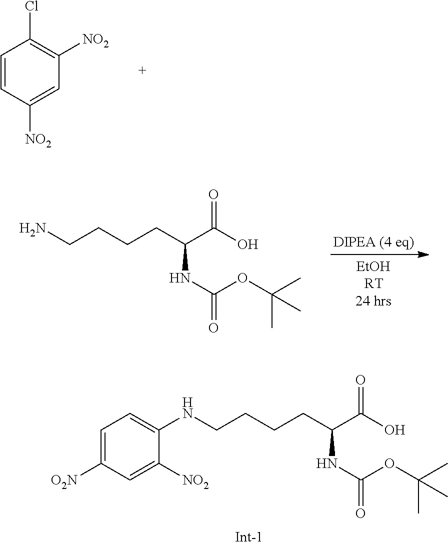 [SMILES: O=[N+]([O-])c1ccc(Cl)c([N+](=O)[O-])c1 ; CC(C)(C)OC(=O)N[C@@H](CCCCN)C(=O)O ; CC(C)(C)OC(=O)N[C@@H](CCCCNc1ccc([N+](=O)[O-])cc1[N+](=O)[O-])C(=O)O]

A solution of 1-chloro-2,4-dinitrobenzene (328 mg, 1.62 mmol, 2 eq), Lys(Boc)-OH (200 mg, 0.812 mmol, 1 eq) and N,N-diisopropylethylamine (0.6 mL, 3.25 mmol, 4 eq) was stirred in 4 mL of ethanol at room temperature overnight. Excess Boc-Lys was removed through gravity filtration, and the solvent was removed in vacuo. The crude material was purified by normal phase silica column chromatography (95:5 dichloromethane (DCM): methanol (MeOH)) to yield Int-1 as a yellow solid (217.1 mg, 53.3% yield). ¹H NMR (700 MHz, CDCl₃) δ 9.12 (d, J=2.6 Hz, 1H), 8.55 (d, J=5.5 Hz, 1H), 8.26 (dd, J=9.5, 2.6 Hz, 1H), 6.94 (d, J=9.5 Hz, 1H), 4.33-4.19 (m, 1H), 3.41 (q, J=6.7 Hz, 2H), 3.10 (q, J=7.4 Hz, 2H), 1.94 (s, 1H), 1.87-1.69 (m, 3H), 1.59-1.54 (m, 2H), 1.42 (s, 9H).

### Synthesis of Int-2A

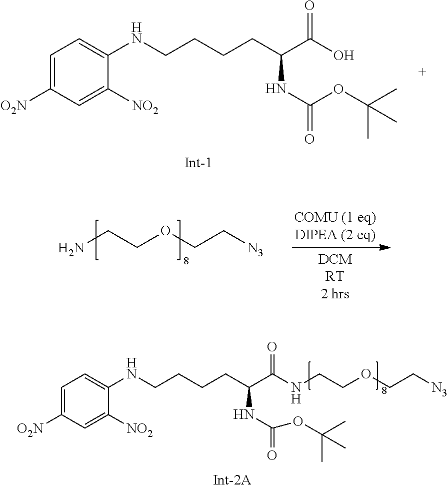

Int-1 (82.5 mg, 0.2 mmol, 1 eq) was dissolved in 1 mL of DCM and combined with amino-PEG₈-azide (87.7 mg, 0.2 mmol, 1 eq). N,N-diisopropylethylamine (0.072 mL, 0.4 mmol, 2 eq) was added, followed by (1-Cyano-2-ethoxy-2-oxoethylidenaminooxy)dimethylamino-morpholino-carbenium hexafluorophosphate (COMU) (85.7 mg, 0.2 mmol, 1 eq). The solution was stirred at room temperature for 2 hrs. Solvent was removed in vacuo, and the crude material was purified with reverse phase chromatography with a C18 silica column (5:95 to 95:5 acetonitrile:water gradient) to yield Int-2A as a yellow oil. (93 mg, 55.9% yield). ¹H NMR (700 MHz, CDCl₃) δ 9.11 (d, J=2.7 Hz, 1H), 8.53 (t, J=4.9 Hz, 1H), 8.25 (dd, J=9.5, 2.4 Hz, 1H), 7.26 (s, 1H), 6.92 (d, J=9.5 Hz, 1H), 6.87 (t, J=5.2 Hz, 1H), 5.30-5.26 (m, 1H), 4.12 (d, J=5.4 Hz, 1H), 3.67-3.57 (m, 34H), 3.56-3.52 (m, 2H), 3.48-3.34 (m, 7H), 1.92-1.85 (m, 1H), 1.78 (tdt, J=20.8, 13.7, 7.0 Hz, 2H), 1.69-1.61 (m, 1H), 1.55-1.46 (m, 2H), 1.41 (s, J=9.2 Hz, 10H). LC-HRMS [M+H]⁺ m/z calc for [C₃₅H₆₀N₈O₁₅]: 833.4256, found 833.0960.

### Synthesis of Int-2B

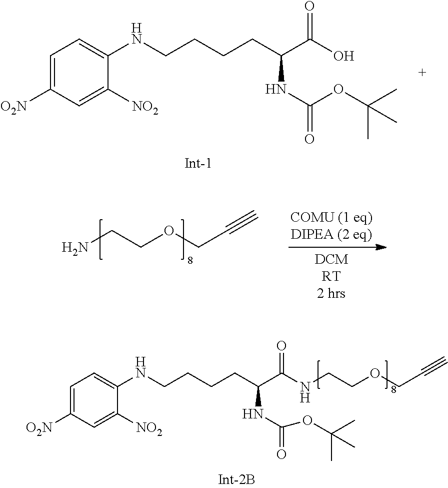

Int-1 (82.5 mg, 0.2 mmol, 1 eq) was dissolved in 1 mL of DCM and combined with amino-PEG₈-alkyne (82 mg, 0.2 mmol, 1 eq). N,N-diisopropylethylamine (0.072 mL, 0.4 mmol, 2 eq) was added, followed by COMU (86 mg, 0.2 mmol, 1 eq). The solution was stirred at room temperature for 2 hrs. Solvent was removed in vacuo, and the crude material was purified with reverse phase chromatography with a C18 silica column (5:95 to 95:5 acetonitrile:water gradient) to yield Int-2B as a yellow oil. (64.8 mg, 40% yield). ¹H NMR (700 MHz, CDCl₃) δ 9.08 (d, J=2.0 Hz, 1H), 8.52 (s, 1H), 8.23 (dd, J=9.4, 1.9 Hz, 1H), 8.10 (s, 1H), 7.26 (s, 1H), 6.91 (d, J=9.5 Hz, 1H), 6.78 (s, 1H), 5.31 (d, J=6.9 Hz, 1H), 4.16 (d, J=1.9 Hz, 2H), 4.11 (d, J=4.3 Hz, 1H), 3.69-3.55 (m, 32H), 3.53 (t, J=4.9 Hz, 2H), 3.47-3.35 (m, 5H), 2.41 (s, 1H), 1.87 (td, J=14.6, 6.1 Hz, 1H), 1.76 (ddd, J=28.2, 14.0, 6.6 Hz, 2H), 1.67-1.60 (m, 1H), 1.49 (d, J=6.0 Hz, 2H), 1.40 (s, 10H).

### Synthesis of Int-2C

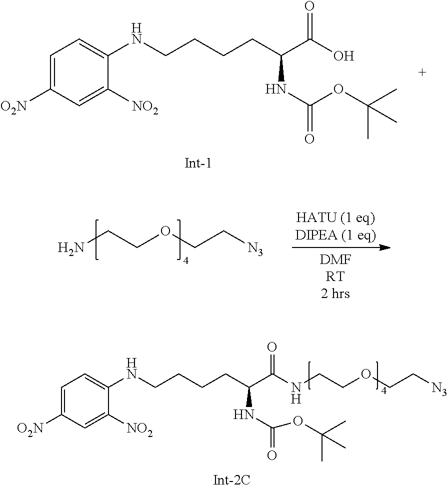

Int-I (82.5 mg, 0.2 mmol, 1 eq) was dissolved in 1 mL of DMF and combined with HATU (76.0 mg, 0.2 mmol, 1 eq) and N,N-diisopropylethylamine (25.9 mg, 0.2 mmol, 1 eq). The solution was stirred at room temperature for 30 minutes, followed by the addition of azido-PEG₄-amine (52.5 mg, 0.2 mmol, 1 eq). The reaction was stirred for 2 hours at room temperature. Solvent was removed in vacuo, and the crude material was purified with reverse phase chromatography with a C18 silica column (5:95 to 95:5 acetonitrile:water gradient) to yield Int-2C as a yellow oil. (32.9 mg, 25% yield).¹H NMR (700 MHz, CDCl₃) δ 9.12 (d, J=2.7 Hz, 1H), 8.54 (t, J=5.3 Hz, 1H), 8.26 (dd, J=9.5, 2.7 Hz, 1H), 6.92 (d, J=9.5 Hz, 1H), 6.78 (t, J=5.6 Hz, 1H), 5.24 (d, J=8.3 Hz, 1H), 4.12 (q, J=7.3 Hz, 1H), 3.70-3.58 (m, 14H), 3.55 (t, J=5.1 Hz, 2H), 3.50-3.35 (m, 6H), 2.61 (s, 1H), 1.93-1.84 (m, 1H), 1.84-1.71 (m, 2H), 1.70-1.61 (m, 1H), 1.56-1.46 (m, 3H), 1.42 (s, 9H). LC-HRMS [M+H]⁺ m/z calc for [C₂₇H₄₄N₈O₁₁]: 657.3208, found 657.2570.

### Synthesis of Int-3A

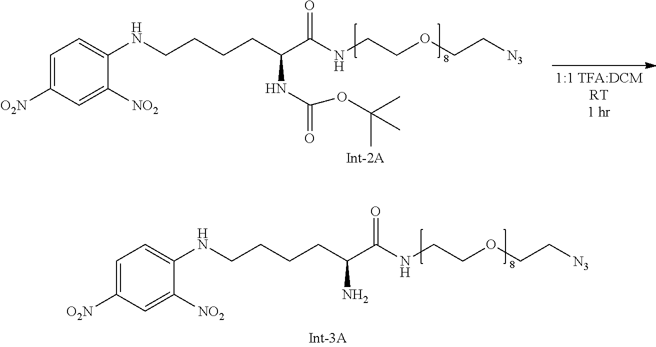

Int-2A (93 mg, 0.12 mmol, 1 eq) was dissolved in 2 mL of 1:1 trifluoroacetic acid (TFA):DCM and stirred for 1 hr at room temperature. Solvent was removed in vacuo and the crude product carried through to the next step.

### Synthesis of Int-3B

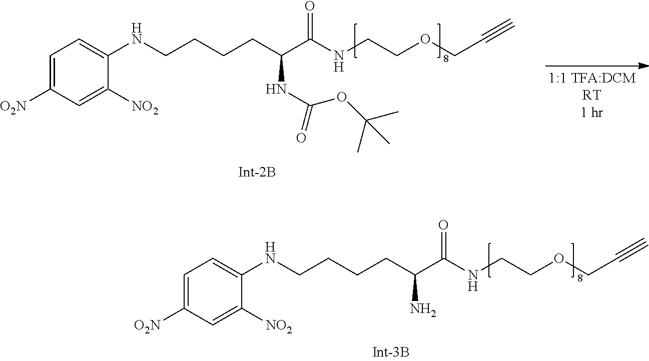

Int-2B (64.8 mg, 0.08 mmol, 1 eq) was dissolved in 2 mL of 1:1 TFA:DCM and stirred for 1 hr at room temperature. Solvent was removed in vacuo and the crude product carried through to the next step.

### Synthesis of Int-3C

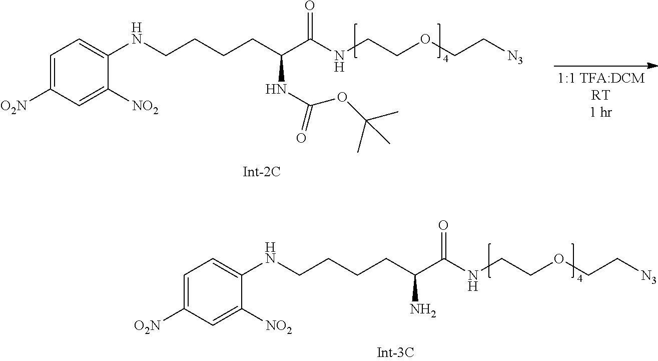

Int-2C (32.9 mg, 0.05 mmol, 1 eq) was dissolved in 2 mL of 1:1 TFA:DCM and stirred for 1 hr at room temperature. Solvent was removed in vacuo and the crude product carried through to the next step.

### Synthesis of Int-4A

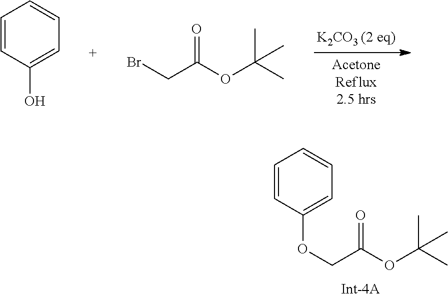

Phenol (94 mg, 1 mmol, 1 eq) was dissolved in 5 mL of acetone. Potassium carbonate (417 mg, 2 mmol, 2 eq) was added and the solution stirred at room temperature for 10 minutes. Next, tert-butyl bromoacetate (195 mg, 1 mmol, 1 eq) was added dropwise and the reaction brought to reflux for 2.5 hours. The solvent was then removed in vacuo, and the residue was redissolved in 15 mL of ethyl acetate and washed 3× with 15 mL of 1 M sodium hydroxide, followed by a final 15 mL brine wash. The organic layer was dried over sodium sulfate and concentrated to provide Int-4A as a yellow oil (98.2 mg, 47% yield). ¹H NMR (700 MHz, CDCl₃) δ 7.21-7.15 (m, 2H), 6.87 (tt, J=7.3, 1.0 Hz, 1H), 6.83-6.76 (m, 2H), 4.41 (s, 2H), 1.39 (s, 9H).

### Synthesis of Int-4C

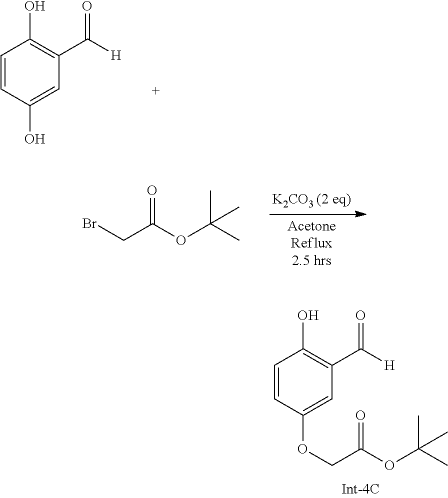

2,5-dihydroxybenzaldehyde (100 mg, 0.725 mmol, 1 eq) was dissolved in 5 mL of acetone. Potassium carbonate (200 mg, 1.45 mmol, 2 eq) was added and the solution stirred at room temperature for 10 minutes. Next, tert-butyl bromoacetate (141 mg, 0.724 mmol, 1 eq) was added dropwise and the reaction brought to reflux for 2.5 hours. The solution was then cooled and filtered. The filtrate was concentrated and purified with a silica column (0:100 to 100:0 ethyl acetate:hexanes) to provide Int-4C as a yellow oil (18.9 mg, 10% yield). ¹H NMR (700 MHz, CDCl₃) δ 10.39 (s, 1H), 7.19 (d, J=3.2 Hz, 1H), 7.03-6.90 (m, 1H), 6.68 (d, J=8.9 Hz, 1H), 6.37 (s, J=12.8 Hz, 1H), 4.53 (s, 2H), 1.42 (s, 9H).

### Synthesis of Int-4B

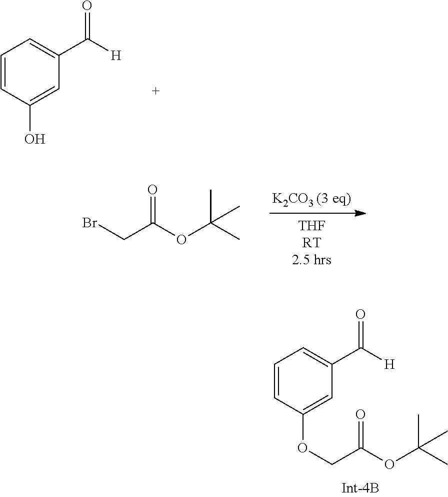

3-hydroxybenzaldehyde (122 mg, 1 mmol, 1 eq) was dissolved in 5 mL of tetrahydrofuran. Potassium carbonate (415 mg, 3 mmol, 3 eq) was added and the solution stirred at room temperature for 10 minutes. Next, tert-butyl bromoacetate (235 mg, 1.2 mmol, 1.2 eq) was added dropwise and the reaction stirred for 2.5 hours. The solution was then cooled and filtered. The filtrate was concentrated and purified with a silica column (0:100 to 100:0 ethyl acetate:hexanes) to provide Int-4B as a clear oil (105.7 mg, 45% yield). ¹H NMR (700 MHz, CDCl₃) δ 9.93 (s, 1H), 7.48-7.45 (m, 1H), 7.42 (t, J=7.8 Hz, 1H), 7.33-7.31 (m, 1H), 7.18 (dd, J=8.1, 2.7 Hz, 1H), 4.55 (s, 2H), 1.46 (d, J=1.3 Hz, 9H).

### Synthesis of Int-5A

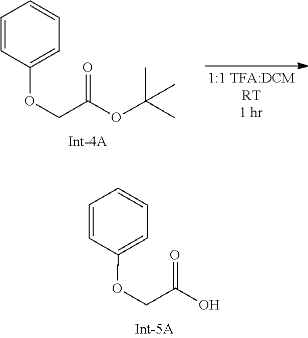

Int-4A (98.2 mg, 0.47 mmol, 1 eq) was dissolved in 2 mL of 1:1 TFA:DCM and stirred for 1 hr at room temperature. Solvent was removed in vacuo and the crude product carried through to the next step.

### Synthesis of Int-5C

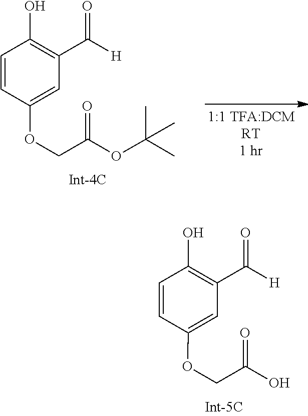 [SMILES: CC(C)(C)OC(=O)COc1ccc(O)c(C=O)c1 ; O=Cc1cc(OCC(=O)O)ccc1O]

Int-4C (18.9 mg, 0.0725 mmol, 1 eq) was dissolved in 2 mL of 1:1 TFA:DCM and stirred for 1 hr at room temperature. Solvent was removed in vacuo and the crude product carried through to the next step.

### Synthesis of Int-5B

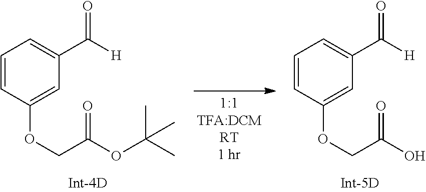 [SMILES: CC(C)(C)OC(=O)COc1cccc(C=O)c1 ; O=Cc1cccc(OCC(=O)O)c1]

Int-4B (105.7 mg, 0.45 mmol, 1 eq) was dissolved in 2 mL of 1:1 TFA:DCM and stirred for 1 hr at room temperature. Solvent was removed in vacuo and the crude product carried through to the next step.

### Synthesis of Int-4D

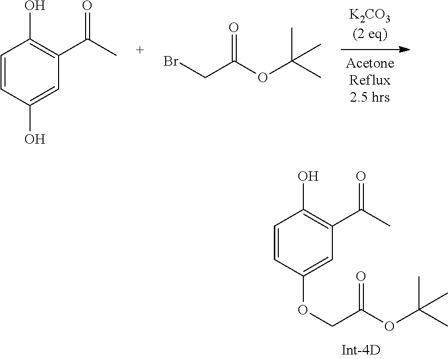

2,5-dihydroxyacetophenone (1.1 g, 7.25 mmol, 1 eq) was dissolved in 25 mL of acetone along with potassium carbonate (2 g, 14.5 mmol, 2 eq). Next, tert-butyl bromoacetate (1.4 g, 7.25 mmol, 1 eq) was added dropwise and the reaction stirred for 2.5 hours. The solution was then cooled and filtered. The filtrate was concentrated and purified with a silica column (0:100 to 100:0 ethyl acetate:hexanes) to provide Int-4D as a clear oil (1.02 g, 53% yield). ¹H NMR (700 MHz, CDCl₃) δ 11.85 (s, 1H), 7.26 (s, 1H), 7.22 (d, J=3.0 Hz, 1H), 7.11 (dd, J=9.1, 3.0 Hz, 1H), 6.91 (d, J=9.1 Hz, 1H), 4.48 (s, 2H), 2.59 (s, 3H), 1.49 (s, 9H).

### Synthesis of Int-5D

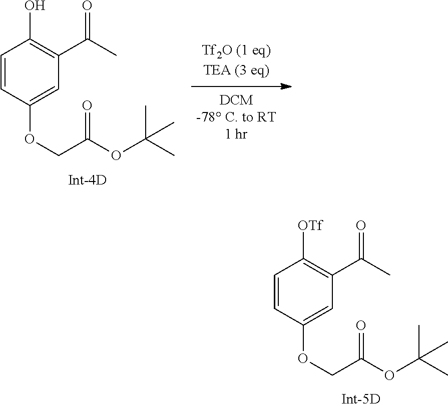 [SMILES: CC(=O)c1cc(OCC(=O)OC(C)(C)C)ccc1O ; CC(=O)c1cc(OCC(=O)OC(C)(C)C)ccc1OS(=O)(=O)C(F)(F)F]

Int-4D (250 mg, 0.94 mmol, 1 eq) was dissolved in 1 mL of dry DCM under an Ar atmosphere. Triethylamine (350 mg, 2.8 mmol, 3 eq) was added and the solution stirred at room temperature for 5 minutes. Next, the solution was cooled to −78° C. and triflic anhydride (265 mg, 0.94 mmol, 1 eq) was added dropwise. The solution was stirred for 1 hr, then quenched with 15 mL of saturated sodium bicarbonate. The mixture was extracted 3× with 10 mL of DCM, and the combined organic layers were washed once with 15 mL of brine. The combined organic layers were dried over sodium sulfate, concentrated, and the residue was purified on a silica column (0:100 to 100:0 ethyl acetate:hexanes gradient) to yield Int-5D as a clear oil (170 mg, 45.4% yield). ¹H NMR (700 MHz, CDCl₃) δ 7.21 (d, J=3.0 Hz, 1H), 7.17 (d, J=9.0 Hz, 1H), 6.98 (dd, J=9.1, 3.2 Hz, 1H), 4.49 (d, J=1.2 Hz, 2H), 2.53-2.50 (m, 3H), 1.41 (d, J=1.7 Hz, 8H).

### Synthesis of Int-6D

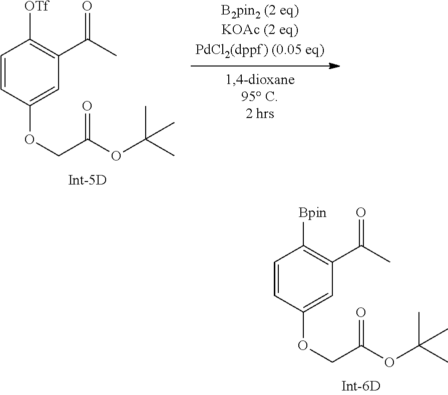

Int-5D (150 mg, 0.38 mmol, 1 eq), potassium acetate (74 mg, 0.76 mmol, 2 eq), bis(dipinacolato)diboron (191 mg, 0.76 mmol, 2 eq) were dissolved in 5 mL of dry 1,4-dioxane under an Ar atmosphere. The solution was sparged with Ar for 15 mins, followed by the addition of PdCl₂(dppf) (15.4 mg, 0.019 mmol, 0.05 eq). The solution was sparged for 15 mins again, followed by heating to 95° C. for 2 hrs. The solution was then cooled and filtered through Celite™. 15 mL of water were added, and the solution was extracted 3× with 15 mL ethyl acetate followed by washing with brine. The organic layers were combined and dried with sodium sulfate, then concentrated and purified on a silica column (0:100 to 100:0 ethyl acetate:hexanes gradient) to yield Int-6D as a brown oil (39.5 mg, 28% yield). ¹H NMR (700 MHz, CD₃CN) δ 7.62 (ddd, J=7.7, 1.5, 0.9 Hz, OH), 7.47-7.42 (m, 1H), 7.39 (d, J=2.5 Hz, OH), 7.16 (ddd, J=8.3, 2.7, 0.9 Hz, OH), 7.10 (dd, J=8.1, 2.5 Hz, OH), 4.65 (d, J=7.8 Hz, 2H), 2.57 (d, J=4.4 Hz, 3H), 1.49 (d, J=1.1 Hz, 9H), 1.37 (s, 5H), 1.27-1.20 (m, 8H).

### Synthesis of Int-7D

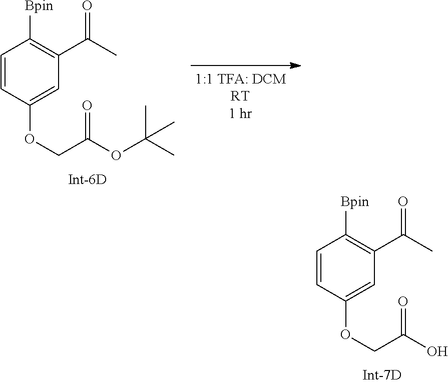

Int-6D (39.5 mg, 0.11 mmol, 1 eq) was dissolved in 2 mL of 1:1 TFA:DCM and stirred for 1 hr at room temperature. Solvent was removed in vacuo and the crude product carried through to the next step.

### Synthesis of Int-4E

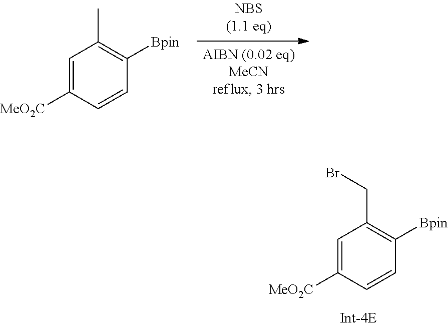

Methyl 3-methyl-4-(4,4,5,5-tetramethyl-1,3,2-dioxaborolan-2-yl)benzoate (276 mg, 1 mmol, 1 eq) was dissolved in 40 mL of acetonitrile. Next, N-bromosuccinimide (NBS) (35.6 mg, 0.2 mmol, 0.2 eq) and azobisisobutyronitrile (AIBN) (1.6 mg, 0.01 mmol, 0.01 eq) were added and the solution was heated to 70° C. Next, additional NBS (142.4 mg, 0.8 mmol, 0.8 eq) was added over the course of 1 hour, and the solution stirred for an additional 6 hours. The solvent was removed in vacuo, and the crude material purified on a silica column (0:100 to 20:80 methanol:dichloromethane gradient). Int-4E was obtained as a yellow oil (284.2 mg, 80% yield). ¹H NMR (700 MHz, CDCl₃) δ 8.03 (d, J=1.6 Hz, 1H), 7.92 (dd, J=7.7, 1.6 Hz, 1H), 7.88 (d, J=7.7 Hz, 1H), 4.92 (s, 2H), 3.92 (d, J=0.9 Hz, 3H), 1.38 (d, J=1.1 Hz, 12H).

### Synthesis of Int-5E

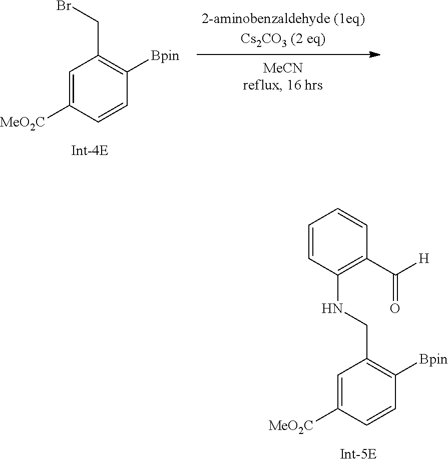

Int-4E (200 mg, 0.563 mmol, 1.1 eq) was dissolved in 1 mL of dry acetonitrile along with 2-aminobenzaldehyde (62.5 mg, 0.512 mmol, 1 eq) and cesium carbonate (166.8 mg, 0.512 mmol, 1 eq). The reaction was stirred at 60° C. for 12 hours, followed by solvent removal in vacuo. The crude material was purified on a silica column (0:100 to 100:0 ethyl acetate:hexanes gradient). Int-5E was obtained as a yellow oil (53.1 mg, 26.2% yield). ¹H NMR (700 MHz, CDCl₃) δ 9.82 (s, 1H), 8.80 (t, J=6.1 Hz, 1H), 8.03 (s, 1H), 7.95-7.89 (m, 2H), 7.45 (dd, J=7.8, 1.5 Hz, 1H), 7.33 (td, J=7.8, 1.6 Hz, 1H), 6.74 (d, J=8.6 Hz, 1H), 6.67 (t, J=7.4 Hz, 1H), 4.73 (d, J=6.1 Hz, 2H), 3.89 (d, J=1.0 Hz, 3H), 1.34 (d, J=1.0 Hz, 12H).

### Synthesis of Int-6E

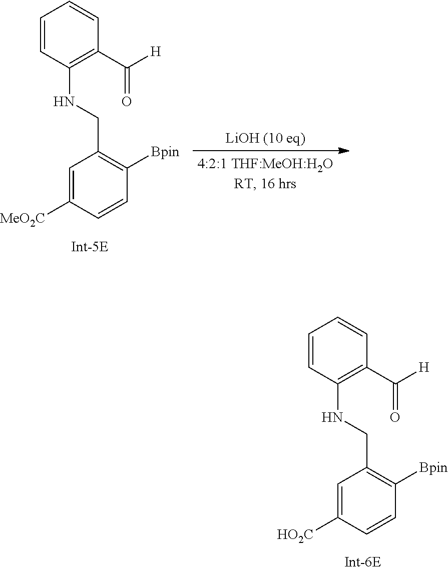

Int-5E (50 mg, 0.126 mmol, 1 eq) was dissolved in 1 mL of 4:2:1 THF:methanol:water. Next, lithium hydroxide (30.2 mg, 1.26 mmol, 10 eq) was added and the solution stirred overnight at room temperature. 5 mL of ethyl acetate and 5 mL of water were then added to the solution, and the layers separated. The aqueous layer was acidified with 1 M hydrochloric acid until a pH of 2 was reached. Then, the aqueous layer was extracted twice with 5 mL of ethyl acetate. The combined organic layers were dried over sodium sulfate and solvent removed in vacuo to yield Int-6E as an orange oil (44.3 mg, 92% yield). ¹H NMR (700 MHz, MeOD) δ 9.80 (d, J=0.7 Hz, 1H), 8.06 (dd, J=1.6, 0.7 Hz, 1H), 7.95-7.87 (m, 2H), 7.55 (ddd, J=11.8, 7.8, 1.7 Hz, 1H), 7.42-7.35 (m, 1H), 6.91-6.82 (m, 1H), 6.71 (ddd, J=7.8, 7.1, 1.0 Hz, 1H), 5.50 (s, 1H), 4.74 (s, 2H), 1.35 (s, 12H).

### Synthesis of 1A

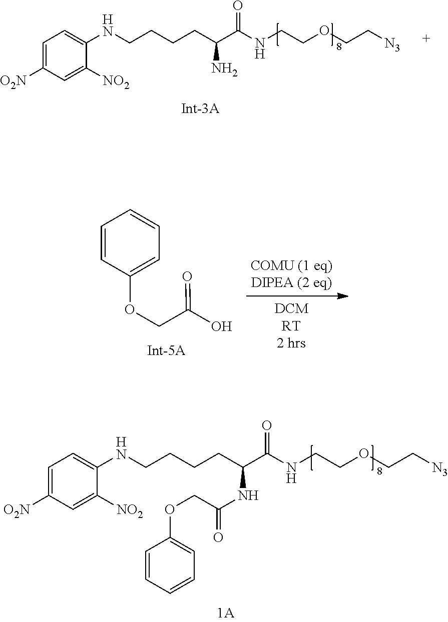

Int-3A (20 mg, 0.0273 mmol, 1 eq) was dissolved in 1 mL DCM along with Int-5A (4.15 mg, 0.0273 mmol, 1 eq) and N,N-diisopropylethylamine (7.05 mg, 0.0546 mmol, 2 eq). COMU (11.69 mg, 0.0273 mmol, 1 eq) was added and the reaction stirred for 2 hours at room temperature. Solvent was removed in vacuo and the crude mixture purified using HPLC. The resulting pure compound was dissolved in DMSO and concentrations were calculated by comparing absorbance at 360 nm to a DNP-glycine standard curve. LC-HRMS [M+H]⁺ m/z calc for [C₃₈H₅₉N₈O₁₅]: 867.4100, found 867.3795.

### Synthesis of 2A

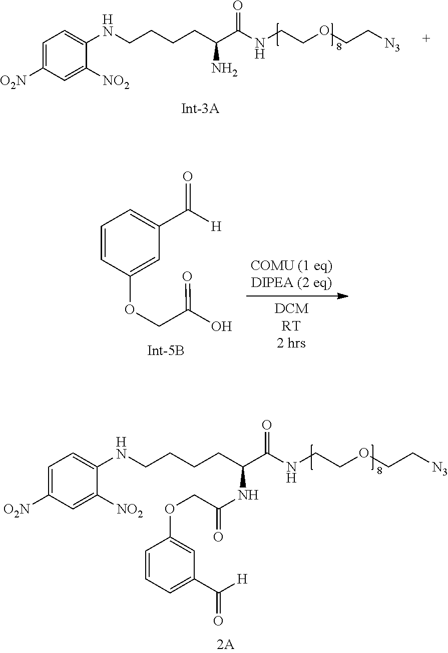

Int-3A (20 mg, 0.0273 mmol, 1 eq) was dissolved in 1 mL DCM along with Int-5B (4.91 mg, 0.0273 mmol, 1 eq) and N,N-diisopropylethylamine (7.05 mg, 0.0546 mmol, 2 eq). COMU (11.69 mg, 0.0273 mmol, 1 eq) was added and the reaction stirred for 2 hours at room temperature. Solvent was removed in vacuo and the crude mixture purified using HPLC. The resulting pure compound was dissolved in DMSO and concentrations were calculated by comparing absorbance at 360 nm to a DNP-glycine standard curve. LC-HRMS [M+H]⁺ m/z calc for [C₃₉H₅₉N₈O₁₆]: 895.4049, found 895.2935.

### Synthesis of 3A

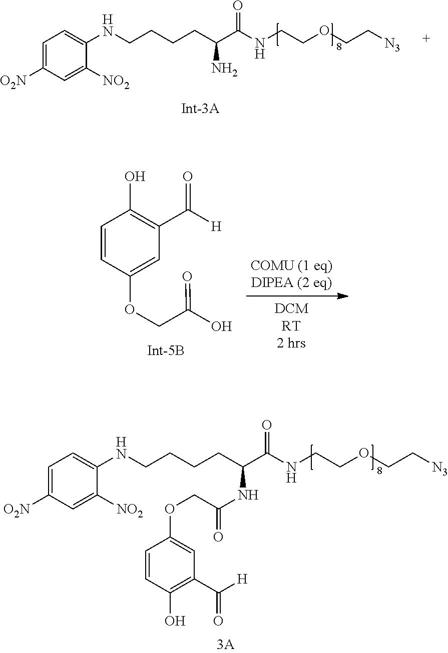

Int-3A (20 mg, 0.0273 mmol, 1 eq) was dissolved in 1 mL DCM along with Int-5C (5.36 mg, 0.0273 mmol, 1 eq) and N,N-diisopropylethylamine (7.05 mg, 0.0546 mmol, 2 eq). COMU (11.69 mg, 0.0273 mmol, 1 eq) was added and the reaction stirred for 2 hours at room temperature. Solvent was removed in vacuo and the crude mixture purified using HPLC. The resulting pure compound was dissolved in DMSO and concentrations were calculated by comparing absorbance at 360 nm to a DNP-glycine standard curve. LC-HRMS [M+H]⁺ m/z calc for [C₃₉H5₉N8O₁₇]: 911.3998, found 911.2829.

### Synthesis of 4A

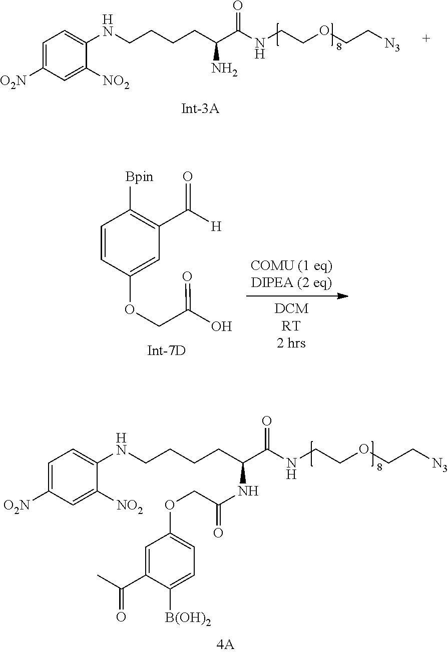

Int-3A (20 mg, 0.0273 mmol, 1 eq) was dissolved in 1 mL DCM along with Int-7D (8.74 mg, 0.0273 mmol, 1 eq) and N,N-diisopropylethylamine (7.05 mg, 0.0546 mmol, 2 eq). COMU (11.69 mg, 0.0273 mmol, 1 eq) was added and the reaction stirred for 2 hours at room temperature. Solvent was removed in vacuo and the crude mixture purified using HPLC. The resulting pure compound was dissolved in DMSO and concentrations were calculated by comparing absorbance at 360 nm to a DNP-glycine standard curve. LC-HRMS [M+H−H₂O]⁺ m/z calc for [C₄₀H₆₀BN₈O₁₇]: 935.4170, found 935.5188.

### Synthesis of 5A

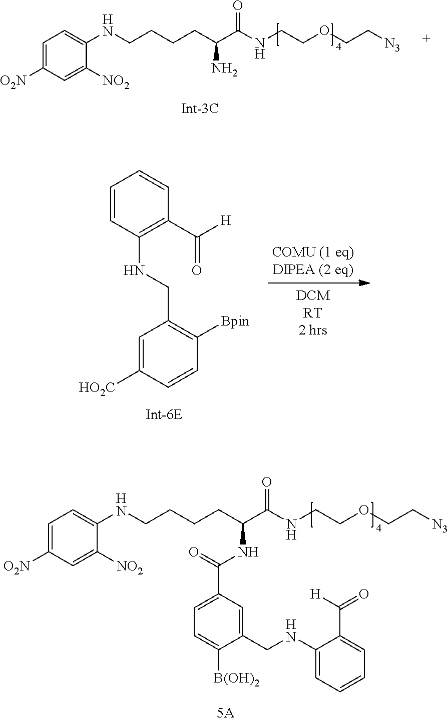

Int-3C (26 mg, 0.047 mmol, 1 eq) was dissolved in 1 mL DMF along with Int-6E (17.8 mg, 0.047 mmol, 1 eq) and N,N-diisopropylethylamine (12.3 mg, 0.094 mmol, 2 eq). COMU (20.0 mg, 0.047 mmol, 1 eq) was added and the reaction stirred for 2 hours at room temperature. Solvent was removed in vacuo and the crude mixture purified using HPLC. 5A was obtained as a yellow oil (2.1 mg, 2.7% yield)¹H NMR (700 MHz, CDCl₃) δ 9.81 (s, 1H), 9.13-9.09 (m, 1H), 8.73 (t, J=6.0 Hz, 1H), 8.53 (t, J=5.4 Hz, 1H), 8.23 (dd, J=9.5, 2.7 Hz, 1H), 7.91 (d, J=7.7 Hz, 1H), 7.82 (d, J=1.7 Hz, 1H), 7.64 (dd, J=7.7, 1.7 Hz, 1H), 7.45 (dd, J=7.7, 1.7 Hz, 1H), 7.34 (ddd, J=8.7, 7.1, 1.6 Hz, 1H), 7.00 (d, J=7.8 Hz, 1H), 6.90 (t, J=12.1 Hz, 2H), 6.76 (d, J=8.5 Hz, 1H), 6.67 (t, J=7.4 Hz, 1H), 4.73 (d, J=5.8 Hz, 2H), 4.66 (td, J=7.5, 5.6 Hz, 1H), 3.68-3.53 (m, 16H), 3.38 (dt, J=10.2, 6.1 Hz, 5H), 2.08-1.99 (m, 1H), 1.89-1.73 (m, 3H), 1.54 (h, J=7.1 Hz, 2H), 1.33 (s, 12H). LC-HRMS [M+H]⁺ m/z calc for [C₃₇H₄₉BN₉O₁₃]: 838.3543, found 838.1602.

### Synthesis of 6A

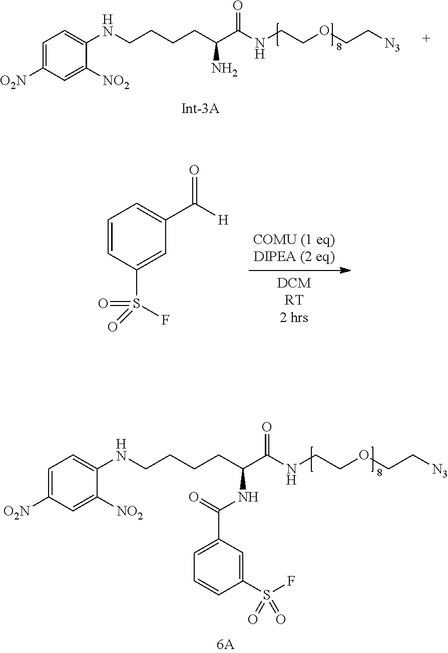

Int-3A (20 mg, 0.0273 mmol, 1 eq) was dissolved in 1 mL DCM along with 3-fluorosulfonylbenzoic acid (5.57 mg, 0.0273 mmol, 1 eq) and N,N-diisopropylethylamine (7.05 mg, 0.0546 mmol, 2 eq). COMU (11.69 mg, 0.0273 mmol, 1 eq) was added and the reaction stirred for 2 hours at room temperature. Solvent was removed in vacuo and the crude mixture purified using HPLC. The resulting pure compound was dissolved in DMSO and concentrations were calculated by comparing absorbance at 360 nm to a DNP-glycine standard curve. LC-HRMS [M+H]⁺ m/z calc for [C₃₇H₅₆FN₈O₁₆S]: 919.3519, found 919.1803.

### Synthesis of 1B

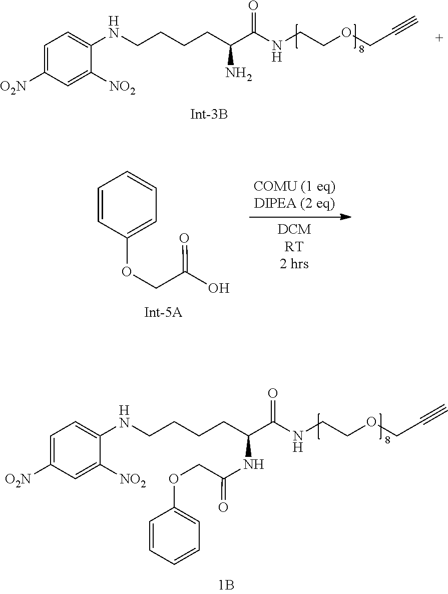

Int-3B (12.6 mg, 0.018 mmol, 1 eq) was dissolved along with Int-5A (2.74 mg, 0.018 mmol, 1 eq) and N,N-diisopropylethylamine (5 mg, 0.036 mmol, 2 eq) in 1 mL DCM. Next, COMU (7.5 mg, 0.018 mmol, 1 eq) was added and the reaction stirred for 2 hours at room temperature. Solvent was removed in vacuo and the crude mixture purified using HPLC. The resulting pure compound was dissolved in DMSO and concentrations were calculated by comparing absorbance at 360 nm to a DNP-glycine standard curve. 1B was obtained as a yellow oil (9.3 mg, 62% yield). ¹H NMR (700 MHz, CDCl₃) δ 9.06 (d, J=2.7 Hz, 1H), 8.45 (t, J=5.2 Hz, 1H), 8.20 (ddd, J=9.4, 2.6, 0.7 Hz, 1H), 7.28-7.22 (m, 2H), 7.19 (s, 2H), 6.95 (tt, J=7.4, 1.0 Hz, 1H), 6.90-6.85 (m, 2H), 6.84 (d, J=9.5 Hz, 1H), 6.72 (s, 1H), 4.50-4.41 (m, 3H), 4.13 (d, J=2.4 Hz, 2H), 3.65-3.53 (m, 31H), 3.51-3.46 (m, 2H), 3.39 (qd, J=6.0, 4.0 Hz, 2H), 3.30 (td, J=7.2, 5.3 Hz, 2H), 2.37 (t, J=2.4 Hz, 1H), 1.90 (dtd, J=13.8, 7.7, 5.6 Hz, 1H), 1.79-1.64 (m, 3H), 1.44-1.35 (m, 2H). LC-HRMS [M+H₂O]⁺ m/z calc for [C₃₉H₅₉N₅O₁₆]: 853.3957, found 853.2028.

### Synthesis of 2B

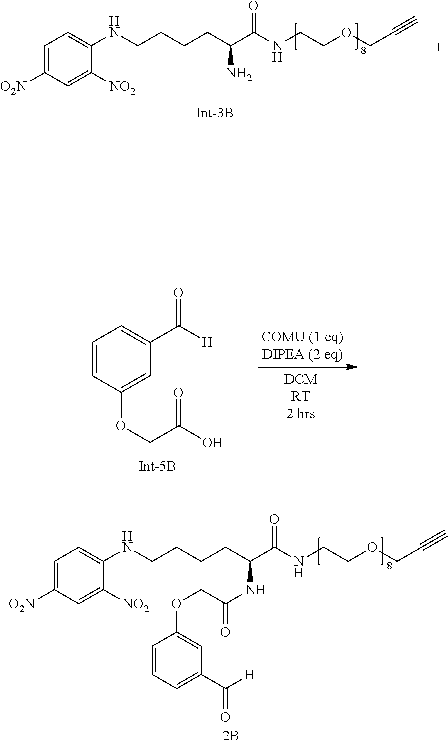

Int-3B (12.6 mg, 0.018 mmol, 1 eq) was dissolved along with Int-5B (3.24 mg, 0.018 mmol, 1 eq) and N,N-diisopropylethylamine (5 mg, 0.036 mmol, 2 eq) in 1 mL DCM. Next, COMU (7.5 mg, 0.018 mmol, 1 eq) was added and the reaction stirred for 2 hours at room temperature. Solvent was removed in vacuo and the crude mixture purified using HPLC. The resulting pure compound was dissolved in DMSO and concentrations were calculated by comparing absorbance at 360 nm to a DNP-glycine standard curve. 2B was obtained as a yellow oil (6.5 mg, 41.8% yield). ¹H NMR (700 MHz, CDCl₃) δ 9.91 (s, 1H), 9.05 (d, J=2.6 Hz, 1H), 8.45 (t, J=5.3 Hz, 1H), 8.20 (dd, J=9.5, 2.7 Hz, 1H), 7.52-7.40 (m, 2H), 7.37-7.34 (m, 1H), 7.22-7.16 (m, 4H), 6.85 (d, J=9.5 Hz, 1H), 6.74 (s, 1H), 4.49 (dd, J=15.0, 5.2 Hz, 3H), 4.12 (d, J=2.4 Hz, 2H), 3.72-3.46 (m, 33H), 3.43-3.35 (m, 2H), 3.32 (td, J=7.1, 5.2 Hz, 2H), 2.36 (t, J=2.5 Hz, 1H), 1.89 (dtd, J=13.7, 8.0, 5.6 Hz, 1H), 1.81-1.69 (m, 1H), 1.41 (p, J=7.8 Hz, 2H). LC-HRMS [M+H]⁺ m/z calc for [C₄₀H₅₈N₅O₁₆]: 864.3879, found 864.1962.

### Synthesis of 3B

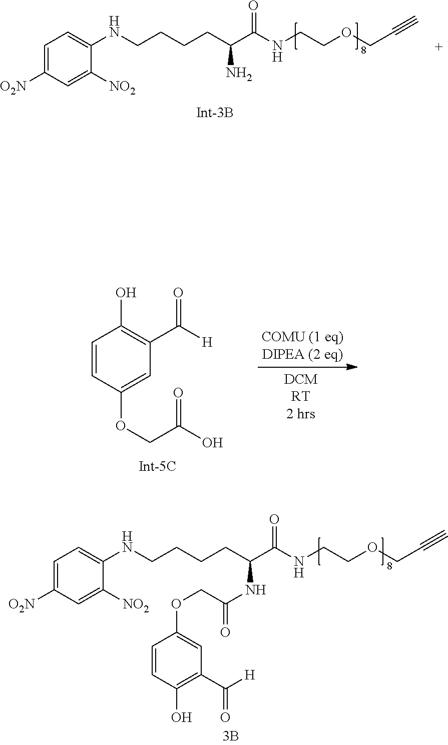

Int-3B (9.8 mg, 0.05 mmol, 1 eq) was dissolved along with Int-5C (35.1 mg, 0.05 mmol, 1 eq) and N,N-diisopropylethylamine (12.7 mg, 0.1 mmol, 2 eq) in 1 mL DCM. Next, COMU (21.4 mg, 0.05 mmol, 1 eq) was added and the reaction stirred for 2 hours at room temperature. Solvent was removed in vacuo and the crude mixture purified using HPLC. The resulting pure compound was dissolved in DMSO and concentrations were calculated by comparing absorbance at 360 nm to a DNP-glycine standard curve. 3B was obtained as a yellow oil (7.5 mg, 16.7% yield). ¹H NMR (700 MHz, CDCl₃) δ 9.93 (s, 1H), 9.07 (d, J=2.7 Hz, 1H), 8.56 (t, J=5.3 Hz, 1H), 8.25 (dd, J=9.5, 2.7 Hz, 1H), 8.19 (d, J=8.0 Hz, 1H), 7.18 (d, J=3.1 Hz, 1H), 7.05 (dd, J=8.8, 3.1 Hz, 1H), 6.93 (d, J=9.6 Hz, 2H), 6.72 (d, J=8.8 Hz, 1H), 4.57-4.51 (m, 2H), 4.47 (d, J=14.5 Hz, 1H), 4.18 (d, J=2.4 Hz, 2H), 3.76-3.36 (m, 36H), 2.43 (t, J=2.4 Hz, 1H), 2.07 (ddt, J=14.9, 10.4, 5.6 Hz, 1H), 1.95 (dtd, J=14.1, 9.1, 5.2 Hz, 1H), 1.91-1.80 (m, 2H), 1.63 (dhept, J=13.0, 7.4 Hz, 2H). LC-HRMS [M+H₂O]m/z calc for [C₄₀H5₉N5O₁₈]: 897.3855, found 897.1521.

### Synthesis of 4B

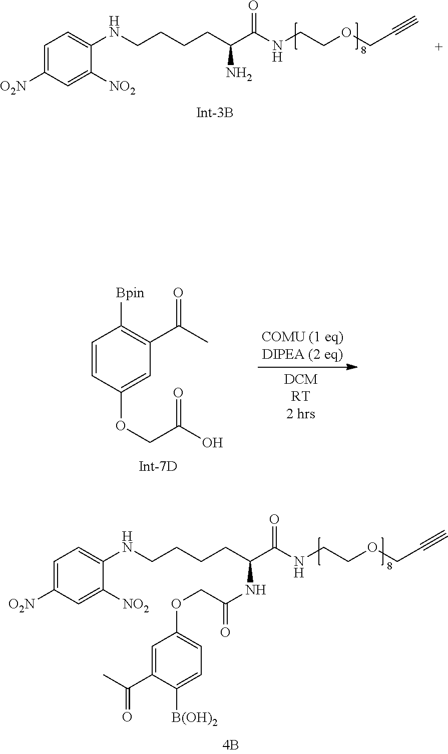

Int-3B (28.77 mg, 0.041 mmol, 1 eq) was dissolved along with Int-7D (13 mg, 0.041 mmol, 1 eq) and N,N-diisopropylethylamine (11 mg, 0.082 mmol, 2 eq) in 1 mL DCM. Next, COMU (17.6 mg, 0.041 mmol, 1 eq) was added and the reaction stirred for 2 hours at room temperature. Solvent was removed in vacuo and the crude mixture purified using HPLC. The resulting pure compound was dissolved in DMSO and concentrations were calculated by comparing absorbance at 360 nm to a DNP-glycine standard curve. LC-HRMS [M+H−H₂O]⁺ m/z calc for [C₄₁H₅₉BN₅O₁₇]: 904.3999, found 904.5178.

### Synthesis of 1C

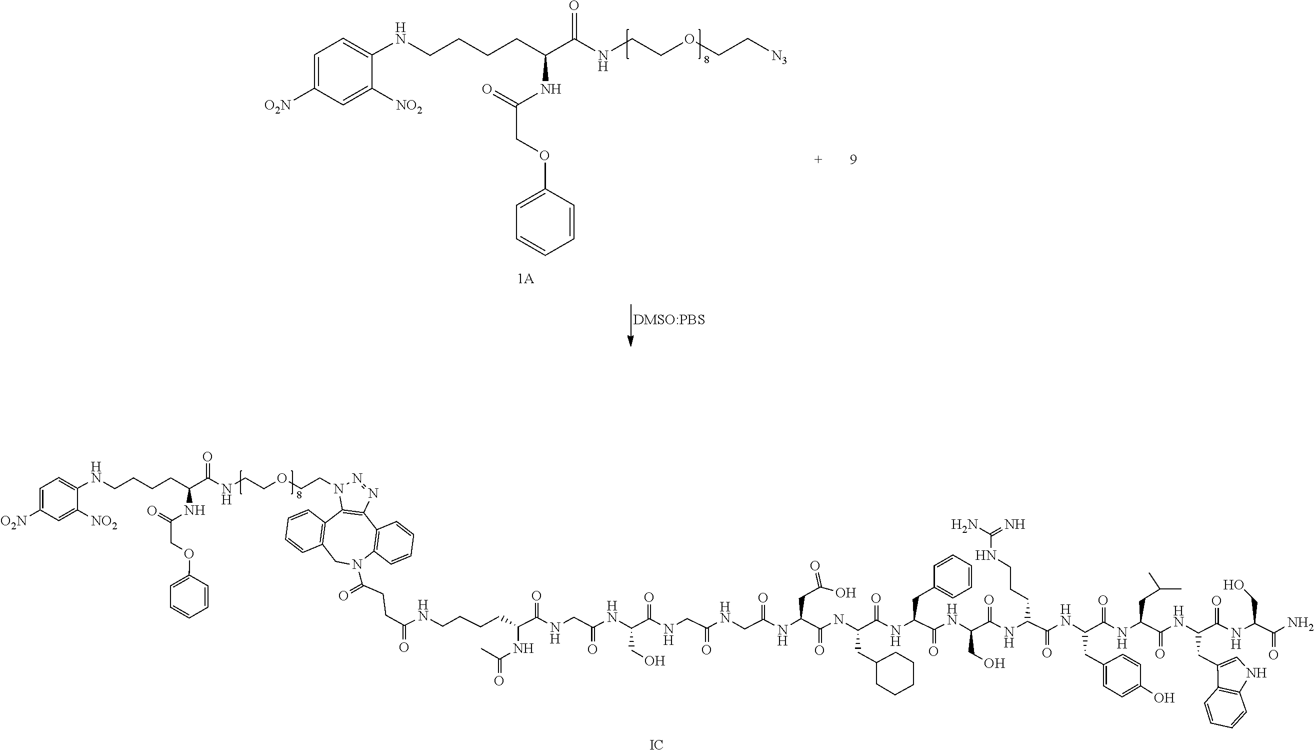

1A and 9 were combined 1:1 at a final concentration of 100 μM in 200 μL 10:90 DMSO:phosphate buffered saline (PBS). The reaction was incubated for 3 hours at room temperature, and the resulting product was aliquoted and frozen for use in assays. LC-HRMS [M+2H]²⁺ m/z calc for [C₁₃₄H₁₈₅N₂₉O₃₈]: 1404.1718, found 1404.7813.

### Synthesis of Compound I-1

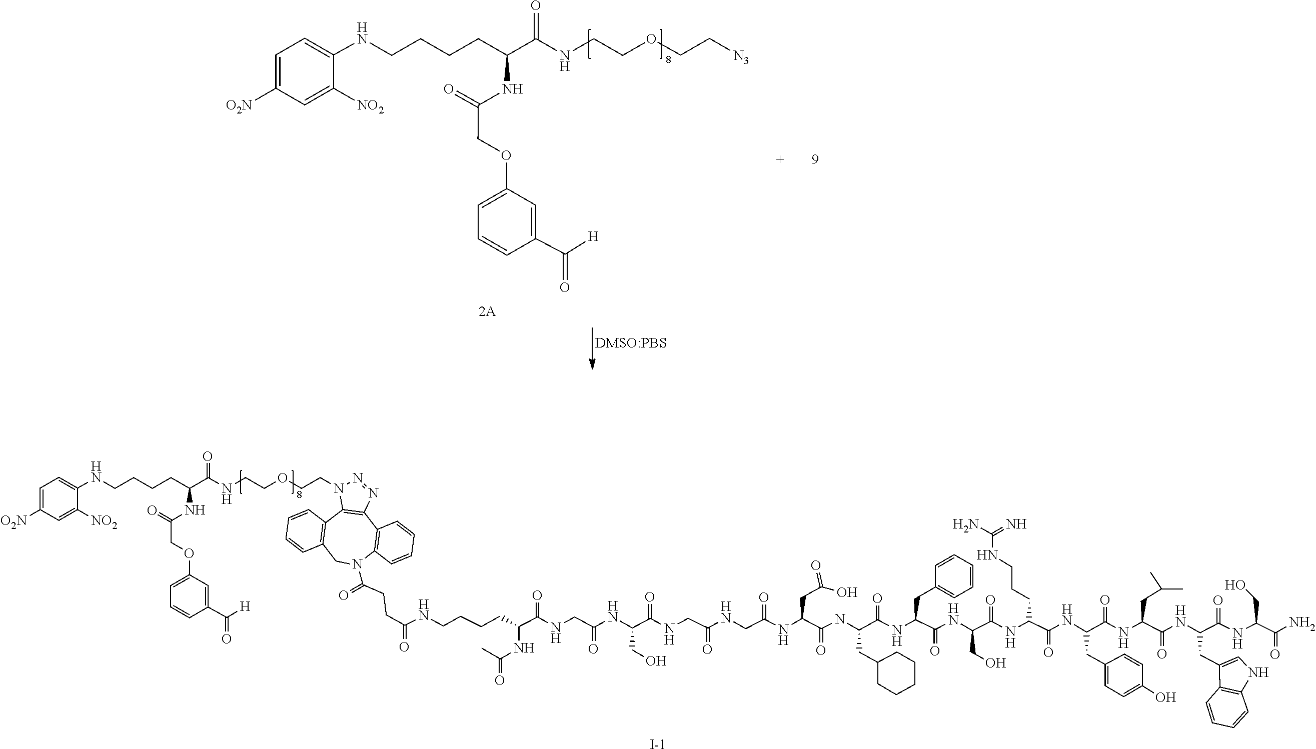

2A and 9 were combined 1:1 at a final concentration of 100 μM in 200 μL 10:90 DMSO:PBS. The reaction was incubated for 3 hours at room temperature, and the resulting product was aliquoted and frozen for use in assays. LC-HRMS [M+2H]²⁺ m/z calc for [C₁₃₅H₁₈₅N₂₉O₃₉]: 1418.1693, found 1418.1576.

### Synthesis of Compound I-2

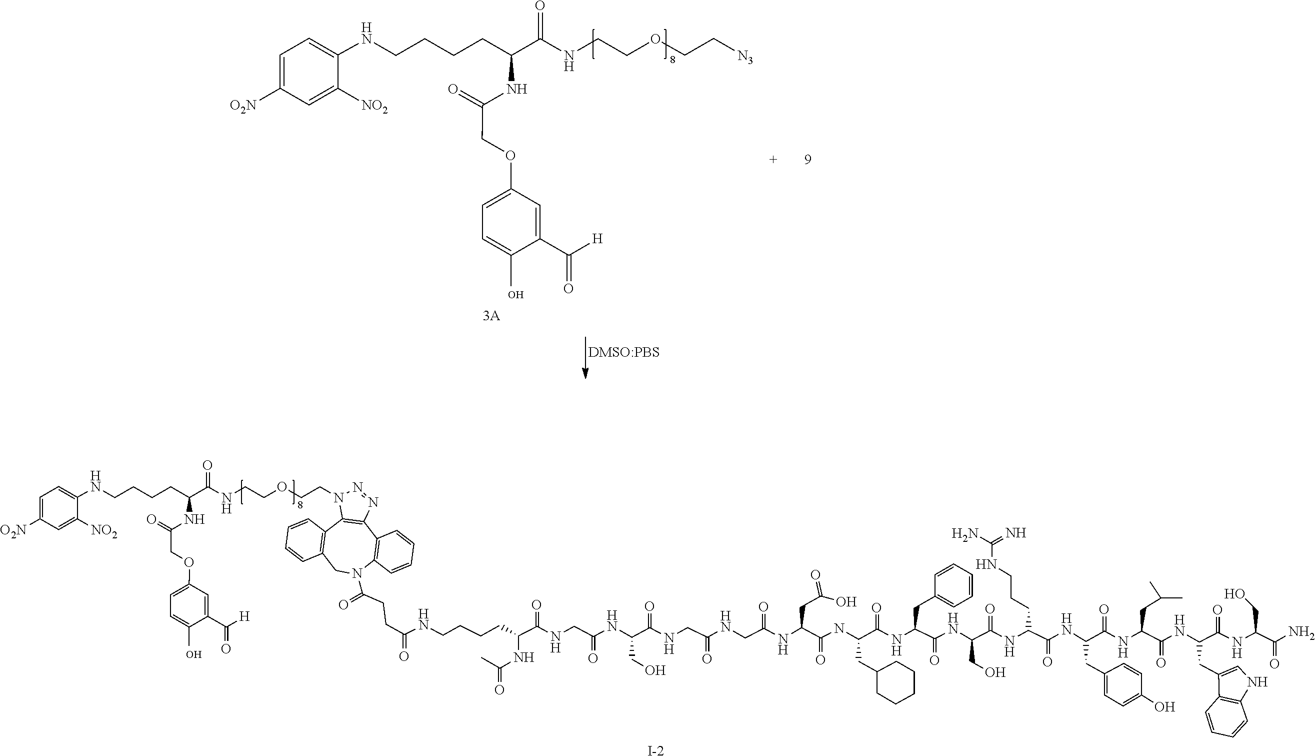

3A and 9 were combined 1:1 at a final concentration of 100 μM in 200 μL 10:90 DMSO:PBS. The reaction was incubated for 3 hours at room temperature, and the resulting product was aliquoted and frozen for use in assays. LC-HRMS [M+2H]²⁺ m/z calc for [C₁₃₅H₁₈₅N₂₉O₄₀]: 1426.1667, found 1426.1087.

### Synthesis of Compound I-3

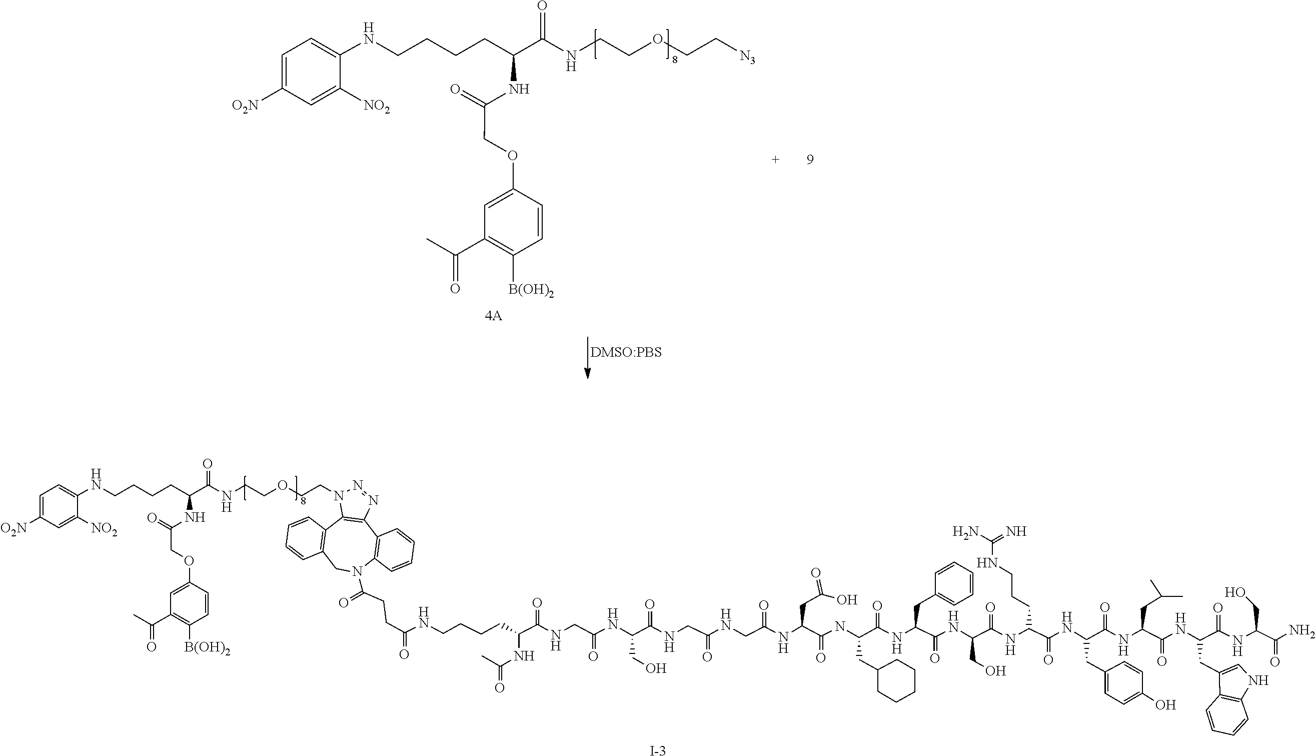

4A and 9 were combined 1:1 at a final concentration of 100 μM in 200 μL 10:90 DMSO:PBS. The reaction was incubated for 3 hours at room temperature, and the resulting product was aliquoted and frozen for use in assays. LC-HRMS [M+2H−OH]²⁺ m/z calc for [C₁₃₆H₁₈₇BN₂₉O₄₀]: 1438.6792, found 1439.0776.

### Synthesis of Compound I-4

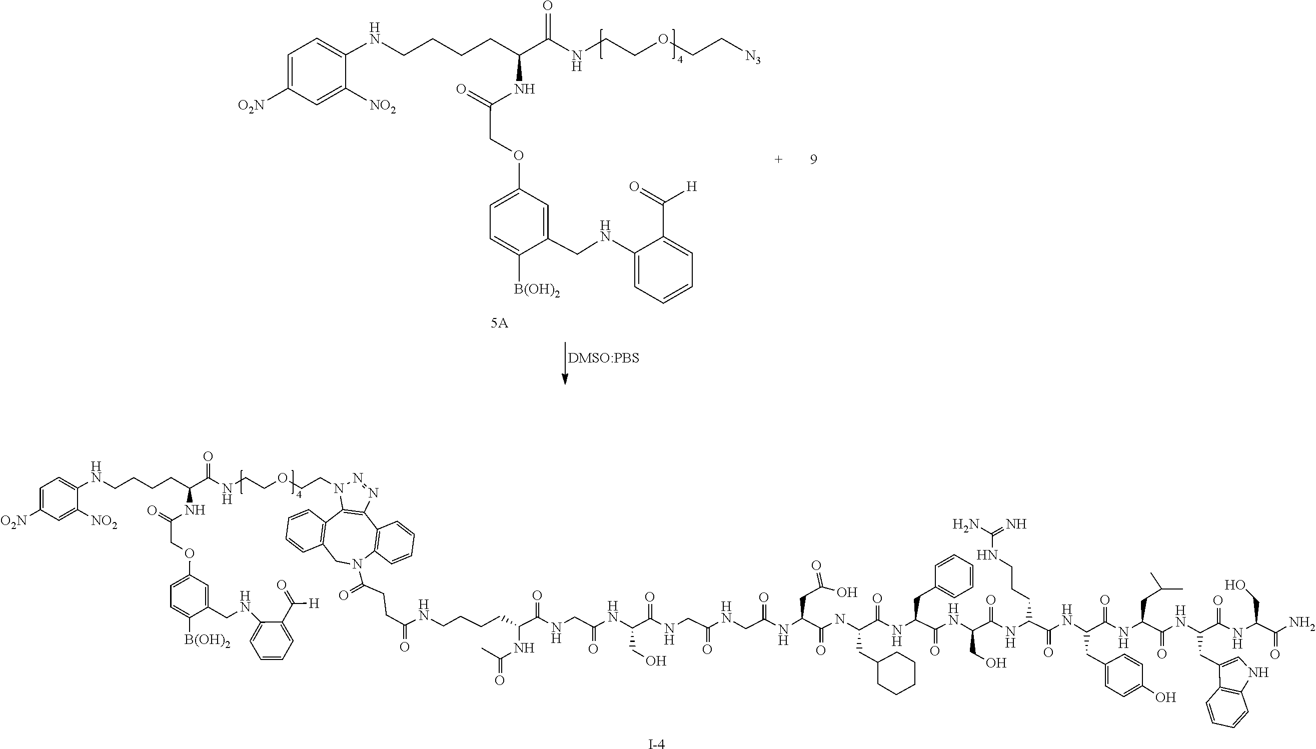

5A and 9 were combined 1:1 at a final concentration of 100 μM in 200 μL 10:90 DMSO:PBS. The reaction was incubated for 3 hours at room temperature, and the resulting product was aliquoted and frozen for use in assays. LC-HRMS [M+2H−2H₂O]²⁺ m/z calc for [C₁₃₃H₁₇₃BN₃₀O₃₄]: 1371.6334, found 1371.7263.

### Synthesis of 6C

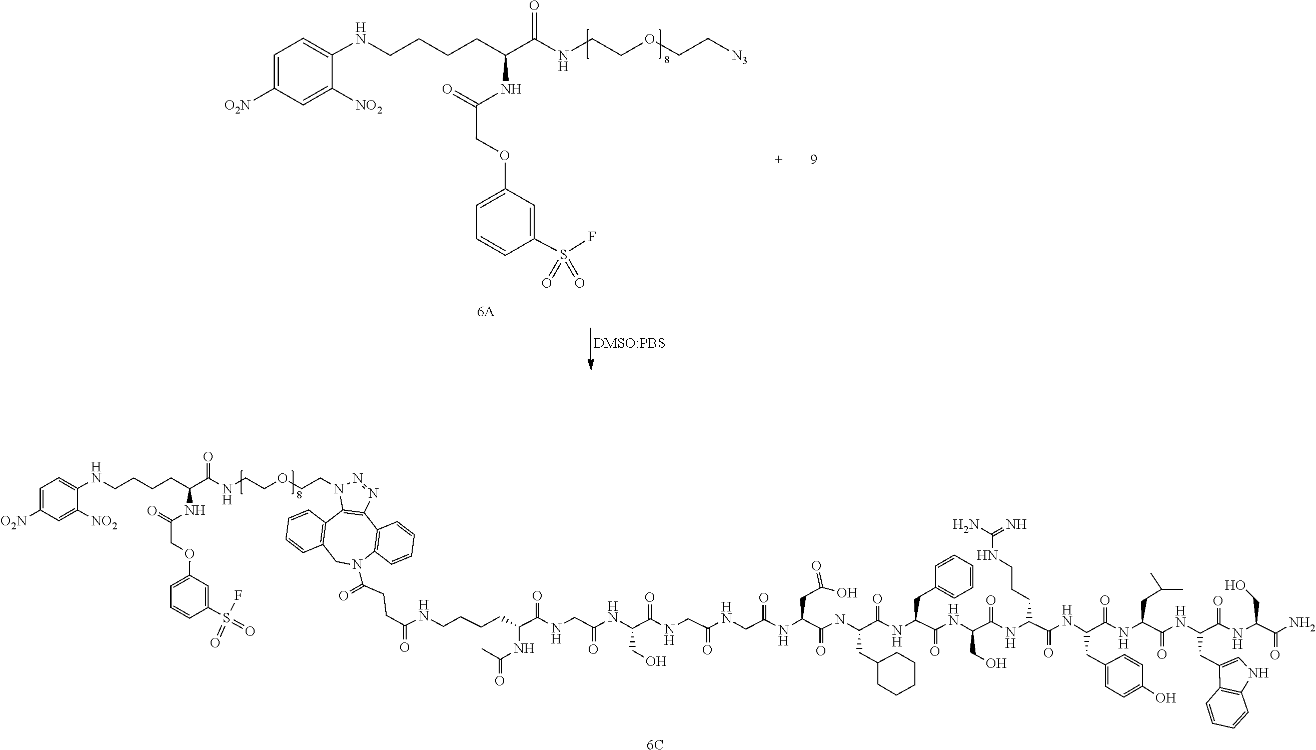

6A and 9 were combined 1:1 at a final concentration of 100 μM in 200 μL 10:90 DMSO:PBS. The reaction was incubated for 3 hours at room temperature, and the resulting product was aliquoted and frozen for use in assays. LC-HRMS [M+2H]²⁺ m/z calc for [C₁₃₃H₁₈₂FN₂₉O₃₉S]: 1430.1427, found 1430.5858.

### Synthesis of 1D

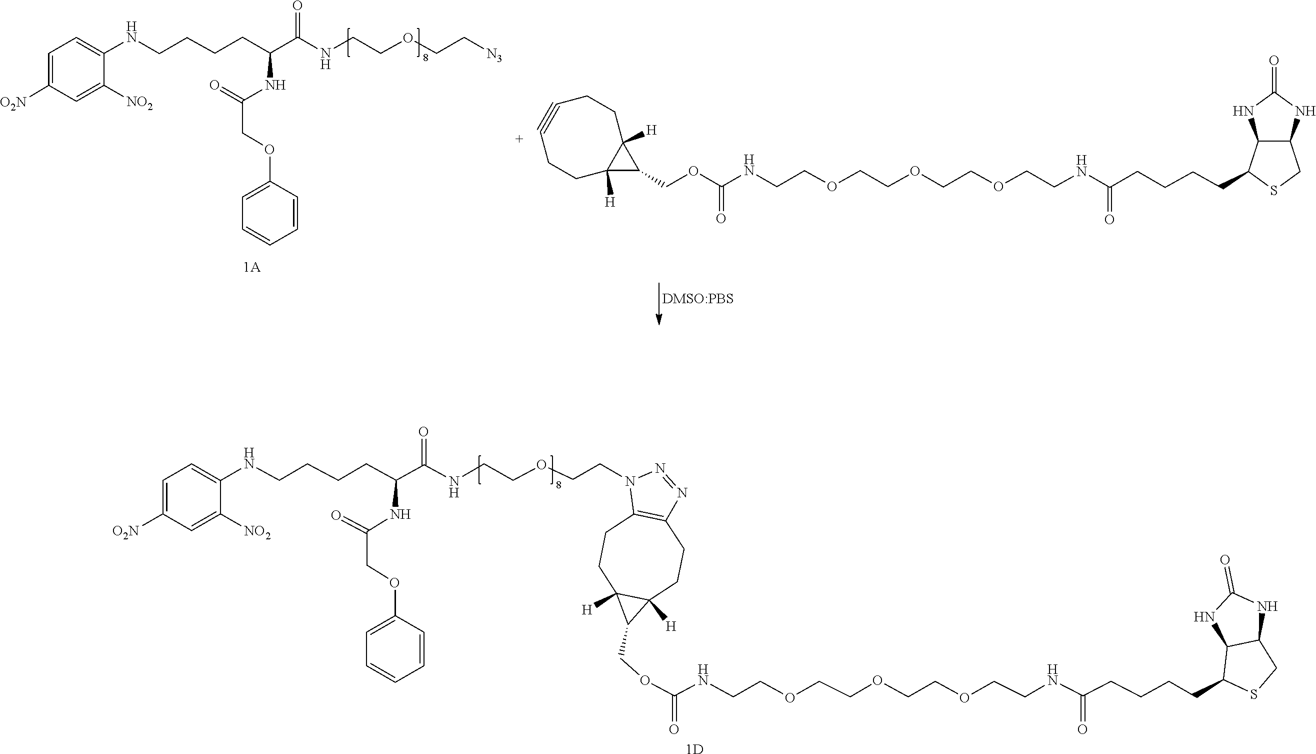

1A and BCN-endo-PEG₃-biotin were combined 1:1 at a final concentration of 200 μM in 200 μL of 10:90 DMSO:PBS. The reaction was incubated for 2 hours at room temperature, and the resulting product was aliquoted and frozen for use in assays. LC-HRMS [M+H]⁺ m/z calc for [C₆₇H₁₀₇N₁₂O₂₂S]: 1461.7187, found 1461.6326.

### Synthesis of Compound 1-5

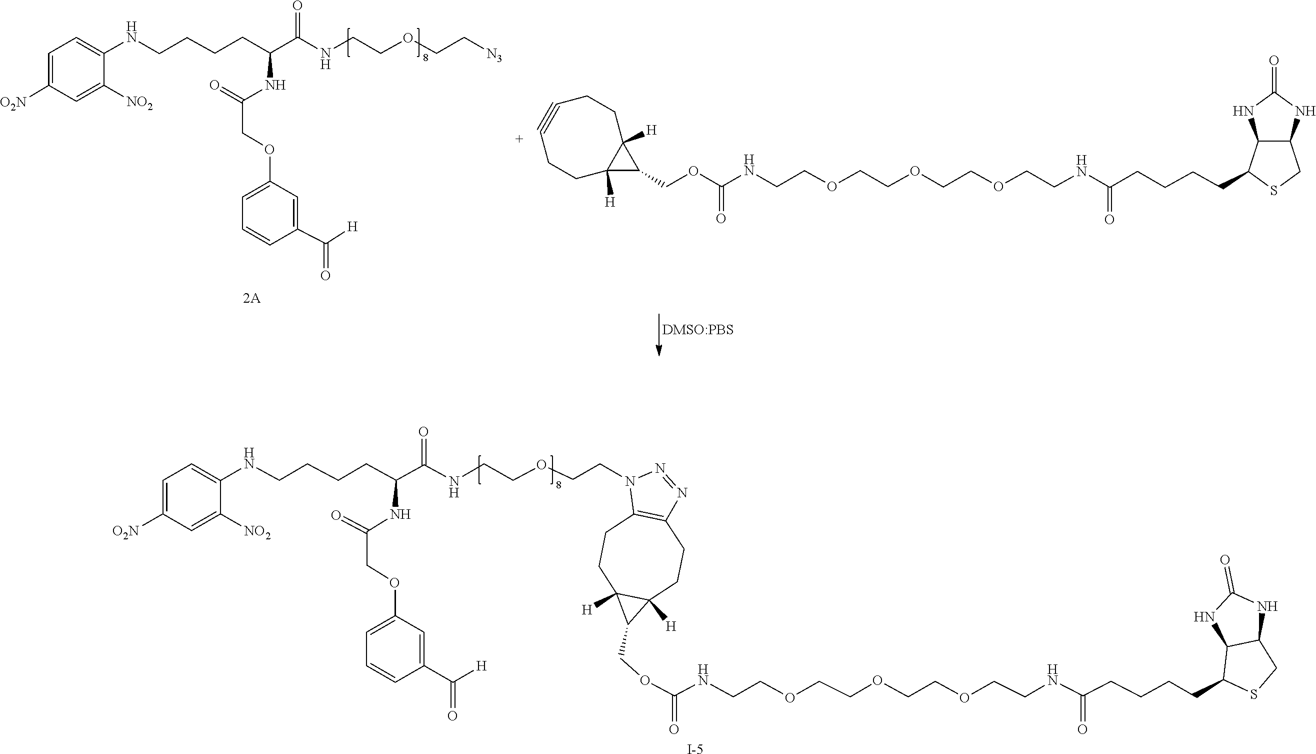

2A and BCN-endo-PEG₃-biotin were combined 1:1 at a final concentration of 200 μM in 200 μL of 10:90 DMSO:PBS. The reaction was incubated for 2 hours at room temperature, and the resulting product was aliquoted and frozen for use in assays. LC-HRMS [M+H]⁺ m/z calc for [C₆₈H₁₀₅N₁₂O₂₃S]: 1489.7136, found 1489.5767.

### Synthesis of Compound I-6

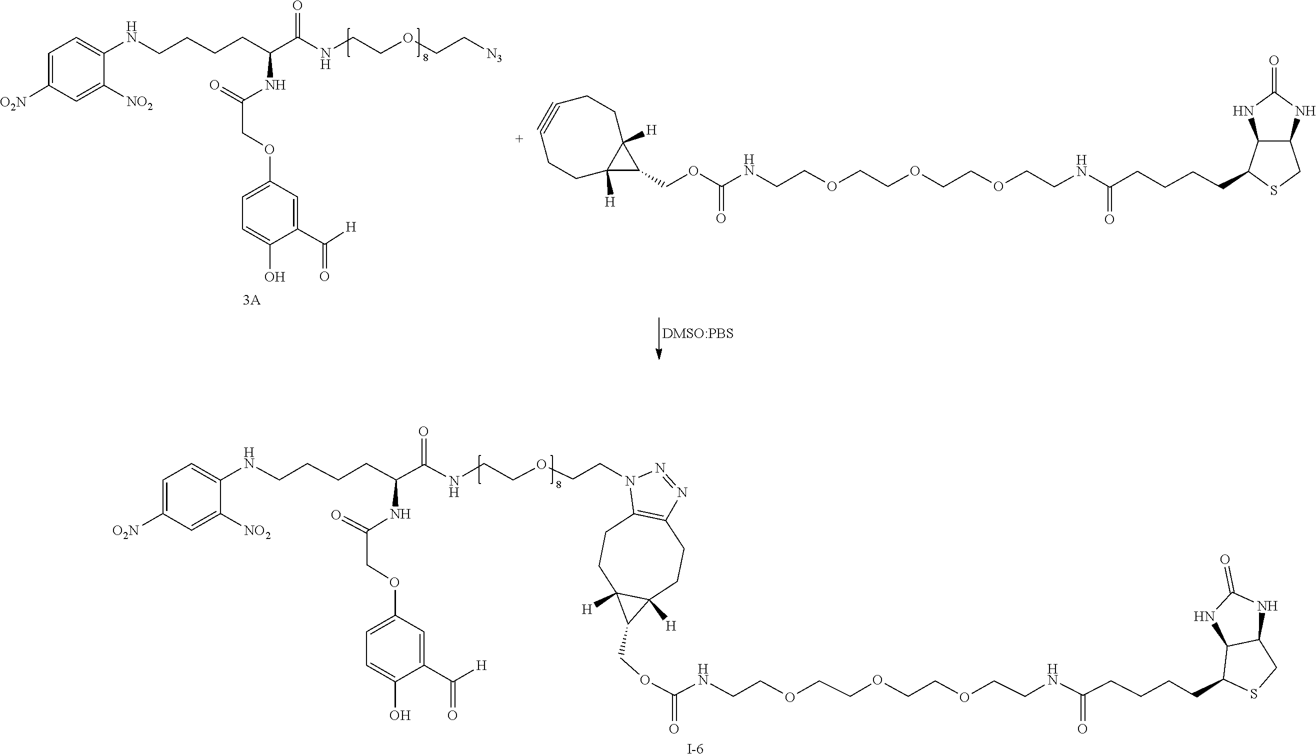

3A and BCN-endo-PEG₃-biotin were combined 1:1 at a final concentration of 200 μM in 200 μL of 10:90 DMSO:PBS. The reaction was incubated for 2 hours at room temperature, and the resulting product was aliquoted and frozen for use in assays. LC-HRMS [M+H]⁺ m/z calc for [C₆₈H₁₀₅N₁₂O₂₄S]: 1505.7085, found 1505.6038.

### Synthesis of Compound I-7

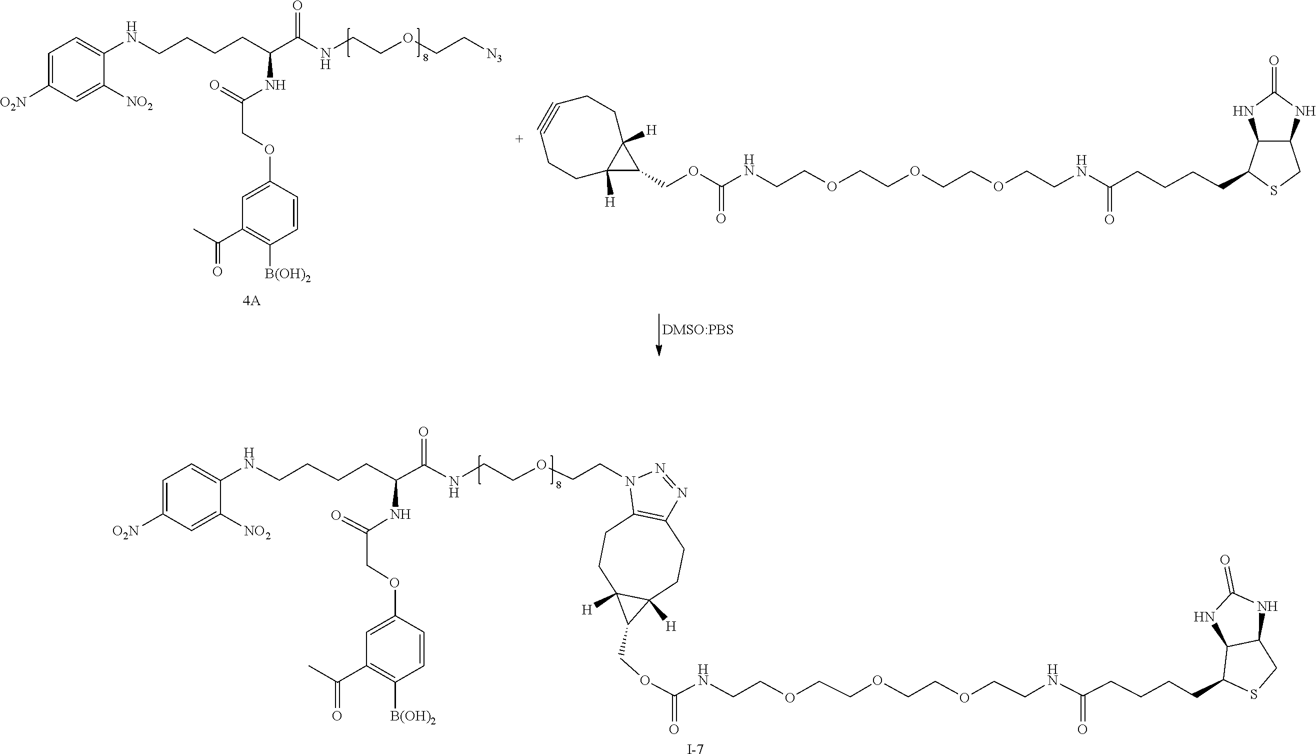

4A and BCN-endo-PEG₃-biotin were combined 1:1 at a final concentration of 200 μM in 200 μL of 10:90 DMSO:PBS. The reaction was incubated for 2 hours at room temperature, and the resulting product was aliquoted and frozen for use in assays. LC-HRMS [M+H]⁺ m/z calc for [C₆₉H₁₀₈BN₁₂O₂₅S]: 1547.7362, found 1547.6521.

### Synthesis of Compound I-8

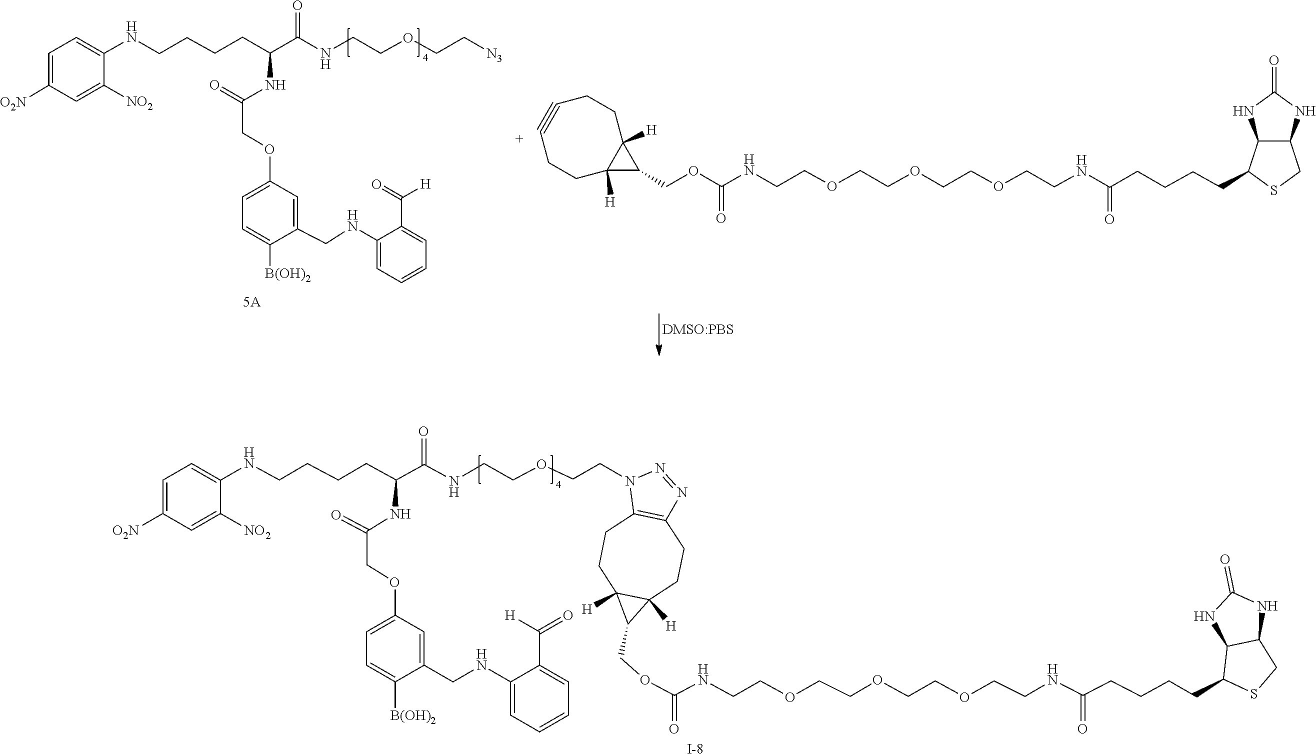

5A and BCN-endo-PEG₃-biotin were combined 1:1 at a final concentration of 200 μM in 200 μL of 10:90 DMSO:PBS. The reaction was incubated for 2 hours at room temperature, and the resulting product was aliquoted and frozen for use in assays. LC-HRMS [M+H]⁺ m/z calc for [C₆₆H₉₅BN₁₃O₂₀S]: 1432.6630, found 1432.1200.

### Synthesis of 6D

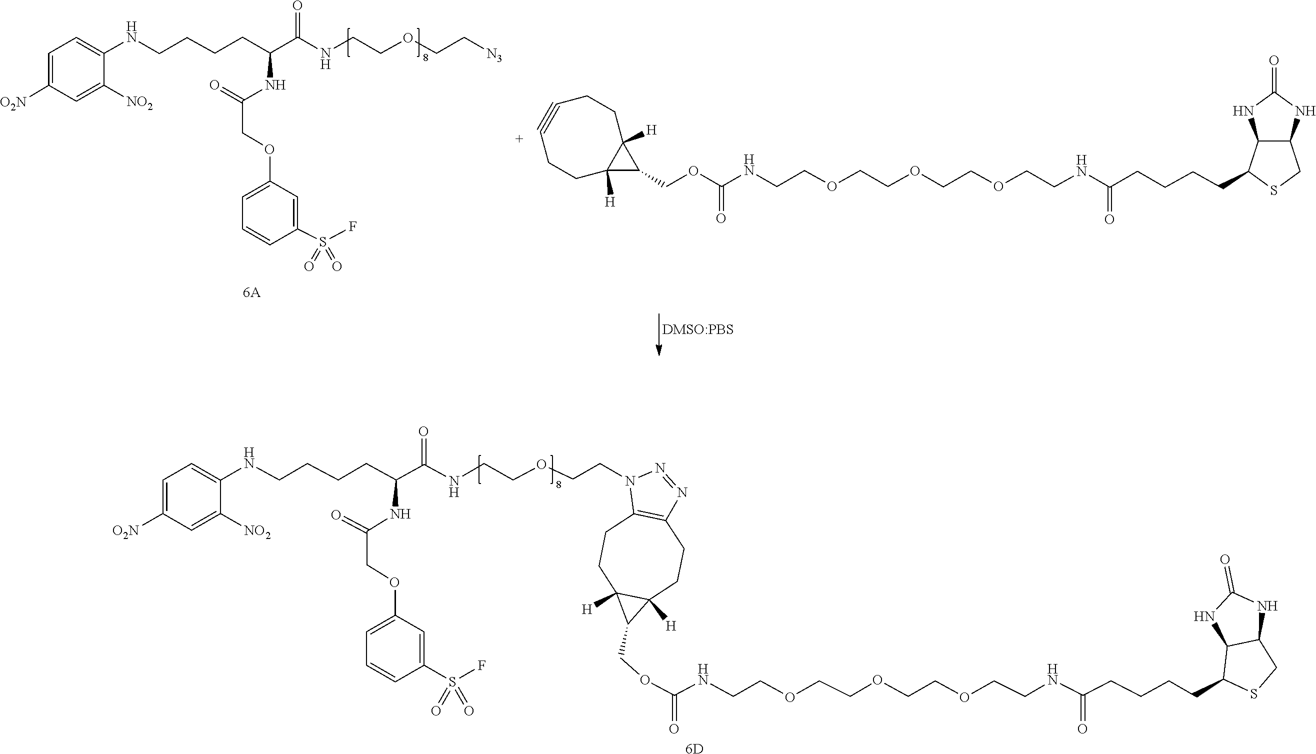

6A and BCN-endo-PEG₃-biotin were combined 1:1 at a final concentration of 200 μM in 200 μL of 10:90 DMSO:PBS. The reaction was incubated for 2 hours at room temperature, and the resulting product was aliquoted and frozen for use in assays. LC-HRMS [M+H]⁺ m/z calc for [C₆₆H₁₀₂FN₁₂O₂₃S₂]: 1513.6606, found 1513.2682.

### Synthesis of Int-9

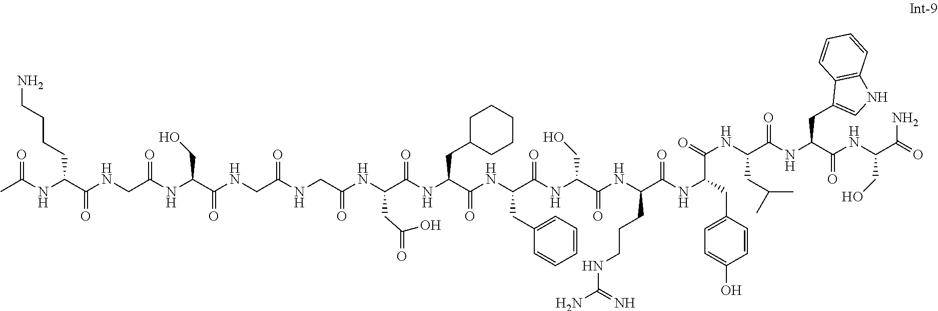 [SMILES: CC(=O)N[C@H](CCCCN)C(=O)NCC(=O)N[C@@H](CO)C(=O)NCC(=O)NCC(=O)N[C@@H](CC(=O)O)C(=O)N[C@@H](CC1CCCCC1)C(=O)N[C@@H](Cc1ccccc1)C(=O)N[C@H](CO)C(=O)N[C@H](CCCNC(=N)N)C(=O)N[C@@H](Cc1ccc(O)cc1)C(=O)N[C@@H](CC(C)C)C(=O)N[C@@H](Cc1c[nH]c2ccccc12)C(=O)N[C@@H](CO)C(N)=O]

Int-9 was synthesized on 0.1 mmol scale using Rink amide resin on a CEM Liberty Blue automated peptide synthesizer. Cleavage was performed at room temperature for 3 hours using 95:2.5:2.5 TFA:TIS:water. The cleaved peptide was precipitated using ice cold diethyl ether, followed by drying and resuspension in DMSO for HPLC purification. The peptide was obtained as a white solid and carried through to the next step. LC-MS [M+H]⁺ m/z calc for [C₇₇H₁₁₃N₂₀O₂₁]: 1653.8389, found 1654.5657.

### Synthesis of 9

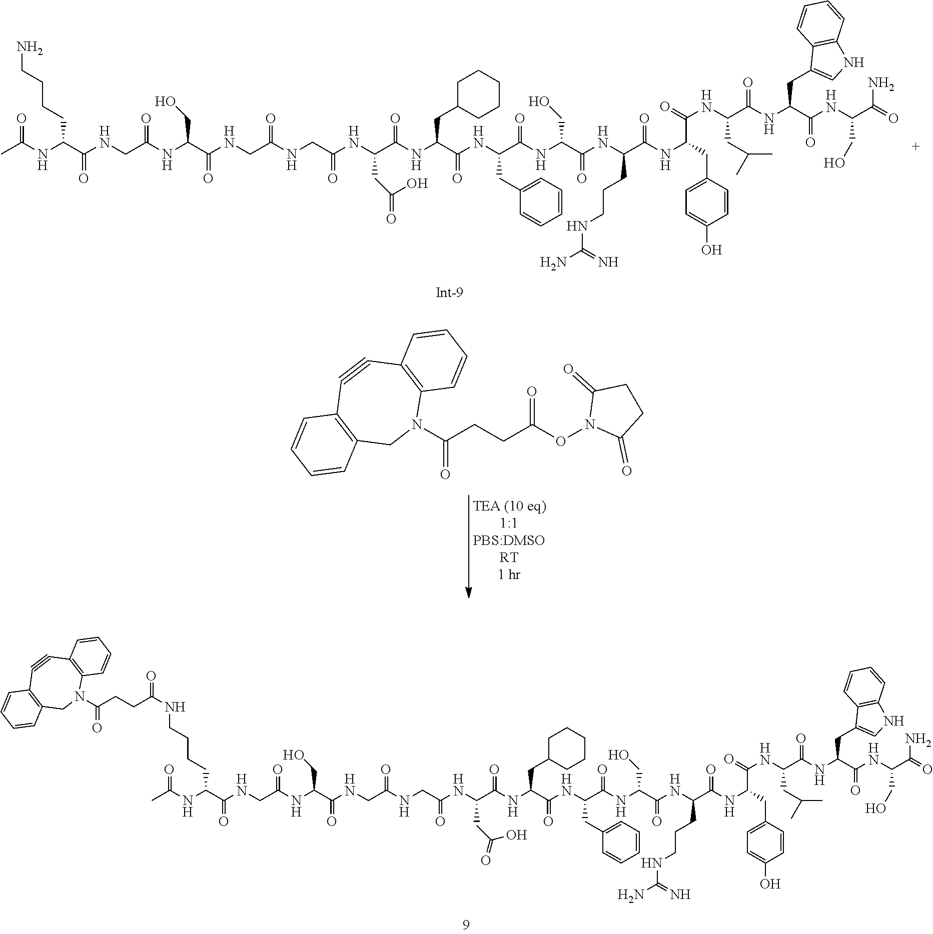

Int-9 (11.5 mg, 0.007 mmol, 1 eq) was dissolved in 1:1 DMSO:PBS along with DBCO-NHS (28 mg, 0.070 mmol, 10 eq) and TEA (0.9 mg, 0.070 mmol, 10 eq). The reaction was stirred at room temperature for 2 hours, followed by HPLC purification. The resulting white solid was resuspended in DMSO and concentration was assessed using absorbance at 310 nm compared to a DBCO-NHS standard curve. LC-HRMS [M+2H]²⁺ m/z calc for [C₉₆H₁₂₇N₂₁O₂₃]: 970.9707, found 971.3583.

### Synthesis of 1F (Ph-GUL)

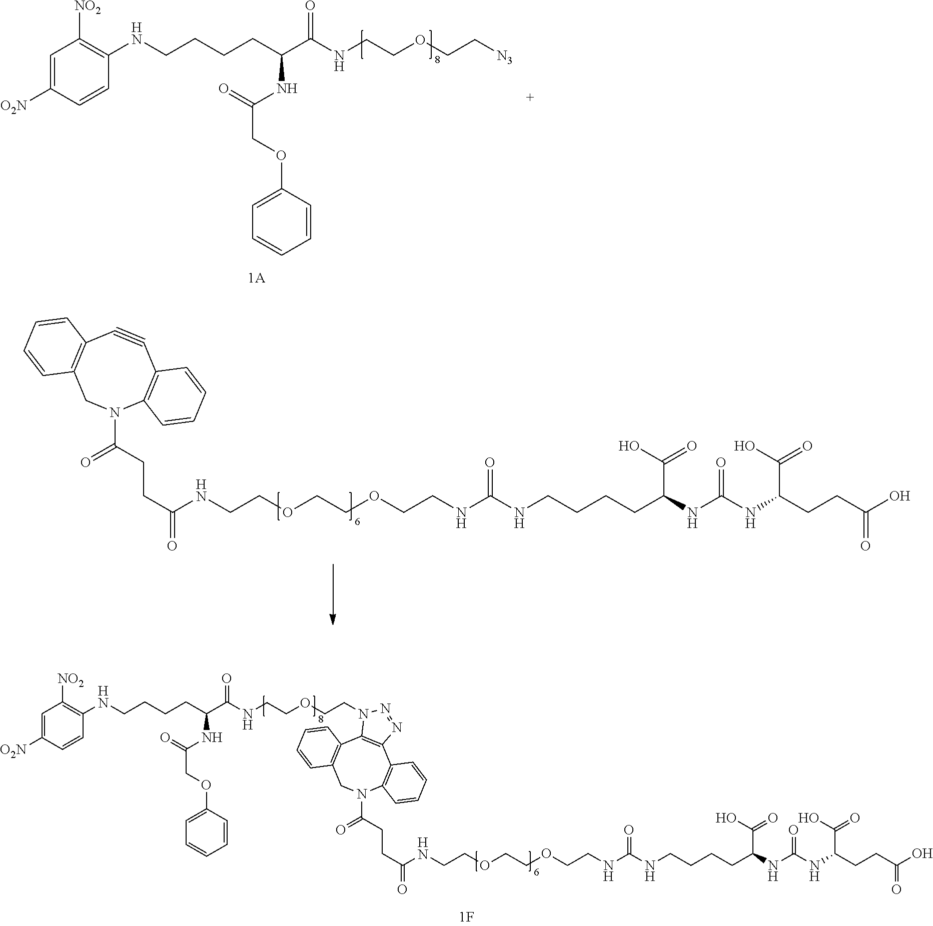

1A was combined 1:1 with DBCO-OEG₇-GUL in 1000 DMSO:PBS at a final concentration of 100 μM and a final volume of 100 μL. The reaction was heated to 37° C. for 2 hrs. 1F was used without further purification. Liquid Chromatography-Mass Spectrometry (LCMS) (ESI+) [M+2H]²⁺ m/z calc for [C₈₆H₁₂₈N₁₄O₃₂]: 934.44, found 934.76.

### Synthesis of 4F (2-APBA-GUL) (compound I-9)

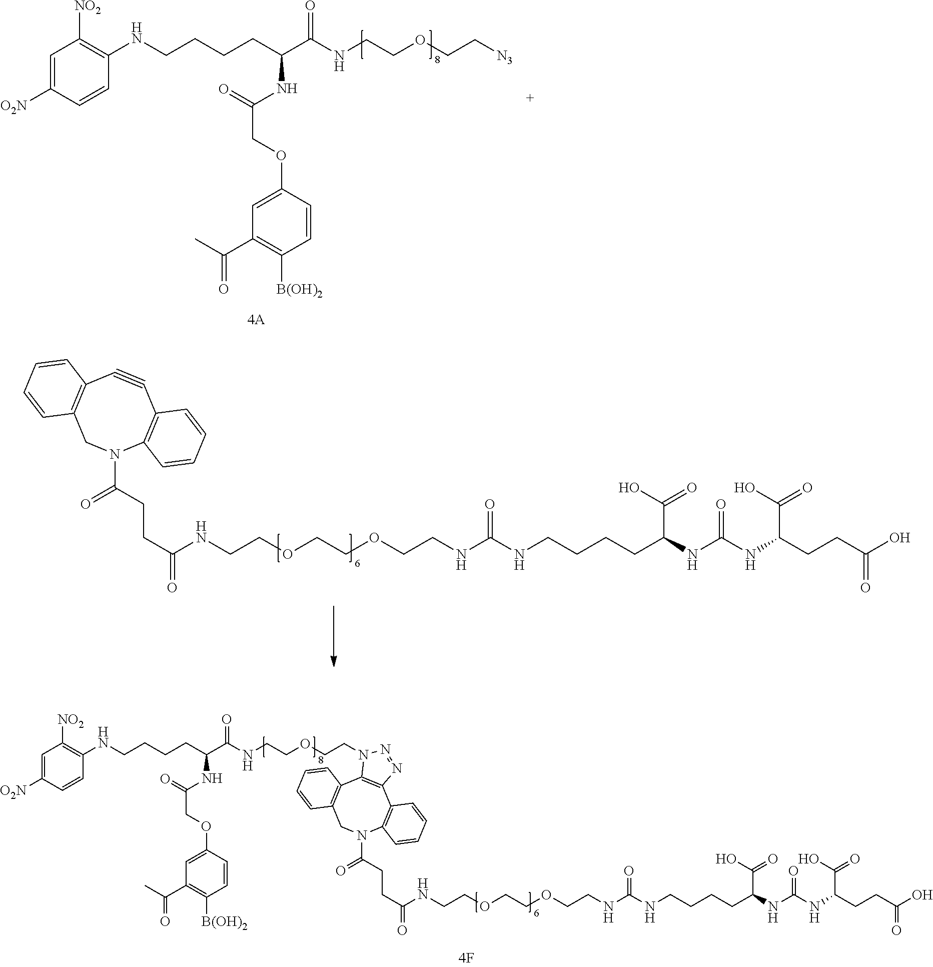

4A was combined 1:1 with DBCO-OEG₇-GUL in 10% DMSO:PBS at a final concentration of 100 μM and a final volume of 100 μL. The reaction was heated to 37° C. for 2 hrs. 4F was used without further purification. LCMS (ESI+) [M+3H]³⁺ m/z calc for [C₈₈H₁₃₂BN₁₄O₃₅]: 651.97, found 651.02.

### Synthesis of 6F (SuFEx-GUL)

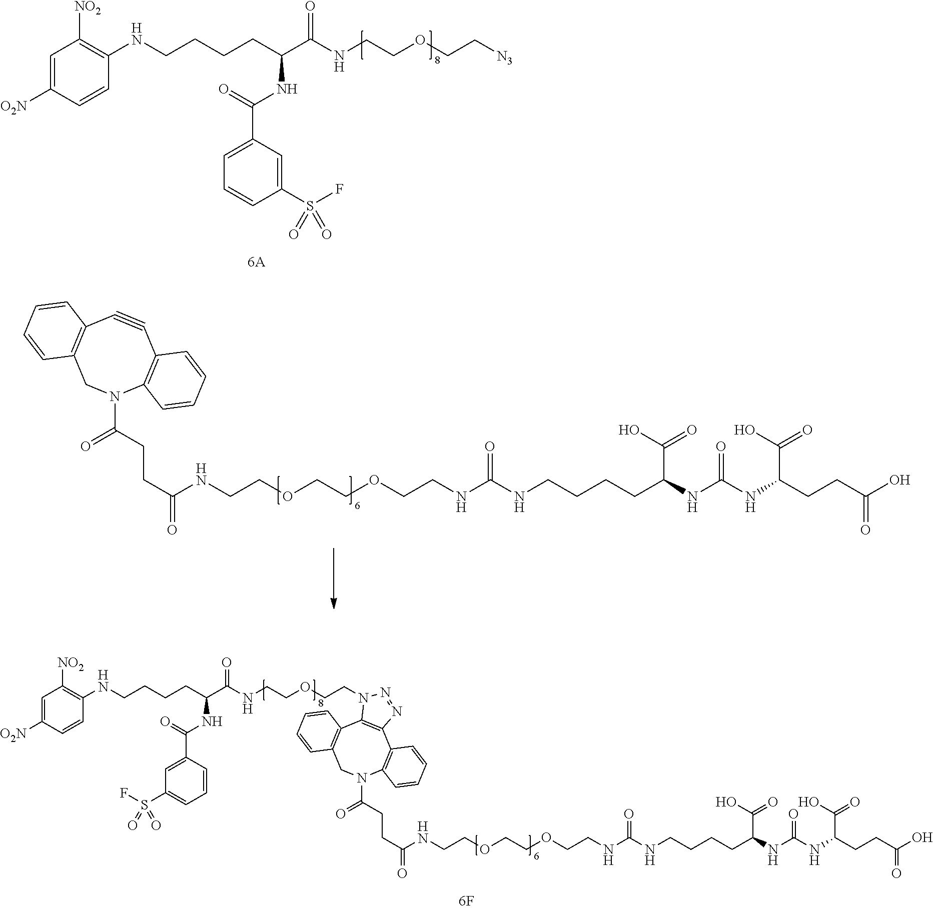

6A was combined 1:1 with DBCO-OEG₇-GUL in 10% DMSO:PBS at a final concentration of 100 μM and a final volume of 100 μL. The reaction was heated to 37° C. for 2 hrs. 6F was used without further purification. LCMS (electrospray ionization (ESI)+) [M+2H]2+ m/z calc for [C₈₅H₁₂₅FN₁₄O₃₃S]: 960.41, found 960.75.

### Synthesis of 1E (Ph-knottin)

### a) Synthesis of Int-2D

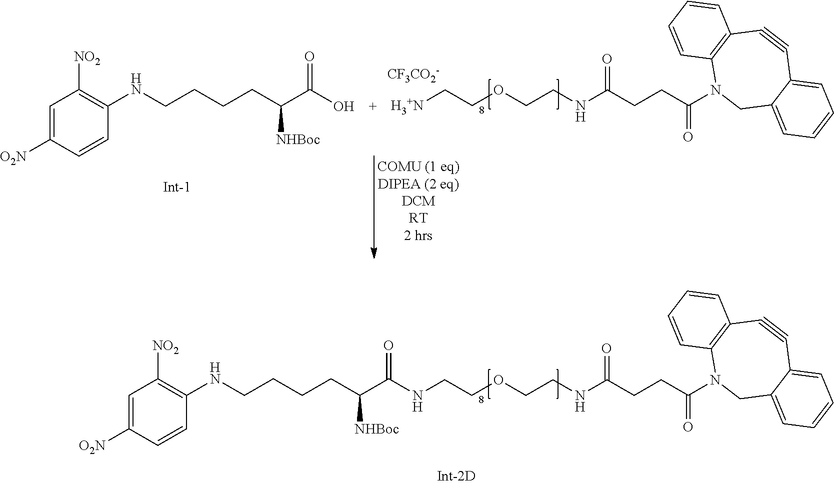

Int-1 (26 mg, 0.062 mmol, 1 eq) was dissolved in 1 mL of DCM along with DBCO-PEG₈-amine TFA salt (50 mg, 0.062 mmol, 1 eq) and N,N-diisopropylethylamine (16 mg, 0.124 mmol, 2 eq). The solution was stirred at room temperature, followed by the addition of COMU (27 mg, 0.062 mmol, 1 eq). The reaction was stirred for 2 hrs at room temperature. Solvent was removed in vacuo, and the crude material was purified with reverse phase chromatography with a C18 silica column (5:95 to 95:5 acetonitrile:water gradient) to yield Int-2D as a yellow oil. LCMS (ESI+) [M+H]⁺ m/z calc for [C₅₄H₇₆N₇O₁₇]: 1094.5290, found 1094.4550.

### b) Synthesis of Int-3D

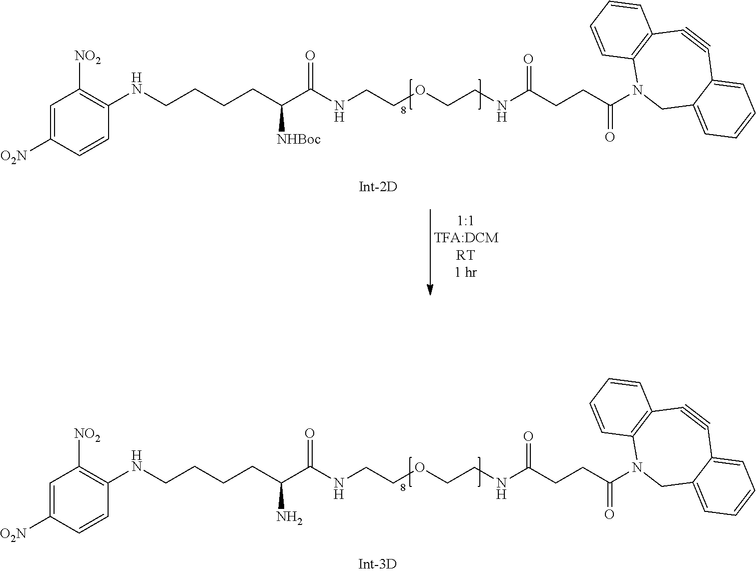

Int-2D (1 eq) was dissolved in 2 mL of 1:1 TFA:DCM and stirred for 1 hr at room temperature. Solvent was removed in vacuo and the crude product carried through to the next step.

### c) Synthesis of 10 (SEQ ID NO: 15)

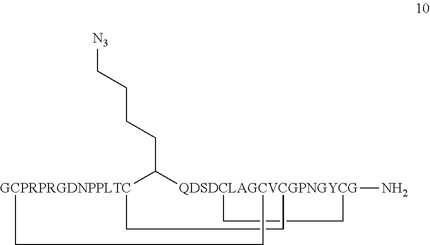

10 was synthesized on 0.1 mmol scale using Rink amide resin on a Chemical Engineering Manufacturing (CEM) Liberty Blue automated peptide synthesizer. Cleavage was performed at room temperature for 3 hours using 95:2.5:2.5 TFA:triisopropylsilane(TIS):water. The cleaved peptide was precipitated using ice cold diethyl ether, followed by drying and resuspension in DMSO for HPLC purification. After HPLC purification, the peptide was dissolved in 10 mL of folding cocktail (100 mM ammonium bicarbonate, 2.5 mM reduced glutathione, 60 mM NaOH, aq.) as described in reference. The mixture was then filtered, and the filtrate purified by HPLC to yield 10 (8.5 mg total yield). LCMS (ESI+) [M+2H]²⁺ m/z calc for [C₁₃₄H₂₀₈N₄₆O₄₅S₆]: 1687.8716, found 1688.1757.

### d) Synthesis of Int-1E

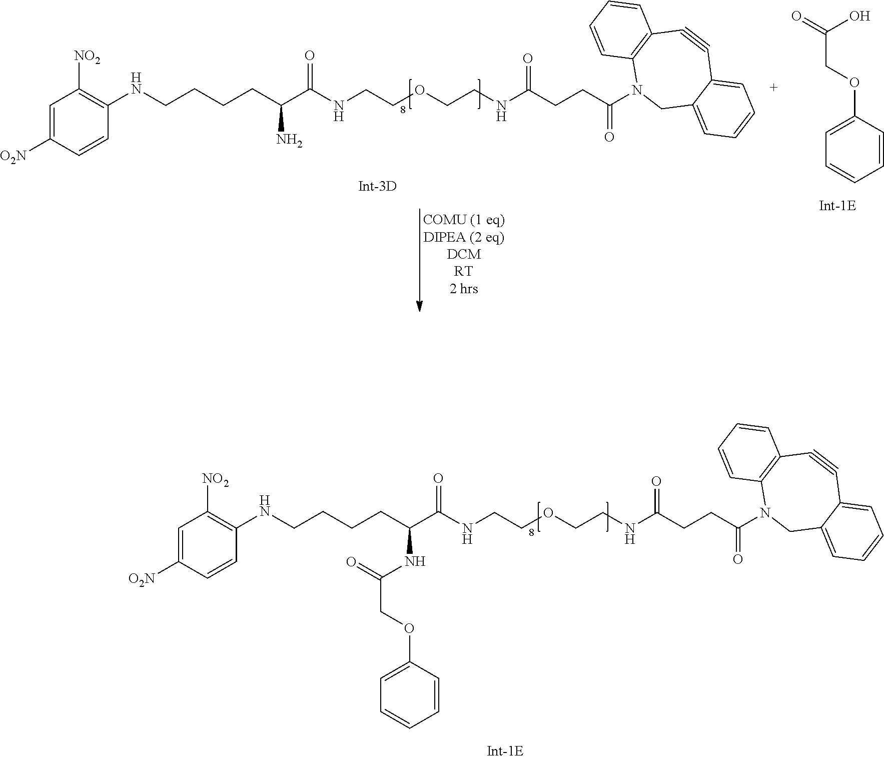

Int-3D was dissolved along with Int-5A and N,N-diisopropylethylamine in 1 mL DCM. Next, COMU was added and the reaction stirred for 2 hours at room temperature. Solvent was removed in vacuo and the crude mixture purified using HPLC. The resulting pure compound was dissolved in DMSO and concentrations were calculated by comparing absorbance at 360 nm to a DNP-glycine standard curve. Int-1E was obtained as a yellow oil. LCMS (ESI+) [M+H]⁺ m/z calc for [C₅₇H₇₄N₇O₁₇]: 1128.5136, found 1128.6846.

### e) Synthesis of 1E

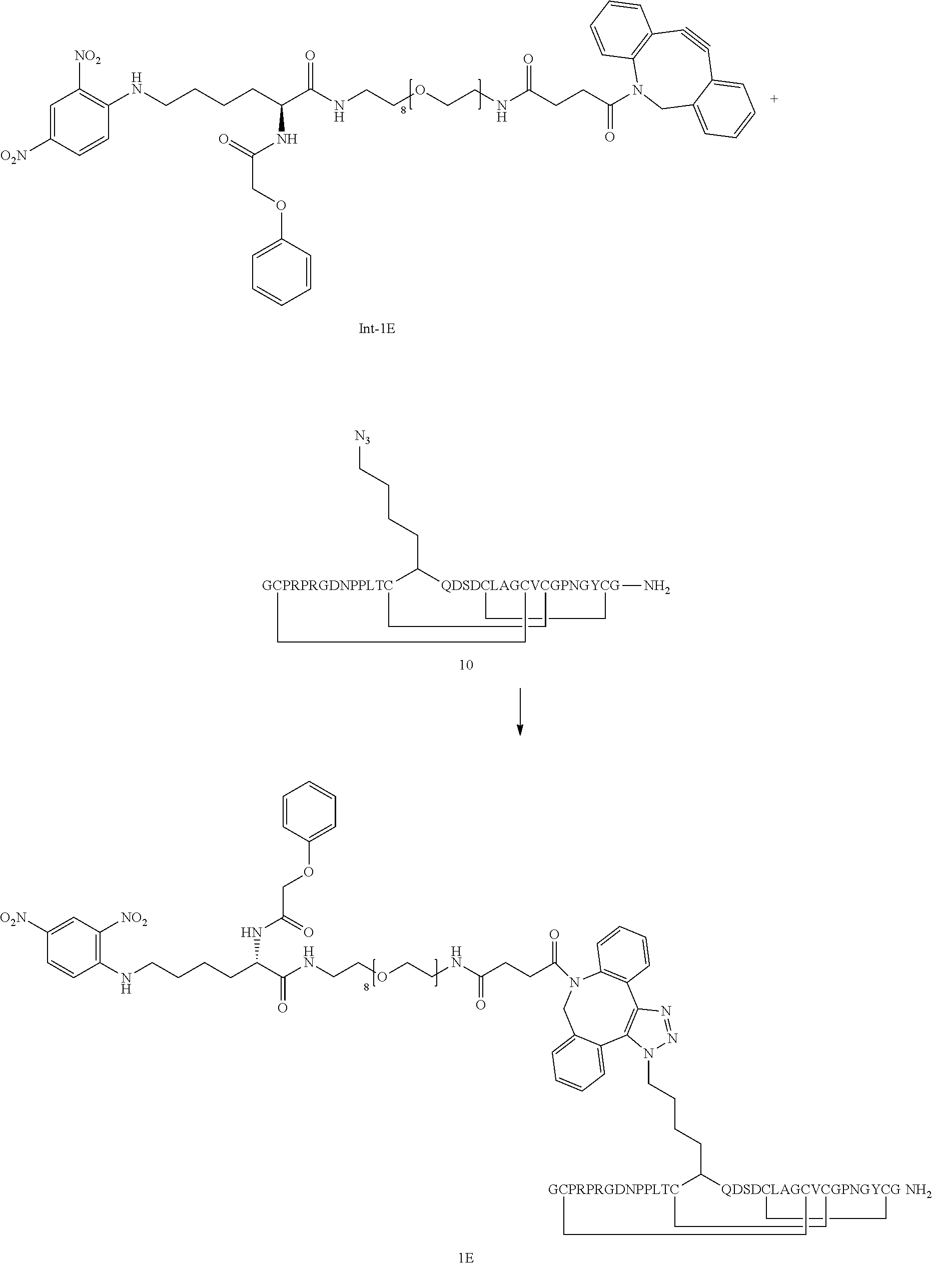

Int-1E was combined 1:1 with 10 in 40% DMSO:PBS at a final concentration of 500 μM and a final volume of 900 μL. The reaction was heated to 37° C. for 2 hrs, and lE was used without further purification. LCMS (ESI+) [M+3H]³⁺ m/z calc for [C₁₉₁H2₈₂N5₃06₂S6]: 1501.2973, found 1501.8928.

### Synthesis of 4E (2-APBA-knottin) (compound I-10)

### a) Synthesis of Int-7D

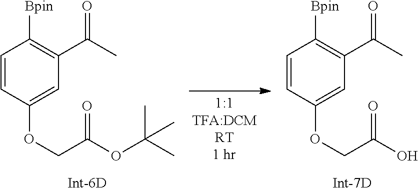

Int-6D (39.5 mg, 0.11 mmol, 1 eq) was dissolved in 2 mL of 1:1 TFA:DCM and stirred for 1 hr at room temperature. Solvent was removed in vacuo and the crude product carried through to the next step.

### b) Synthesis of Int-4F

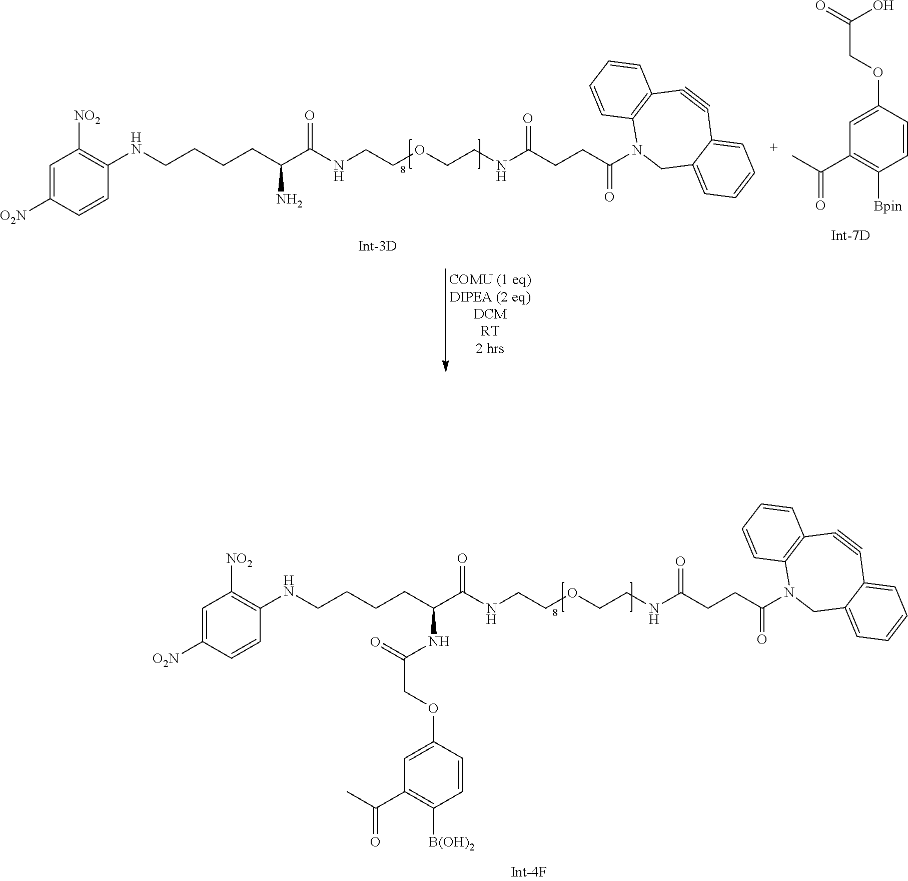

Int-3D was dissolved along with Int-7D and N,N-diisopropylethylamine in 1 mL DCM. Next, COMU was added and the reaction stirred for 2 hours at room temperature. Solvent was removed in vacuo and the crude mixture purified using HPLC. The resulting pure compound was dissolved in DMSO and concentrations were calculated by comparing absorbance at 360 nm to a N-(2,4-dinitrophenyl(DNP)-glycine standard curve. Int-4F was obtained as a yellow oil. LCMS (ESI+) [M+H]⁺ m/z calc for [C₅₉H77BN₇O₂₀]: 1214.5311, found 1214.5990.

### c) Synthesis of 4E

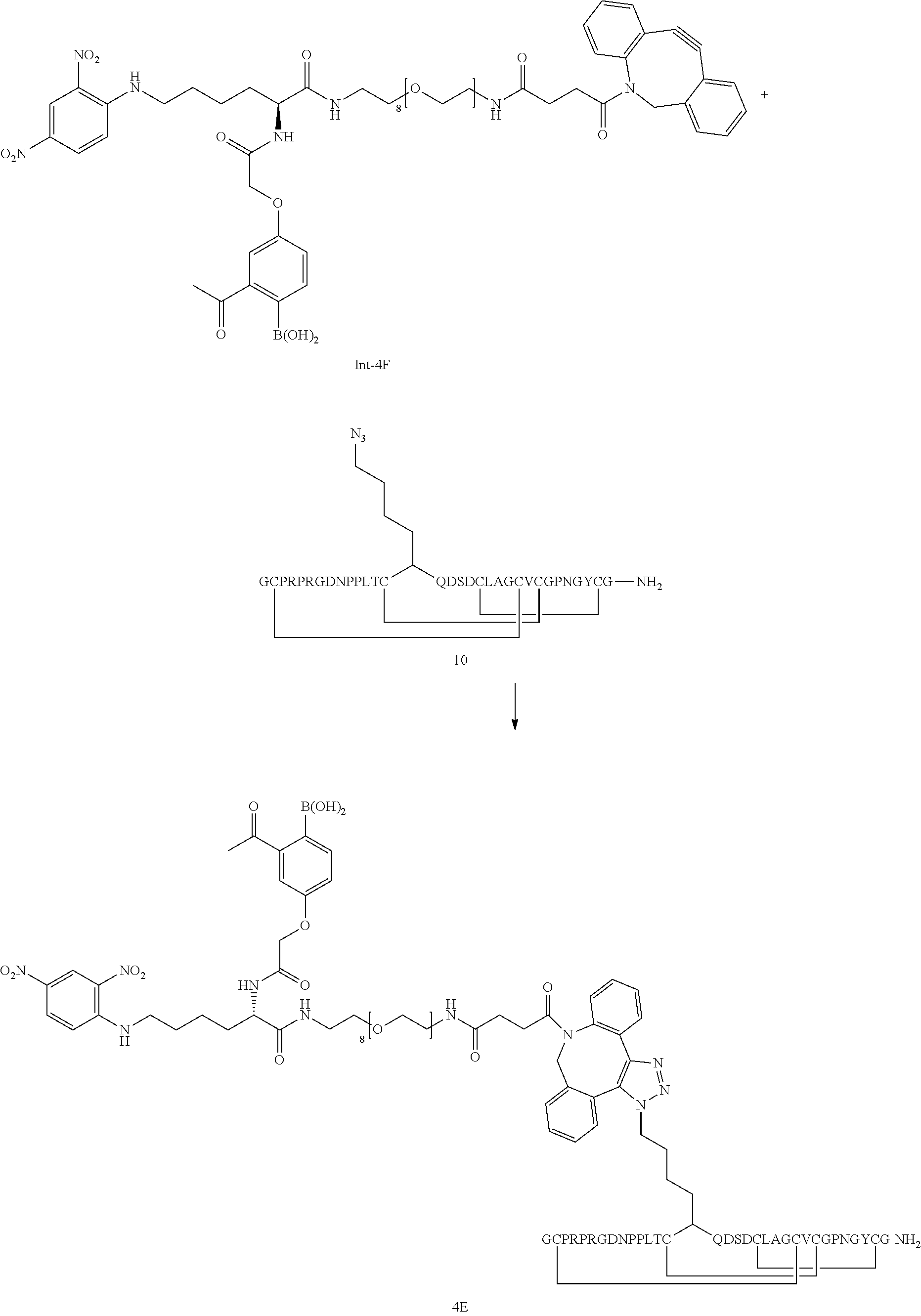

Int-4F was combined 1:1 with 10 in 40% DMSO:PBS at a final concentration of 500 μM and a final volume of 900 μL. The reaction was heated to 37° C. for 2 hrs, and 4E was used without further purification. LCMS (ESI+) [M+3H−H₂O]³⁺ m/z calc for [C₁₉₁H₂₈₃BN₅₃O₆₄S₆]: 1523.9662, found 1523.9232.

### Synthesis of 6E (SuFEx-knottin)

### a) Synthesis of Int-6F

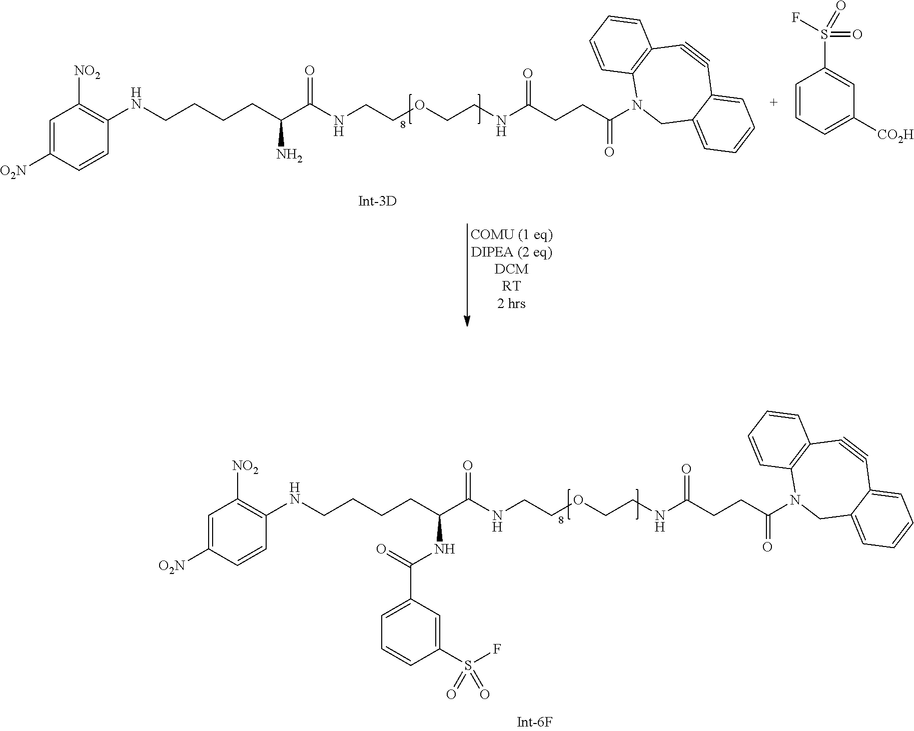

Int-3D was dissolved along with 3-fluorosulfonylbenzoic acid and N,N-diisopropylethylamine in 1 mL DCM. Next, COMU was added and the reaction stirred for 2 hours at room temperature. Solvent was removed in vacuo and the crude mixture purified using HPLC. The resulting pure compound was dissolved in DMSO and concentrations were calculated by comparing absorbance at 360 nm to a DNP-glycine standard curve. Int-6F was obtained as a yellow oil. LCMS (ESI+) [M+H]⁺ m/z calc for [C₅₆H₇₁FN₇O₁₈S]: 1180.4560, found 1180.6862.

### b) Synthesis of 6E

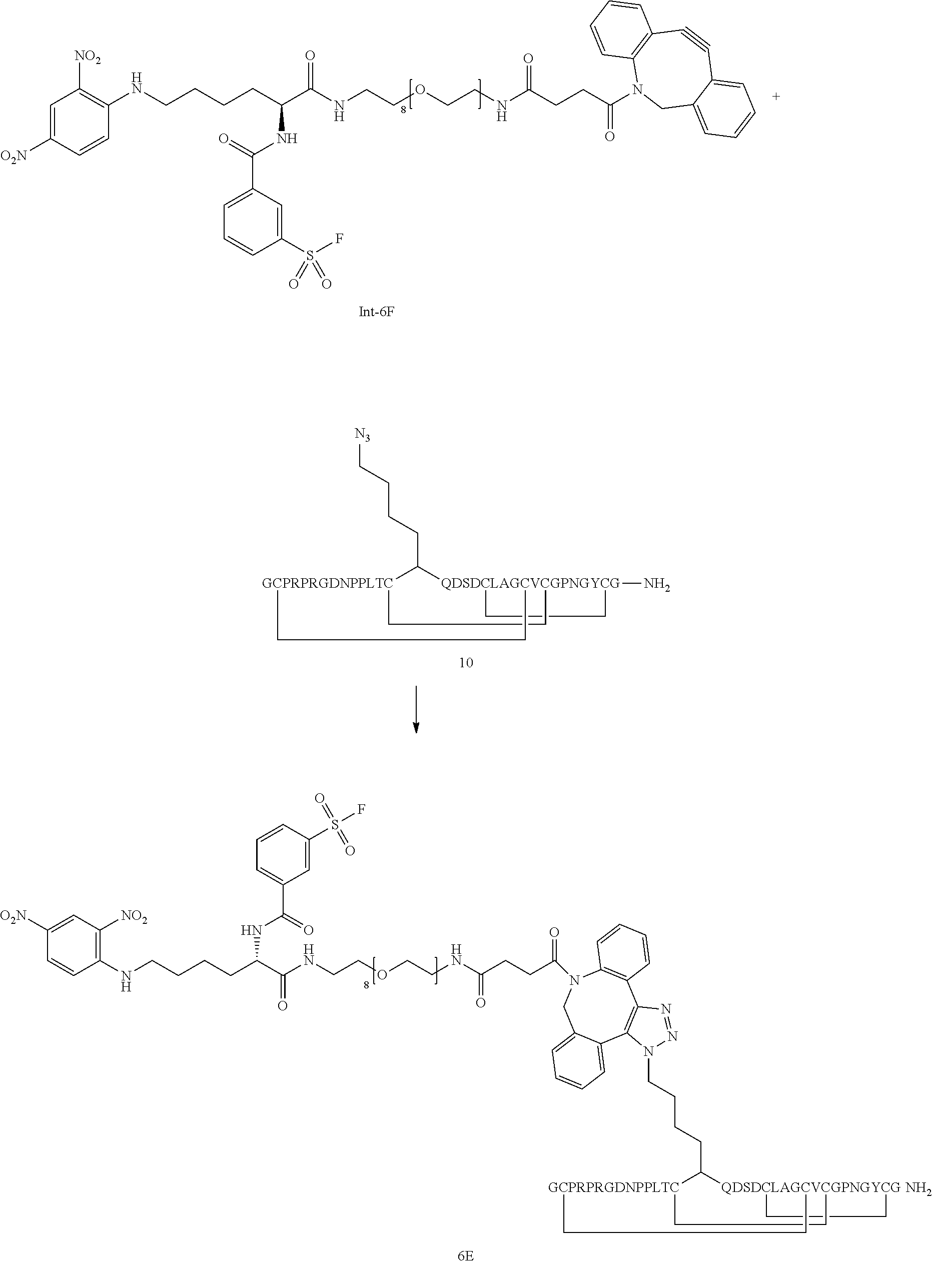

Int-6F was combined 1:1 with 10 in 40% DMSO:PBS at a final concentration of 500 μM and a final volume of 900 μL. The reaction was heated to 37° C. for 2 hrs. 6E could only be isolated as the —HF cyclization product. LCMS (ESI+) [M+3H−HF]³⁺ m/z calc for [C₁₉₀H₂₇₈N₅₃O₆₃S₇]: 1511.6080, found 1511.6769.

### Synthesis of rCGM-1 (compound I-11)

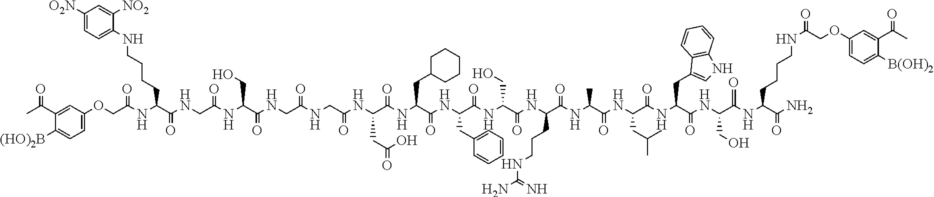 [SMILES: CC(=O)c1cc(OCC(=O)NCCCC[C@H](NC(=O)[C@H](CO)NC(=O)[C@H](Cc2c[nH]c3ccccc23)NC(=O)[C@H](CC(C)C)NC(=O)[C@H](C)NC(=O)[C@@H](CCCNC(=N)N)NC(=O)[C@@H](CO)NC(=O)[C@H](Cc2ccccc2)NC(=O)[C@H](CC2CCCCC2)NC(=O)[C@H](CC(=O)O)NC(=O)CNC(=O)CNC(=O)[C@H](CO)NC(=O)CNC(=O)[C@H](CCCCNc2ccc([N+](=O)[O-])cc2[N+](=O)[O-])NC(=O)COc2ccc(B(O)O)c(C(C)=O)c2)C(N)=O)ccc1B(O)O]

rCGM-1 was synthesized on 0.1 mmol scale using Rink amide resin on a CEM Liberty Blue automated peptide synthesizer. Cleavage was performed at room temperature for 3 hours using 95:2.5:2.5 TFA:TIS:water. The cleaved peptide was precipitated using ice cold diethyl ether, followed by drying and resuspension in DMSO for HPLC purification. rCGM-1 was obtained as a yellow solid and concentration measured with a 360 nm DNP-glycine calibration curve. LCMS (ESI+) [M+2H-2H2O]²⁺ m/z calc for [C₁₀₁H₁₃₆B₂N₂₄O₃₂]: 1109.9981, found 1110.3718.

### Synthesis of rCGM-2 (compound I-12)

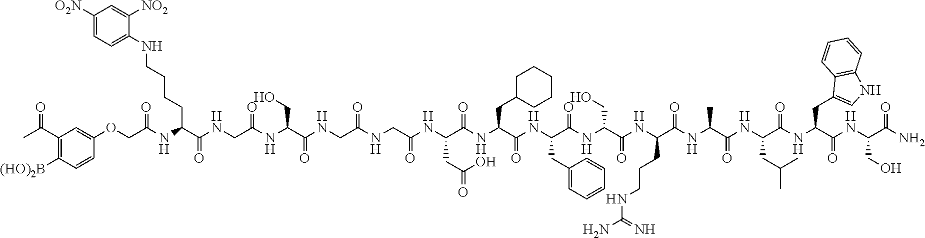 [SMILES: CC(=O)c1cc(OCC(=O)N[C@@H](CCCCNc2ccc([N+](=O)[O-])cc2[N+](=O)[O-])C(=O)NCC(=O)N[C@@H](CO)C(=O)NCC(=O)NCC(=O)N[C@@H](CC(=O)O)C(=O)N[C@@H](CC2CCCCC2)C(=O)N[C@@H](Cc2ccccc2)C(=O)N[C@H](CO)C(=O)N[C@H](CCCNC(=N)N)C(=O)N[C@@H](C)C(=O)N[C@@H](CC(C)C)C(=O)N[C@@H](Cc2c[nH]c3ccccc23)C(=O)N[C@@H](CO)C(N)=O)ccc1B(O)O]

rCGM-2 was synthesized on 0.1 mmol scale using Rink amide resin on a CEM Liberty Blue automated peptide synthesizer. Cleavage was performed at room temperature for 3 hours using 95:2.5:2.5 TFA:TIS:water. The cleaved peptide was precipitated using ice cold diethyl ether, followed by drying and resuspension in DMSO for HPLC purification. rCGM-2 was obtained as a yellow solid and concentration measured with a 360 nm DNP-glycine calibration curve. LCMS (ESI+) [M+H]⁺ m/z calc for [C₈₅H₁₁₈BN₂₂O₂₈]: 1905.8573, found 1906.4247.

### Synthesis of rCGM-3 (compound I-13)

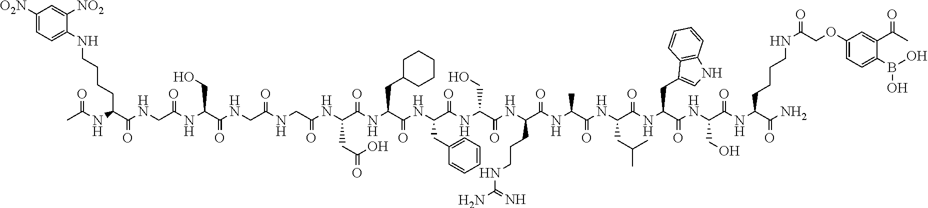 [SMILES: CC(=O)N[C@@H](CCCCNc1ccc([N+](=O)[O-])cc1[N+](=O)[O-])C(=O)NCC(=O)N[C@@H](CO)C(=O)NCC(=O)NCC(=O)N[C@@H](CC(=O)O)C(=O)N[C@@H](CC1CCCCC1)C(=O)N[C@@H](Cc1ccccc1)C(=O)N[C@H](CO)C(=O)N[C@H](CCCNC(=N)N)C(=O)N[C@@H](C)C(=O)N[C@@H](CC(C)C)C(=O)N[C@@H](Cc1c[nH]c2ccccc12)C(=O)N[C@@H](CO)C(=O)N[C@@H](CCCCNC(=O)COc1ccc(B(O)O)c(C(C)=O)c1)C(N)=O]

rCGM-3 was synthesized on 0.1 mmol scale using Rink amide resin on a CEM Liberty Blue automated peptide synthesizer. Cleavage was performed at room temperature for 3 hours using 95:2.5:2.5 TFA:TIS:water. The cleaved peptide was precipitated using ice cold diethyl ether, followed by drying and resuspension in DMSO for HPLC purification. rCGM-3 was obtained as a yellow solid and concentration measured with a 360 nm DNP-glycine calibration curve. LCMS (ESI+) [M+3H]3+ m/z calc for [C₉₃H₁₃₄BN₂₄O₃₀]: 692.6591, found 692.1825.

### Example 1. RPIMs Enable Reversible Covalent Binding to DNP-Specific Antibodies and Universal Synthetic Antigen Receptors

### Results

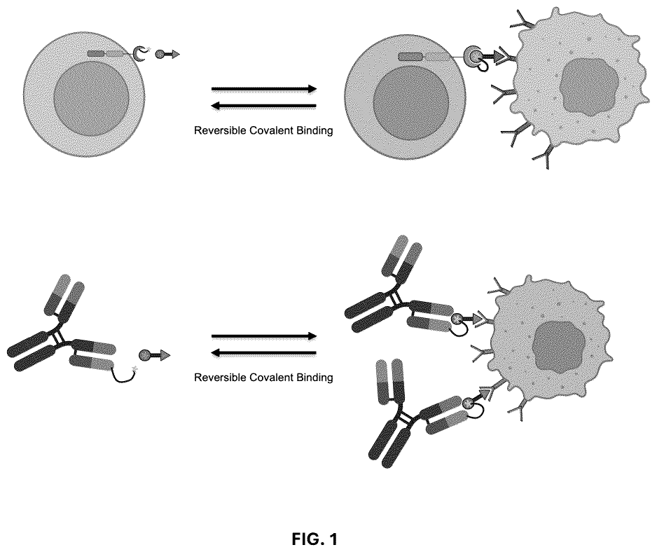

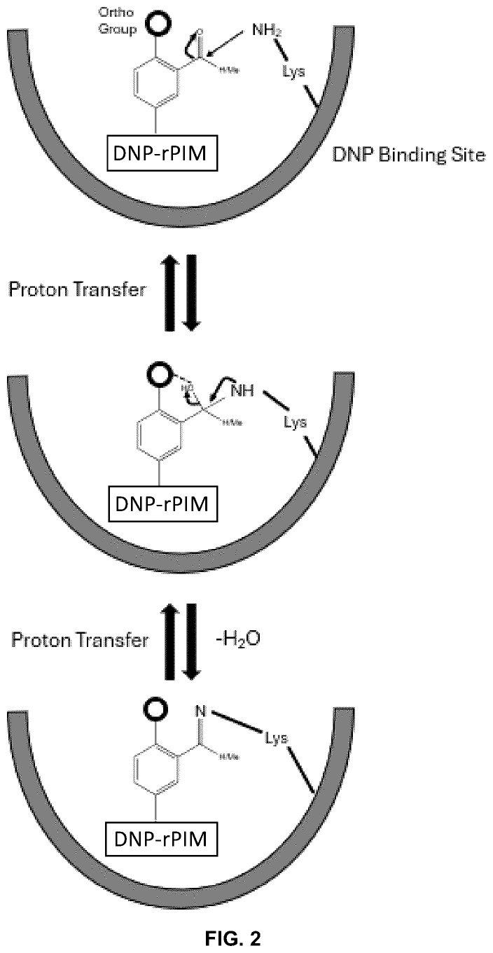

Proximity inducing molecules (PIMs) have shown effectiveness at enhancing function of universal synthetic antigen receptor (SAR) T cells and Fc mediated immune responses. Reversible covalent electrophiles in the context of PIMs that act as covalent reprogramming adapters as disclosed herein, allow for covalent anchoring in a similar manner to traditional PIMs, but also benefit from reversibility which, in principle, should enable the rPIM to release from a given ligand and bind to another. Further, the reversible nature of the rPIM protects the molecule from possible hydrolysis in biological fluids and increase the overall potency. FIG. 1 shows these rPIMs functioning as synthetic molecular adapters, comprising donors that bind a serum antibody or universal SAR and tumor ligands, couple the immune cell to the target cell. The universal SAR shown herein is introduced into T cells via gene transfer and expressed on the cell surface. As a proof of concept, DNP was utilized as a donor molecule to target both anti-DNP antibodies and anti-DNP scFv containing universal engineered T cells for labelling via the reversible electrophilic chemistries. Once the DNP guided rPIM binds DNP in its binding pocket, the reversible electrophilic group is placed in close proximity to a nucleophilic lysine in the binding pocket (FIG. 2). Nucleophilic attack at the ketone or aldehyde of the CBD results in a condensation reaction which can be stabilized via coordination of the group ortho to the electrophile in the aromatic ring. The resulting imine forms a covalent bond anchoring the rPIM to the protein. This reaction is then reversible to allow for release of the rPIM. If present, the coordination from the ortho group both facilitates imine formation and the reverse reaction which converts the covalently attached molecule back to the starting product. The ortho group can be interchanged to tune the reaction kinetics for optimal function. Upon the reverse reaction, the rPIM reforms the original reactive molecule, a function not attained with traditional PIMs.

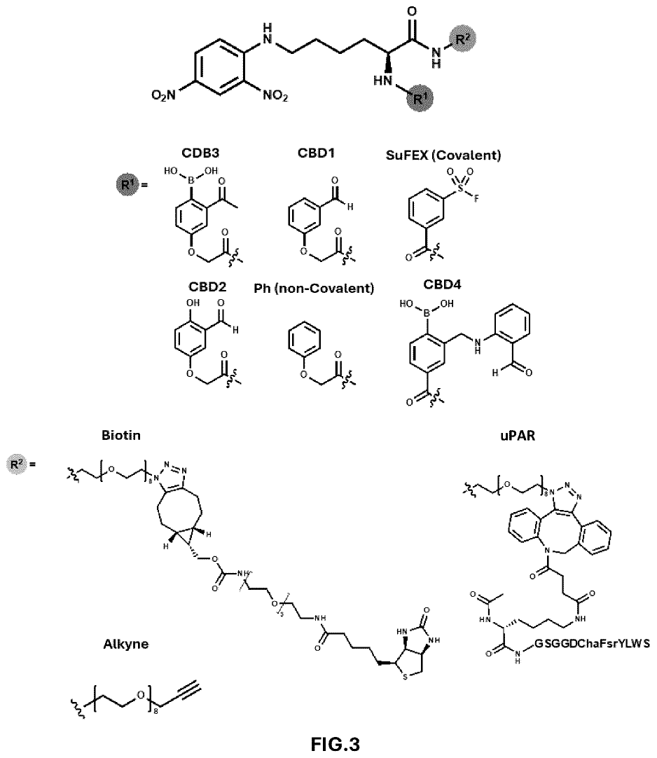

Four reversible covalent electrophilic CBDs (CBD1, CBD2, CBD3 and CBD4) were designed (FIG. 3) and functionalized with biotin (compounds I-5 (CBD1), I-6 (CBD2), I-7 (CBD3) and I-8 (CBD4)) or a uPAR (compounds I-1 (CBD1), I-2 (CBD2), I-3 (CBD3) and I-4 (CBD4)) binding peptide via linker groups for targeting uPAR expressed on various cancer cell lines, as described above. This library of rPIMs was developed off a single backbone. The electrophilic reactive groups “R¹” feature aldehydes and ketones, optionally with different ortho substituted functional groups. The warhead scaffold of the rPIMs were synthesized with both alkyne and azide click handles in position “R²” to allow facile and modular linking with multiple target domains (e.g. biotin-rPIM and uPAR-rPIM) to allow for easy library creation of different targeting moieties using click reactions. Additionally, a matched irreversible covalent (SuFEX) PIM and a non-covalent PIM ((N) PIM), referred to as “Ph”, was prepared for comparison (FIG. 3).

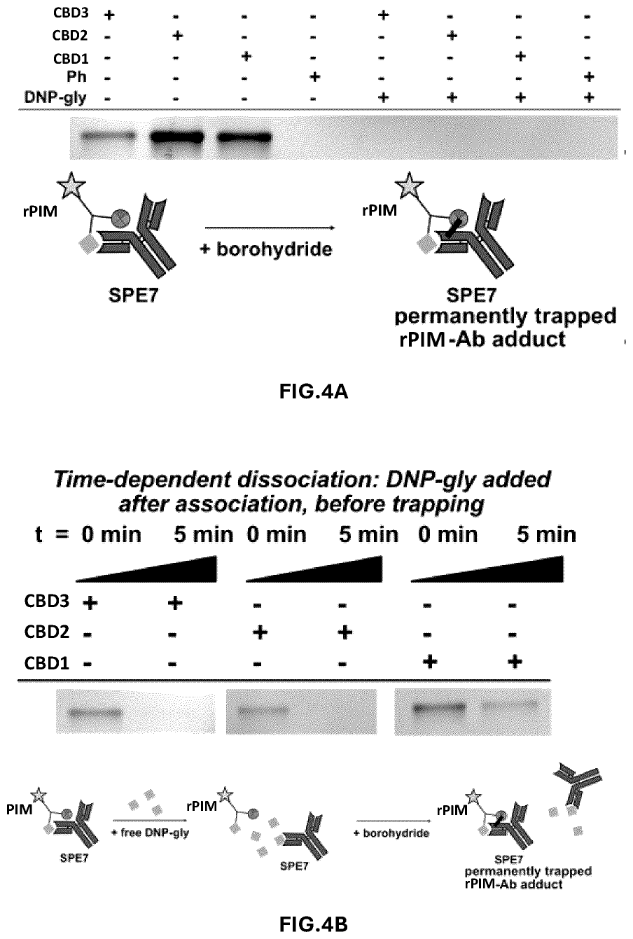

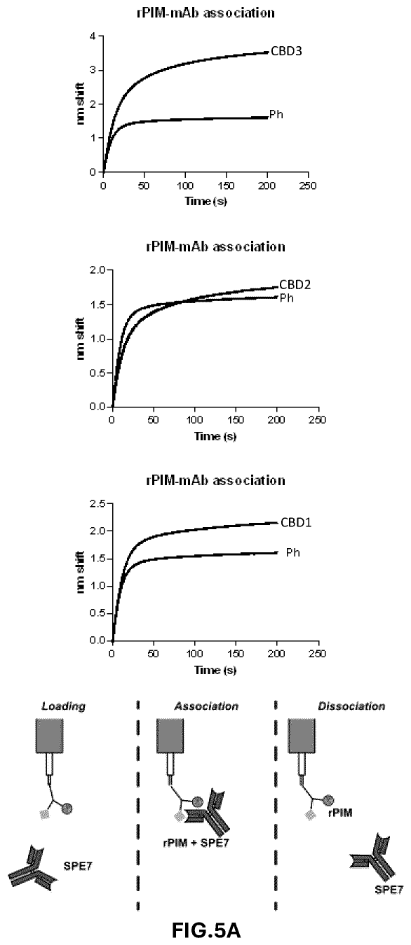

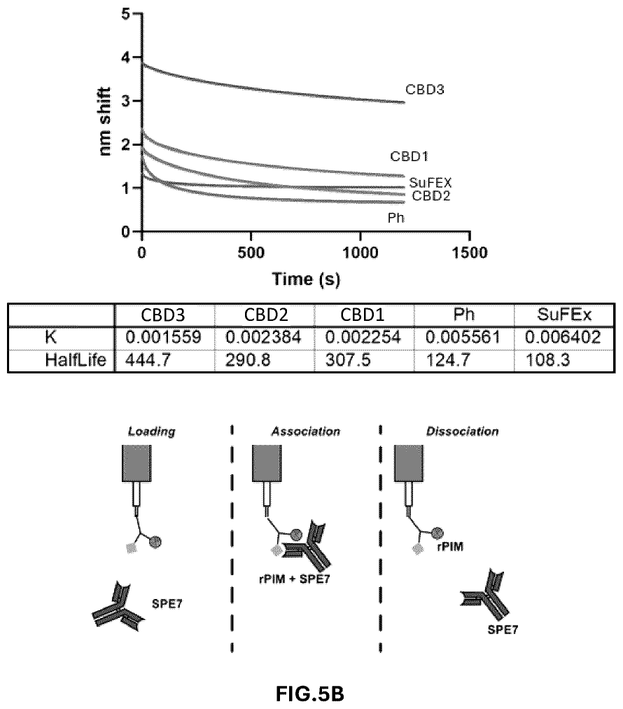

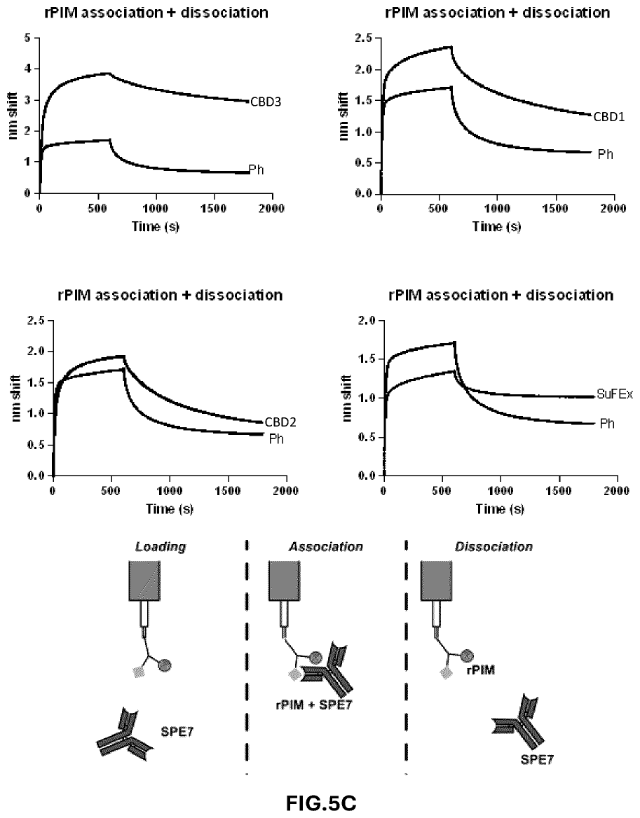

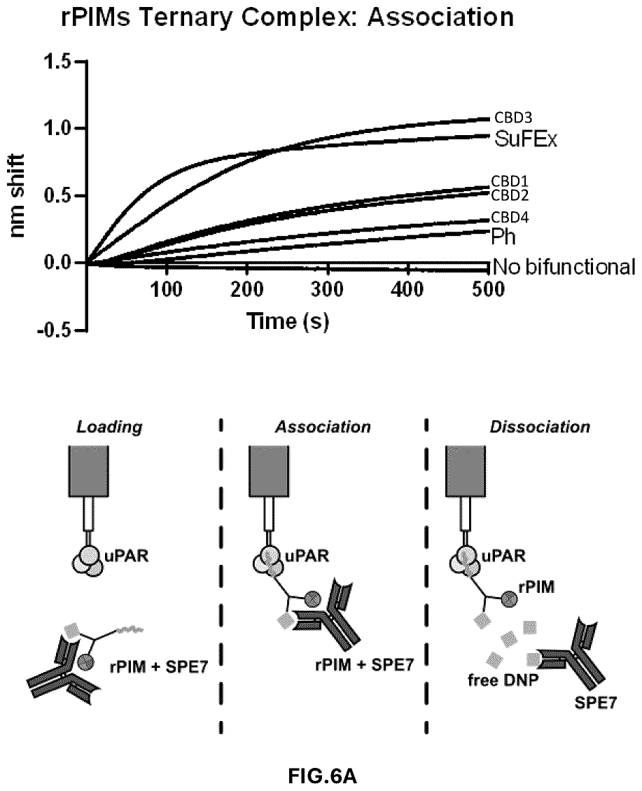

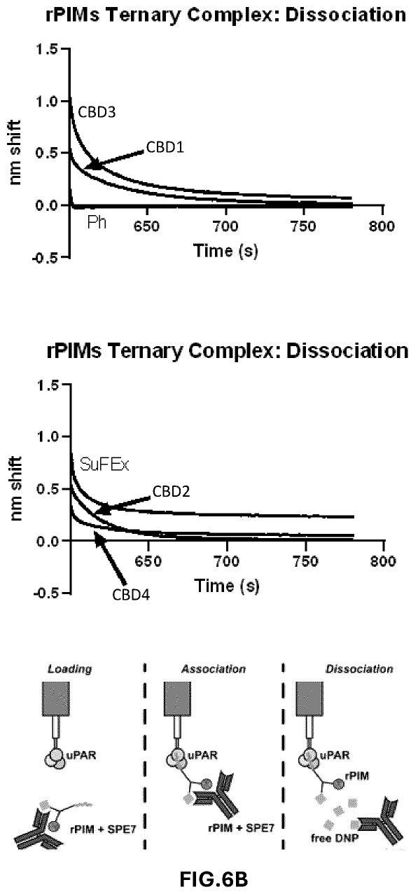

Using anti-DNP monoclonal antibody (mAb), it was shown that all uPAR rPIM molecules are capable of labelling the anti-DNP mAb with a specificity that can be outcompeted by excess free DNP (DNP-gly) (FIG. 4A). The non-covalent bifunctional agent, labeled “Ph” is not retained, which demonstrates the importance of the covalent linkage. Further, dissociation or reversibility of the interaction was shown by labelling anti-DNP mAb with the rPIMs (at t=0 min) followed by incubation in the presence of excess DNP-gly, which resulted in removal of the reversible label (FIG. 4B). By changing the reversible electrophile, the binding kinetics of the rPIM-protein interaction can be altered. Biolayer interferometry (BLI) results showed that the various rPIMs have differing reaction kinetics, properties which will have impacts downstream on effector function (FIGS. 5A-5C). The results demonstrate that the association rates for the imine-forming rPIMs are ordered CBD3>CBD2>CBD1 (FIG. 5B), while the dissociation half-lives of rPIMs vary based on structure, in the order CBD3>CBD1>CBD2>Ph (Table 1). Further, the ability for the rPIMs to induce antibody-rPIM-target protein binding in a ternary complex was assessed via BLI, a property which is unique to each electrophile (FIG. 6A-6B). The results demonstrate that the different uPAR-rPIMs (compounds I-1, 1-2, 1-3 and 1-4) form varying amounts of ternary complex (uPAR:rPIM:anti-DNP) (FIG. 6A). These ternary complexes have differing dissociation half-lives in the presence of DNP-gly competitor (FIG. 6B), which again follow the order CBD3>CBD1>CBD2>Ph.

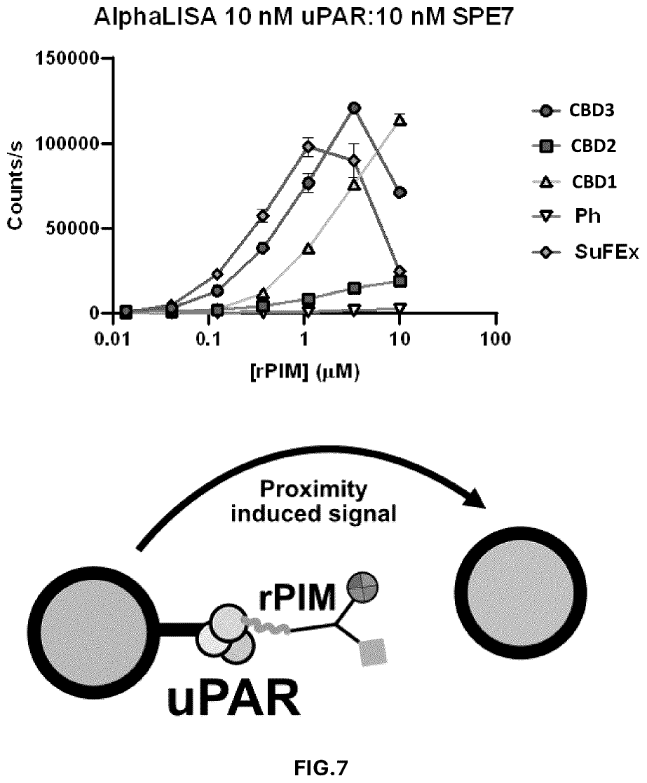

This artificial ternary complex between immune component and target protein is a factor for rPIM-induced immune function. Using AlphaLISA as a bead-based ternary complex assay, the ability of rPIMs to bridge between mAb and uPAR-functionalized surfaces was validated (FIG. 7). Acceptor and donor beads functionalized with anti-DNP mAb and uPAR were brought together by the different rPIMs. The results demonstrate that rPIMs induce differing amounts of bead-bead interactions. Ternary complex potency follows the order SuFEx >CBD3>CBD1>CBD2>Ph. It was found that CBD3 forms the maximum amount of ternary complex at μM concentrations. Again, ternary complex formation is electrophile dependent, with CBD3 and SuFEx showing the greatest potency and total complex. CBD3 and SuFEx also display the autoinhibition/hook effect at high concentrations that is characteristic of ternary complex binding.

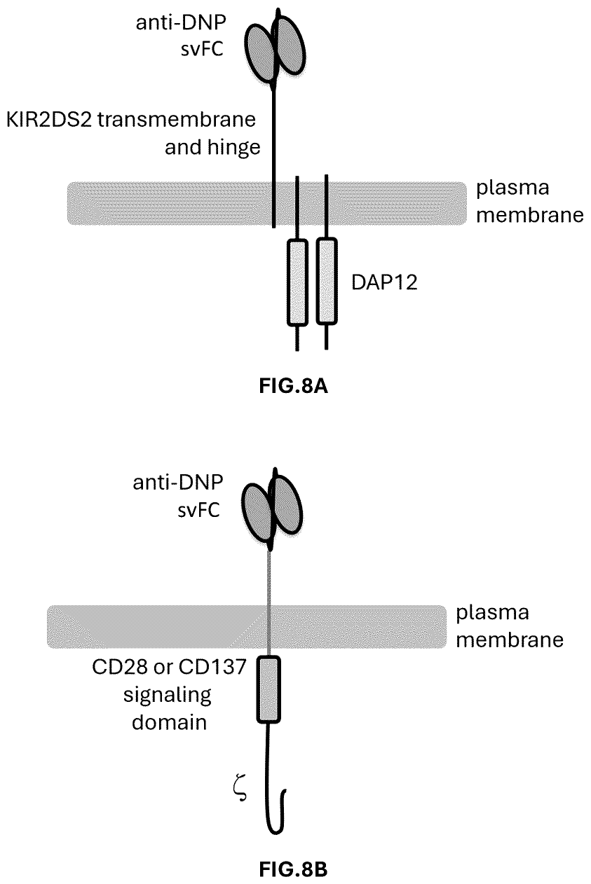

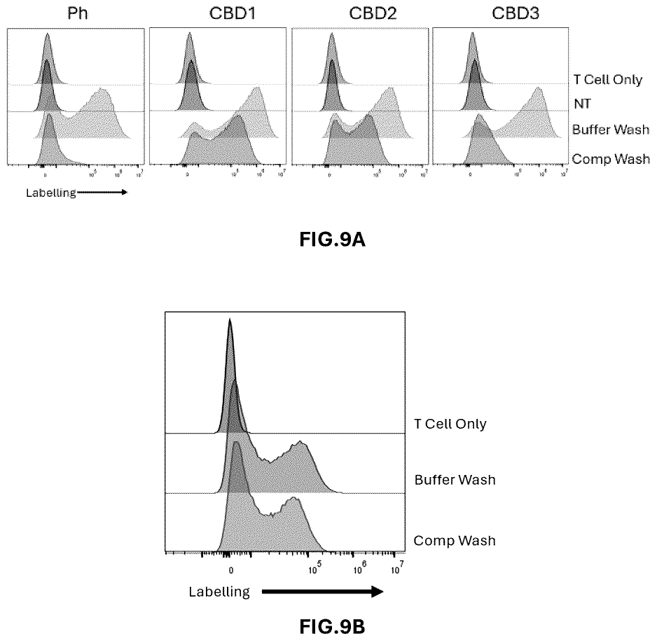

Beyond antibodies, it was shown that these DNP-rPIMs can be used with anti-DNP KIR-CAR (FIG. 8A), CD28-CAR and CD137-CAR (FIG. 8B) T cells. The DAP12-based SAR (KIR-CAR) is comprised of the anti-DNP scFv in VH-VL orientation linked to the transmembrane and extracellular domain of KIR2DS2. The chimeric antigen receptor (CAR) is a single polypeptide that comprises the anti-DNP scFv in VH-VL configuration, an intracellular costimulatory signaling domain derived from CD28 or CD137, and the cytoplasmic portion of CD3(. Expression on primary T cells was confirmed by flow cytometry. The ability of the rPIMs to label anti-DNP scFv containing KIR-CAR T cells was demonstrated by the selective and reversible binding of the biotin DNP-rPIMs (compounds 1-5, 1-6, 1-7 or 1-8) to T cells engineered with the DNP—KIR-CAR (FIG. 9A). Anti-DNP KIR-CAR and non-transduced (NT) T cells were incubated with rPIMs containing the various CBD groups (i.e. Ph, CBD1, CBD2, CBD3). Cells were then washed in buffer alone (Buffer Wash) or with DNP competitor added to the buffer wash (Comp Wash) and stained by flow cytometry. All rPIMs and the (N)PIM (Ph) remain attached following wash in buffer. The CBD3, CBDland CBD2 rPIMs, which have reversible chemistries, are removed to varying degrees when competitor is included in the wash buffer, showing that similar to the labeling of anti-DNP antibodies, the rPIMs were removed from the labeled T cells by washing the cells in the presence of excess DNP; the (N)PIM, Ph, was completely removed when competitor was included in the wash (FIG. 9A). Cells labeled with a PIM that leads to irreversible/permanent covalent attachment (SuFEX PIM) were not displaced from the T cells when washed in the presence of DNP-gly in the wash buffer, demonstrating this ability to unbind is unique to the rPIMs (FIG. 9B).

### Methods

SDS-PAGE: Alkyne-rPIMs (2 μM) were incubated with anti-DNP mAb (1 μM) for 1 hour at room temperature. Additional conditions included 200 μM DNP glycine as a competitor, added either before or after rPIM-Ab incubation. Sodium borohydride (1.5 mM) was added for 1 hr to reductively trap rPIM-Ab imine linkages. Reactions were purified with a 7 k molecular weight cut off (MWCO) spin column. Next, 100 μM of BPfluor-488-azide was added, along with 1 mM copper (II) sulfate (CuSO₄), 10 mM tris(3-hydroxypropyltriazolylmethyl)amine (THPTA), and 20 mM sodium ascorbate. After a 2-hr incubation at room temperature, the samples were diluted in 2X Laemmli buffer and run on a polyacrylamide gel for 2 hrs at 120 V. Gels were imaged on the Cy2 channel of a Typhoon imaging system.

Bio-layer interferometry: All BLI experiments were run on an Octet RED96 instrument (ForteBio). Streptavidin biosensors (Sartorius, item no. 18-5019) were dipped in 1X Octet kinetics buffer (Sartorius, Item No. 18-1105) for 60s to establish a stable baseline. Next, biotin-rPIMs (100 nM) were loaded onto streptavidin probes for 60s, followed by a 60s baseline. The loaded probes were then dipped into a solution containing anti-DNP mAb (250 nM) for 600s. The probes were then dipped into 1X Octet kinetics buffer for 600s. Dissociation kinetics were fit using one-phase exponential decay, and association kinetics were fit using two-phase association (GraphPad Prism 8.4.2).

Ternary complex BLIassay: Streptavidin biosensors were dipped in 1X Octet kinetics buffer for 60s to establish a stable baseline. Next, probes were dipped into a solution of recombinant biotin-uPAR (25 nM) for 180s of loading, followed by a 180s baseline in 1X Octet kinetics buffer. The loaded probes were then dipped into a solution containing a mixture of rPIM (200 nM) and anti-DNP mAb (100 nM) for 600s. The rPIM and anti-DNP mAb mixture was allowed to incubate for 30 minutes at room temperature prior to its use in this assay. The probes were then dipped into a solution containing 200 μM of DNP-glycine competitor for 180s.

AlphaLISA ternary complex assay: Varying concentrations of rPIM were incubated with 10 nM of recombinant uPAR-biotin and 10 nM of SPE7 mAb for 1 hr in 1X immunoassay buffer (Revvity, part no. ALOOOC) to allow for ternary complex formation. 10 μg/mL of AlphaLISA Anti-Human IgG (Fc specific) Acceptor Beads (Revvity, part no. AL103C) were added and the solutions incubated in the dark for 1 hr. Next, 10 μg/mL of AlphaScreen Streptavidin Donor Beads (Revvity, part no. 6760002S) were added and the solutions incubated in the dark for 1 hr. The incubations were transferred to a 384-well AlphaPlate (Revvity, part no. 6008350) and read on a TECAN Spark plate reader with an Alpha Technology add-on. Default wavelengths, excitations, and integration times were used.

Labeling with PIM (N) PIM: af3 T-cells were labelled for 2 hours at room temperature in T-cell media with 1 μM of the various Biotin rPIMs, Biotin-SuFEX PIM, or Biotin Ph (N) PIM. Cells were then washed with FACs buffer (2 mM EDTA, 0.5% BSA, in PBS) to remove non bound rPIM. As a control, some cells were washed with DNP-Glycine competitor at 200 μM in FACs buffer to display reversibility. Samples were then stained with Streptavidin-phycoerythrin (PE) to assess labelling. The samples were also stained with near IR Live/Dead, anti-CD4 Pacific Blue, and anti-CD8 Alexa fluor 700 to allow for appropriate gating on live a3 T-cells. The samples were assessed for Streptavidin-PE labelling via flow cytometry.

### Example 2. RPIM Reversible Covalent Binding Allows for Activation and Tumoricidal Function of Anti-DNP Universal T Cells and Monocytes

### Results

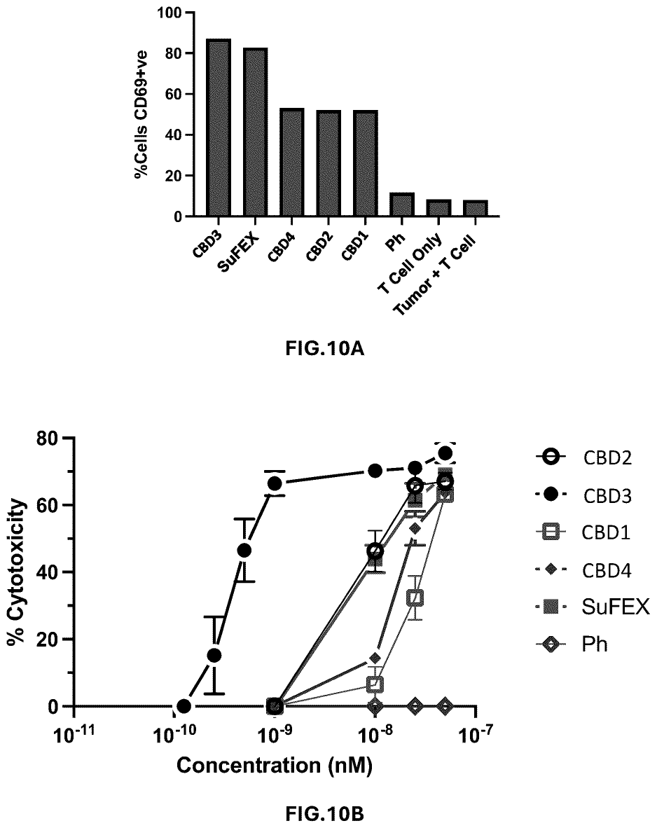

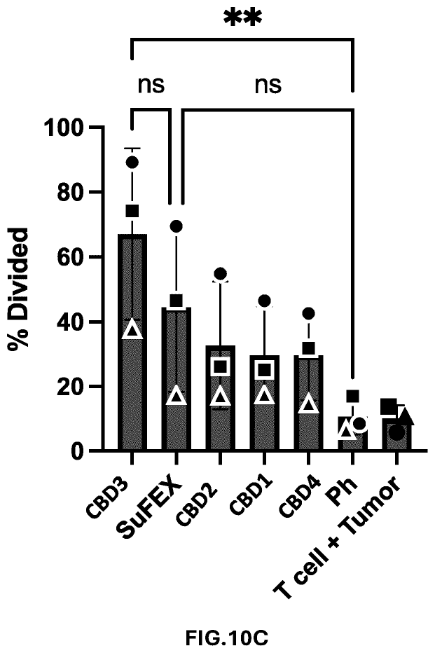

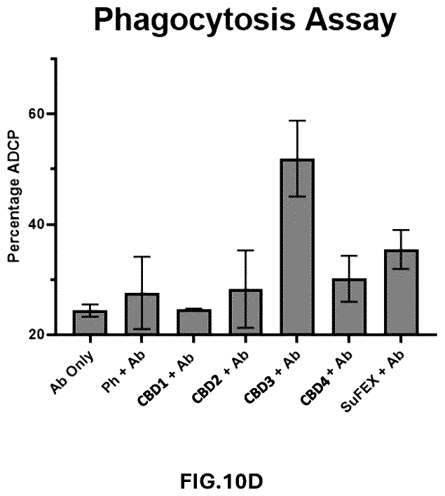

Tuning reversible covalency for use in functional reprogramming of cancer immunotherapeutics to date has not been previously tested. Engineered universal anti-DNP KIR-CAR T cells are one class of cancer immunotherapeutic with downstream clinical applications that are used herein to assess this library of compounds and their functionality. The functional advantage of rPIM reversible covalency with universal KIR-CAR T cells and Fc receptor mediated effector function is shown in FIGS. 10A-D. Upon bridging of the T cell to a tumor cell, CD69 expression can be observed as an early marker of activation. The ability for the library of the uPAR rPIMs (compounds I-1, I-2, I-3 or I-4) to facilitate T cell activation in the presence of uPAR expressing A172 tumor cells was assessed through flow cytometry by the upregulation of CD69. It was observed that T cells engineered with an SAR specific for DNP (DNP KIR-CAR T cells)¹ could be reprogrammed to become activated in response to uPAR expressing A172 glioblastoma cells via the uPAR targeted rPIMs (FIG. 10A). In this assay of T cell activation, the CBD3 rPIM (compound I-3) displayed the highest level of T cell activation. Further, killing of uPAR-expressing A172 glioblastoma cancer cells was assessed using DNP—KIR-CAR T cells co-cultured with A172 cells in the presence of varying concentrations of uPAR-rPIM and uPAR-(N) PIM, and uPAR SuFEX PIM. When assessing cytotoxicity against A172, all rPIMs were able to elicit some degree of cytotoxicity but to varying effectiveness, whereas the non-covalent control was unable to mediate cytotoxicity (FIG. 10B). The CBD3 rPIM again elicited the strongest cytotoxic function, even at very low concentrations (μM), far exceeding the cytotoxicity achieved with the non-reversible covalent SuFEX PIM. As the data show, some element of covalency is required for T cell activation and cytotoxicity as the non-covalent Ph molecule fails to elicit either function at any concentration. The capacity of the rPIMs and PIM to promote T cell proliferation was also assessed (FIG. 10C). Both PIM (SuFEX) and rPIMs (CBD3, CBD2, CBD1, CBD4) could mediate some degree of proliferation of the engineered T cells, with the CBD3 rPIM facilitating the highest proliferation across three PBMC donors compared to the irreversible covalent SuFEX PIM. The non-covalent adapter (Ph) fails to elicit any proliferation. As observed with the other effector readouts, the CBD3 rPIM produced the strongest response.

Reprogramming of serum antibodies is another avenue for use of the rPIMs. Reprogramming of antibodies could allow Fc mediated antibody dependent cellular phagocytosis (ADCP) by effector cells, like macrophages, which sense the polyvalent display of antibodies on a tumor cell generated via rPIM reprogramming. To test this, monocytes and A172 tumor cells were cultured in the presence of the rPIMs and PIM. Monoclonal anti-DNP (anti-DNP mAb) was also included in the co-culture to bridge the rPIM/PIM-bound tumor cells and Fc-expressing monocytes. ADCP was assessed by flow cytometry. Antibody only without uPAR-rPIM was added as a control to account for non rPIM directed phagocytosis. Only the CBD3 rPIM (compound I-3) facilitated significant ADCP above baseline, with only some function seen with all other PIMs (FIG. 10D).

### Methods

CD69 Activation Assessments: 2.5×10⁵ A172 tumor cells were seeded the day before the activation assay. 5×10⁵ engineered anti-DNP KIR-CAR T cells were added per well with 100 nM of uPAR rPIM, uPAR SuFEX PIM, uPAR Ph (N) PIM or media alone for 4 hours at 37° C. and 5% CO₂. Cell were subsequently collected, and stained with Pacific Blue-conjugated mouse anti-human CD4 (Cat No. 558116, BD Pharmingen), and AlexaFluor™700-conjugated mouse anti-human CD8a (Cat No. 56-0086-82, Invitrogen), and anti-huCD69 (Cat No. 563835, BD Horizon). The antibody-stained cells were subsequently analyzed using the Cytoflex™ LX flow cytometer.

A-172 T Cell Cytotoxicity Assay: A172 cells glioblastoma cells were engineered with eGFP lentivirus made in house. In these experiments, 5×10³ tumor cells per well were pre-plated in a 96 well flatbottom plate overnight. The next day anti-DNP KIR-CAR ap T cells were added to the tumor cells at an effector to target ratio of 8:1. Also added to the wells were the appropriate tumor targeting rPIMs molecules at various concentrations (0-50 nM). The three components were co-cultured for 4 days at 37° C. and 5% C02 in the Sartorius Incucyte S3 Live cell imaging system with 9 images per well taken every 8 hours. Green image mean for each image was used to determine tumor cell growth. The area under the growth curve (AUC) was analyzed using PRISM Graphpad and used as a metric for tumor cell growth for this data. The larger the area, the greater the tumor cell growth that occurred over the incubation period. The area under the curve for the tumor alone control and each condition were used to calculate the % cytotoxicity. Percent cytotoxicity was calculated as: % Cytotoxicity=((AUC Tumor Alone-AUC Sample)/(AUC Tumor Alone))×100%.

Proliferation Assay: Anti-DNP KIR-CAR engineered T cells (1E6 cells) labelled with CellTrace Violet dye (Cat No. C34557 Invitrogen) were incubated with uPAR rPIM, uPAR SuFEX PIM, uPAR Ph (N) PIM, and A172 tumor targets at an effector:target ratio of 2:1, or left unstimulated in media. All proliferation assay samples were incubated for 4 days at 37° C. and 5% CO₂. Cells were then stained with Live/Dead Fixable Near-IR stain (Cat No. L10119, Invitrogen), PerCP-Cy5.5-conjugated mouse anti-human CD8a (Cat No. 45-0088-42, eBioscience), Alexa Fluor 700-conjugated mouse anti-human CD4 (Cat No. 56-0048-82, eBioscience), and BV605-conjugated mouse anti-human CD3 (Cat No. 300460 BioLegend). Flow cytometry data were acquired on a Cytoflex S flow cytometer. Results were analysed with FCS Express (De Novo Software) by determining the starting generation peak based on the unstimulated sample and using the software proliferation package for fitting a proliferation model and collecting corresponding statistics, such as percent divided. FlowJo analysis software was used generate histogram CTV dilution curves.

Antibody-dependent cellular phagocytosis (ADCP) assay: For preparation of effector monocytes, U937 monocytes were seeded at 500,000 cells/mL and activated with IFN-γ (0.1 mg/mL) 24 hours prior to the phagocytosis assay. The IFNg-treated monocytes were then counted and washed thrice in neat Roswell Park Memorial Institute (RPMI) 1640 medium. The monocytes were suspended to a concentration of 1 million cells/mL and stained with 1.9 μM Vybrant™ DiD Cell-Labelling Solution for 30 minutes (37° C., 5% CO₂). Cells were then washed thrice with warm assay media (14% Ultra Low IgG FBS in RPMI) and resuspended to a concentration of 6.0 million cells/mL to be plated for use in assay. On the day of the experiment, A172 target cells were collected by incubation in 1 mM ethylenediaminetetraacetic acid (EDTA) in 1X phosphate buffered saline (PBS) followed by quenching with complete growth media. These A172 cells were counted and washed thrice with neat RPMI. A172 cells were then suspended to a concentration of 1 million cells/mL and stained with 5.7 μM Vybrant DiO Cell-Labelling Solution for 30 minutes (37° C., 5% CO₂). The labelled A172 cells were then washed thrice with warm assay media (14% Ultra Low IgG FBS in RPMI) and resuspended to a concentration of 3.0 million cells/mL to be plated for use in the phagocytosis assay. rPIM:Ab complexes were incubated for 2 hours prior to addition to monocyte/A172 co-cultures by combining 200 nM rPIM and 100 nM of SPE7 mAb in 25 μL of RPMI. The pre-complexed rPIM/SPE7 solution was added to the phagocytosis to yield a final concentration of 50 nM rPIM:25 nM mAb during phagocytosis. All samples were run in duplicate. To a U-bottom 96-well plate, 50 μL of A172 cells were incubated with 25 μL of antibody:rPIM incubation for 10 minutes. Next, 25 μL of the monocyte suspension was added. The plate was centrifuged at 800 rpm for 2 minutes to pellet cells and placed in an incubator (37° C., 5% CO₂) for 1 hour. Plates were placed on ice and all samples were collected and analyzed on a Cytoflex LX flow cytometer. Gains were APC-Cy 7: 30. AF488: 6. FSC: 81 SSC: 109. ADCP percentage was calculated by dividing double positive events by the limiting quadrant cell number (Q2/Q2+Q1).

### Example 3. RPIMs Allow for Increased Function and Display Prolonged Bioavailability

### Results

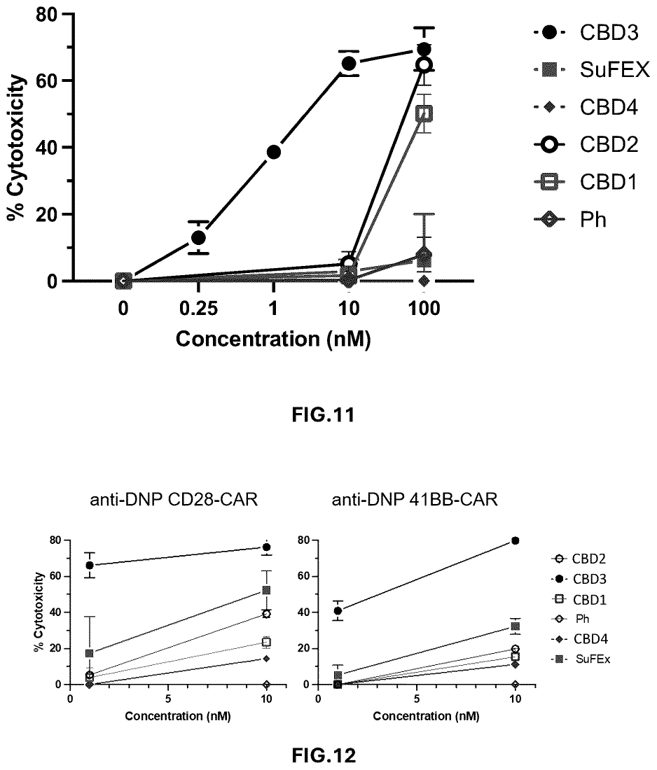

To fully test to what extent reversibility is important, and further understand the interplay between kinetics and function, the PIMs were tested in other environments. It was observed that reversibility and tuning of rPIMs with different electrophiles can allow for increased function depending on target. K562 tumor cells were engineered to express uPAR fused to mCherry for fluorescent imaging. These were then co-cultured with anti-DNP KIR-CAR T cells and a range of concentrations of rPIMs (compounds I-1, 1-2, 1-3 or 1-4), SuFEX PIM, and Ph (N) PIM. Cytotoxicity was assessed over a 4-day live cell imaging assay. Results demonstrated that CBD3, CBD2, and CBD1 retain the ability to elicit cytotoxicity against K562 cells whereas the CBD4 and SuFEX, with low to no dissociation, cannot elicit cytotoxicity. As seen previously, the non-covalent Ph adapter also does not elicit any cytotoxicity showing that killing of K562 cells was dependent upon reversible covalent binding as DNP KIR-CAR T cells were unable to kill K562 cells when co-cultured in the presence of the non-reversible SuFEX PIM or the (N)PIM, Ph (FIG. 11). The rPIMs (CBD3, CBD2, and CBD1) elicited varying degrees of killing, with the CBD3 molecules being the most potent once again. It was previously observed that T cells engineered with chimeric antigen receptors specific for DNP (DNP CAR T cells) had less reliance on covalency for function when compared to non-covalent molecules. Therefore, the ability of the rPIMs to direct CAR T cell cytotoxicity against A172 using two prototypic, second-generation CARs was tested (one containing a CD28 costimulatory domain and the other containing a CD137 costimulatory domain) (FIG. 8B). T cells were engineered with second-generation CARs that include either the CD28 or CD137 costimulatory domains. Both CARs employ an scFv against DNP to serve as a binding partner for the PIMs. Anti-DNP CD28-CAR T cells and anti-DNP CD137-CAR T cells were co-cultured with uPAR expressing A172 tumor cells for 4 days with 1 nM or 10 nM uPAR CBD3 rPIM (compound 1-3), SuFEX PIM, or Ph (N) PIM. Here, again, the CBD3 rPIM elicited the greatest cytotoxicity and the same hierarchy of functionality and potency among the rPIMs, SuFEX PIM and Ph (N) PIM that was observed in the prior cytotoxicity assays, was observed here. This same trend seen with the DNP KIR-CAR T cells demonstrates that the hierarchy of rPIM functionality is consistent for other synthetic receptors, which further shows that reversibility with the CBD3 rPIM is a universally beneficial property (FIG. 12). The reversibility of the rPIM allows for the initial starting material to be reformed once unbound. This was tested by setting up a cytotoxicity assay and probing the supernatant at different periods to assess what level of functional molecule is still present. Cell cultures were set up with 50 nM uPAR CBD3 rPIM or uPAR SuFEX incubated in media alone (FIG. 13A), with DNP—KIR-CAR-T cells alone (FIG. 13B), or with DNP—KIR-CAR-T cells in the presence of A172 eGFP cells (FIG. 13C). After 24 and 72 hours of incubation, the supernatant was then separated, collected and used to functionalize fresh DNP—KIR-CAR T cells to assess activation elicited by PIMs still available in solution. Fresh CBD3 rPIM and SuFEX PIM was used as a positive control to normalize the data. To assess functionality, the fresh DNP—KIR-CAR T cells were co-cultured with A172 eGFP cells to provide a stimulus. Maximal stimulation by the uPAR CBD3 rPIM and uPAR SuFEX PIM was determined using a parallel culture at each time point where fresh DNP—KIR-CAR-T cells were cultured with fresh rPIM and PIM in the presence of A172 eGFP cells. The activity of the cultures using the spent supernatant was normalized to the activity of the cultures using fresh reagents. The CBD3 rPIM showed at both 24 and 72 hours that it retained >90% of its original function whereas the ability for SuFEX to facilitate activation drastically decreased (FIG. 13A-13C). The increased bioavailability of the CBD3 rPIM when cultured with T cells and tumor cells when compared to the irreversible covalent SuFEX PIM demonstrates that the reversible nature of these electrophiles does both limit hydrolysis of the material over time and allow for some level of regeneration of starting material after labelling.

### Methods

K562 uPAR-mCherry T Cell Cytotoxicity Assay: K562 cells were engineered with a uPAR-mCherry fusion protein. In these experiments 5E3 tumor cells per well were pre-plated in a 96 well flatbottom plate overnight. The next day, anti-DNP KIR-CAR ap T cells were added to the tumor cells at an effector to target ratio of 8:1. Tumor targeting rPIMs molecules were added to the T cell:tumor cell co-cultures at various concentrations (0-50 nM) and monitored for 4 days at 37° C. and 5% CO₂ in the Sartorius Incucyte S3 Live cell imaging system with 9 images per well taken every 8 hours. Red image mean for each image was used to determine tumor cell growth. The area under the growth curve (AUC) was analyzed using PRISM Graphpad and used as a metric for tumor cell growth for this data. The larger the area, the greater the tumor cell growth that occurred over the incubation period. The area under the curve for the tumor alone control and each condition were used to calculate the % cytotoxicity. Percent cytotoxicity was calculated as: % Cytotoxicity=((AUC Tumor Alone-AUC Sample)/(AUC Tumor Alone))×100%.

CD28 and CD137 CAR T Cell Cytotoxicity Assay: A172 cells glioblastoma cells were engineered to express eGFP lentivirus made. In these experiments 5×10³ tumor cells per well were pre-plated in a 96 well flatbottom plate overnight. The next day anti-DNP CD28 or CD137 CAR ap T cells were added to the tumor cells at an effector to target ratio of 8:1. Tumor targeting rPIMs molecules were added to the co-cultures at various concentrations at 1 nM or 10 nM and monitored for 4 days at 37° C. and 5% CO₂ in the Sartorius Incucyte S3 Live cell imaging system with 9 images per well taken every 8 hours. Green image mean for each image was used to determine tumor cell growth. The area under the growth curve (AUC) was analyzed using PRISM Graphpad and used as a metric for tumor cell growth for this data. The larger the area, the greater the tumor cell growth that occurred over the incubation period. The area under the curve for the tumor alone control and each condition were used to calculate the % cytotoxicity. Percent cytotoxicity was calculated as: % Cytotoxicity=((AUC Tumor Alone-AUC Sample)/(AUC Tumor Alone))×100%.

rPIM Bioavailability Assay: A172 cells glioblastoma cells were engineered to express eGFP lentivirus. In these experiments 5×10³ tumor cells per well were pre-plated in a 96 well flatbottom plate overnight. The next day anti-DNP KIR-CAR ap T cells were added to the tumor cells at an effector to target ratio of 8:1 followed by the uPAR CBD3 rPIM or uPAR SuFEX PIM at 50 nM. CBD3 and SuFEX PIMs were also added to media alone and anti-DNP KIR-CAR T cells without tumor cells. Assay conditions were set up in triplicates to allow for imaging and to take triplicate wells of each condition at each timepoint. The co-cultures were monitored for 3 days at 37° C. and 5% CO₂ in the Sartorius Incucyte S3 Live cell imaging system with 9 images per well taken every 8 hours. At 24 and 72 hours the contents of the wells were resuspended well, transferred to a 96 well u-bottom, and spun down to pellet cells and debris. 170 μL of supernatant was then taken and added to 4×10 anti-DNP KIR-CAR αβ T cells and 4*10⁵ A172 eGFP tumor cells in a 96 well flat bottom plate that were in 40 μL of media. As a control supernatant from the killing assay was also added to T cells alone to ensure the supernatant itself was not responsible for activation. Fresh CBD3 uPAR rPIM and uPAR SuFEX PIM was also used as a positive control for normalization. The activation assay was set up for 3 hours at 37° C. and 5% CO₂. Cells were then surface stained with Pacific Blue-conjugated mouse anti-human CD4 (Cat No. 558116, BD Pharmingen), AlexaFluor700-conjugated mouse anti-human CD8u (Cat No. 56-0086-82, Invitrogen), and anti-huCD69 (Cat No. 563835, BD Horizon). CD69 expression was analyzed by flow cytometry and normalized to the positive control.

### Example 4. RPIM Warheads Allow for Increased Function Across Multiple Targets, and Show Enhanced Function In-Vivo

### Results

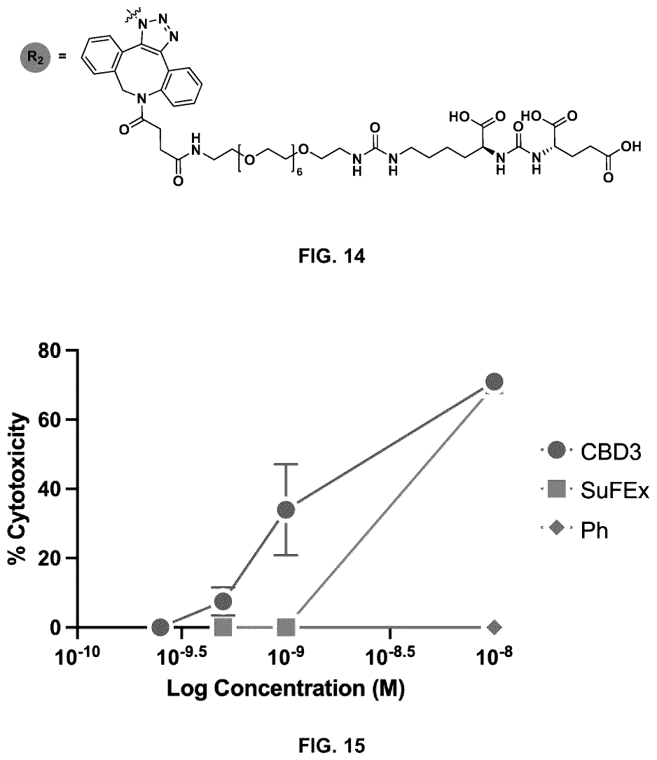

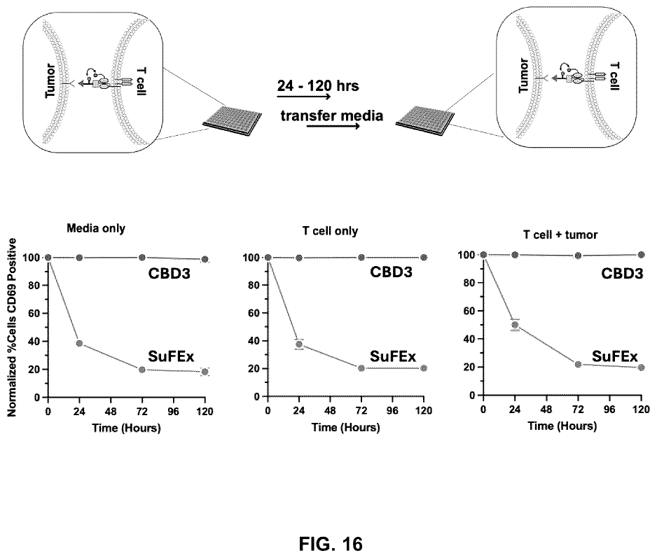

To understand the broad applicability of the use of reversible electrophiles for cell-cell proximity induction, ligands were incorporated to target other tumor antigens. First, the small molecule glutamate urea lysine (GUL) was incorporated into target prostate specific membrane antigen (PSMA) (FIG. 14). As the CBD3 electrophile was the most potent in previous studies, research only considered comparison to the irreversible covalent SuFEX electrophile and non-covalent Ph. When testing cytotoxic function of anti-DNP KIR-CAR T cells co-cultured with varying concentrations of these adapters, enhanced function with the GUL CBD3 rPIM (compound I-9) was observed, especially at low concentrations (FIG. 15). When then comparing stability in a bioavailability assay like what was done with the uPAR rPIM and PIM, again enhanced adapter bioavailability with the CBD3 rPIM (FIG. 16) was observed. Anti-DNP KIR-CAR T cells, LnCap tumor cells, and 100 nM of GUL CBD3 rPIM or GUL SuFEX PIM were co-cultured for 24, 72, and 120 hours. The supernatant was then separated from the cells and cultured with fresh T cells and K562 uPAR-mCherry cells to assess activation elicited by adapter still available in solution. Fresh CBD3 rPIM and SuFEX PIM was used as a positive control to normalize the data. At all time points the CBD3 rPIM retained in solution was able to elicit ~100% of original function whereas ability for SuFEX to facilitate activation diminished rapidly. At 24, 72, and 120 hours, the CBD3 rPIM is able to elicit full function, regardless of if the adapter was previously incubated in media alone, in T cells alone, or in a killing assay setup. In comparison the irreversible SuFEx adapter showed rapid loss of function regardless of which condition it was exposed to prior to the activation readout assay.

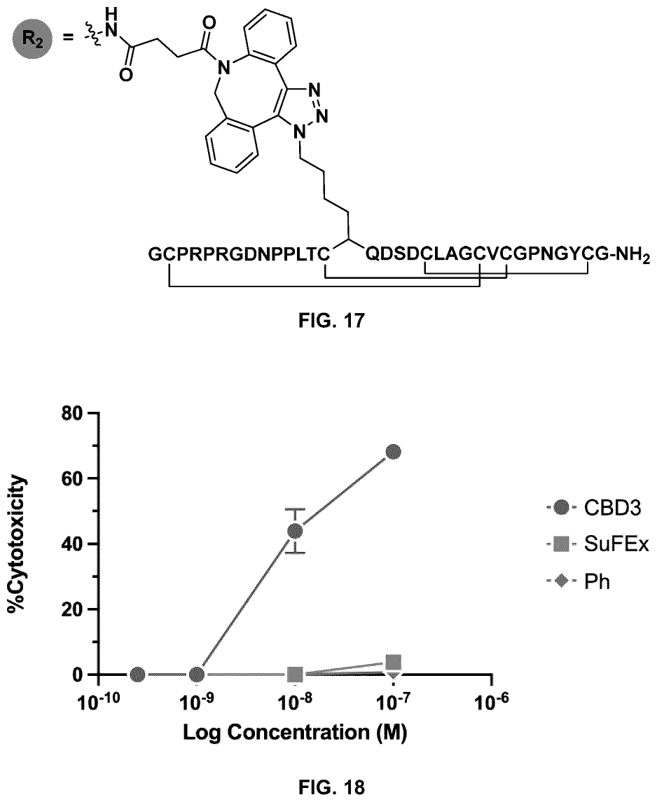

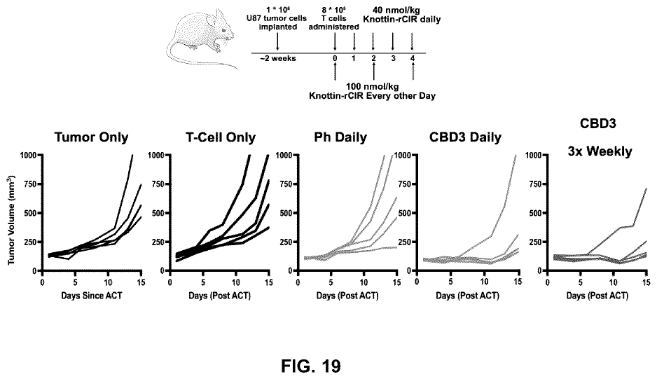

As a larger targeting ligand, the integrin binding “knottin” RGD peptide (FIG. 17) was incorporated. Assessing the cytotoxicity of the anti-DNP KIR-CAR T cells against U87-MG tumor cells when cultured with varying concentrations (0.5 nM-100 nM) of the knottin CBD3 rPIM (compound I-10), knottin SuFEx PIM, and knottin Ph NPIM revealed the necessity for the reversible electrophile in these adapters to be able to elicit cytotoxicity at any concentration (FIG. 18). To assess utility in a xenograft mouse model, mice were inoculated with the U87-MG tumor cells and then treated with anti-DNP KIR-CAR T Cells, the knottin Ph NPIM daily at 40 nmol/kg, and the knottin CBD3 rPIM (also referred to as Knottin rCIR) either daily at 40 nmol/kg or 3x weekly at 100 nmol/kg (FIG. 19).Treatment with the knottin CBD3 adapter either daily or 3x weekly at a higher concentration both resulted in enhanced control of tumor growth compared to the Ph NPIM, or T cells alone, both which showed no overall impact on tumor volume. It was observed that dosing with the Ph NPIM displayed little to no increase in the ability for the T cells to slow tumor growth compared to the T cell only treatment group. The CBD3 rPIM however in both treatment protocols did show enhanced tumor control over 10-12 days, with the tumors eventually beginning to grow out, displaying the benefit of the reversible electrophile extends in-vivo. Overall, the advantages of using reversible electrophiles for cell-cell proximity induction can be observed across a range of tumor targeting ligands, both in-vitro and in-vivo, displaying the broad applicability.

### Methods

Live Cell Imaging Cytotoxicity Assay: U87-MG integrin expressing tumor cells were engineered with eGFP lentivirus made in house. LnCAP cells were engineered with Nuclight™ Red lentivirus (Sartorius, Cat No. 476). In these experiments 5E3 tumor cells per well were pre plated in a 96 well flatbottom plate overnight. The next day anti-DNP KIR-CAR ap T cells were added to the tumor cells at an effector to target ratio of 8:1. Also added to the wells were the appropriate tumor targeting rPIMs molecules at various concentrations (0-100 nM). The 3 components were co-cultured for 4-5 days at 37° C. and 5% CO₂ in the Sartorius Incucyte™ S3 Live cell imaging system with 9 images per well taken every 8 hours. Green or red image mean for each image was used to determine tumor cell growth. The area under the growth curve (AUC) was analyzed using PRISM Graphpad and used as a metric for tumor cell growth for this data. The larger the area, the greater the tumor cell growth that occurred over the incubation period. The area under the curve for the tumor alone control and each condition were used to calculate the % cytotoxicity.

Percent cytotoxicity was calculated as:

$$\% Cytotoxicity = ( ( AUC Tumor Alone - AUC Sample ) / ( AUC Tumor Alone ) ) \times 100 \%$$

rPIMBioavailabilityAssay: PSMA positive LnCap prostate cancer cells were engineered to express Nuclight Red using lentivirus from Sartorius. In these experiments 5E3 tumor cells per well were pre plated in a 96 well flatbottom plate overnight. The next day anti-DNP KIR-CAR ap T cells were added to the tumor cells at an effector to target ratio of 8:1 followed by the GUL-CBD3 rPIM or GUL-SuFEX PIM at 50 nM. CBD3 and SuFEX adapters were also added to media alone and anti-DNP KIR-CAR T cells without tumor cells. Assay conditions were set up in 9 wells to allow for imaging and to take triplicate wells of each condition at each timepoint. The co-cultures were monitored for 5 days at 37° C. and 5% CO₂ in the Sartorius Incucyte S3 Live cell imaging system with 9 images per well taken every 8 hours. At 24, 72, and 120 hours the contents of the wells were resuspended well, transferred to a 96 well u-bottom, and spun down to pellet cells and debris. 170 L of supernatant was then taken and added to 4E5 anti-DNP KIR-CAR ap T cells and 4E5 LnCap nuclight Red tumor cells in a 96 well flat bottom plate that were in 40 μL of media. As a control, supernatant from the killing assay was also added to T cells alone to ensure the supernatant itself was not responsible for activation. Fresh CBD3 GUL-rPIM and GUL-SuFEX PIM was also used as a positive control for normalization. The activation assay was set up for 3 hours at 37° C. and 5% CO₂. Cells were then surface stained with Pacific Blue-conjugated mouse anti-human CD4 (Cat No. 558116, BD Pharmingen), AlexaFluor700-conjugated mouse anti-human CD8a (Cat No. 56-0086-82, Invitrogen), and anti-huCD69 (Cat No. 563835, BD Horizon). CD69 expression was analyzed by flow cytometry and normalized to the positive control.

Xenograft Mouse Models: NRG Mice were implanted with 1E6 U87-MG tumor cells in 50 μL of a 1:1 mixture of PBS to Matrigel (Gibco). Tumors were grown for 14-16 days until they grew to a treatable size of 100 mm³. 8E6 engineered anti-DNP KIR-CAR T cells were injected into the mice via tail vein injection in 200 μL. Mice were then dosed either intraperitoneal or subcutaneous with the adapters depending on the study. Mice were dosed with either 40nmol/kg or 100nmol/kg either daily or every other day depending on the study. Tumors were measured with calipers until they reached a maximum size of 2000 mm³.

### Example 5: Characterizing the Thermodynamic and Kinetic Landscape of Reversible Covalent Electrophiles

The dual reversible covalency in a model system was explored, where anti-DNP antibodies or synthetic T cell receptors are the immune-side target and uPAR is the tumor-side target. As described above, CBD3 was identified as an effective reversible electrophile for developing monocovalent universal T cell adaptors. Here, the kinetic and thermodynamic properties of various lysine-targeting electrophiles are investigated to help develop dual covalent approaches.

Previously described anti-DNP targeting reversible covalent small molecules that feature 2-APBA (CBD3), salicylaldehyde (2-hydroxybenzaldehyde) (CBD2), and benzaldehyde (CBD1) reversible electrophiles, along with phenyl (Ph) as a non-covalent control and SuFEx as an irreversible control were utilized. First, a reductive trapping approach followed by SDS-PAGE was considered to measure the residence time of each reversible covalent immune recruiter (rPIM) (FIG. 4B). Each rPIM was pre-incubated with anti-DNP monoclonal antibody (SPE7), followed by spiking with DNP-glycine competitor for various time points. In this format, CBD1 showed the highest residence time, with some complex remaining after 5 mins of incubation with competitor. CBD3 and CBD2 showed covalent engagement of SPE7, and completely dissociated after 5 mins of incubation.

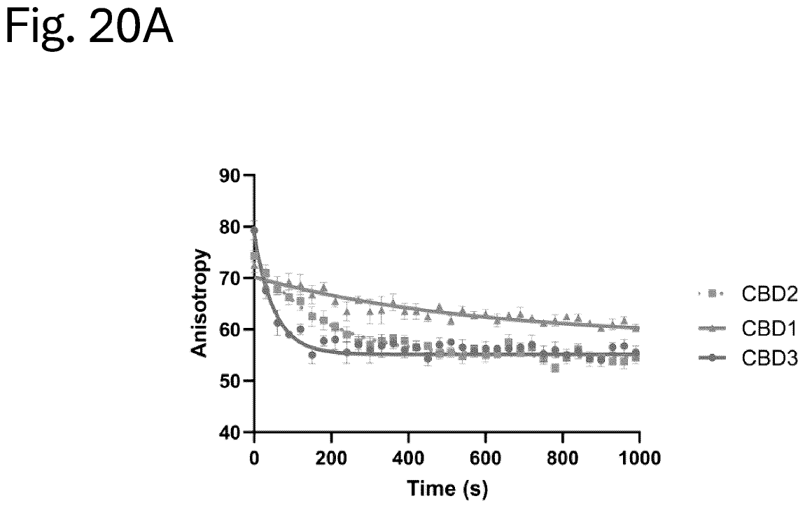

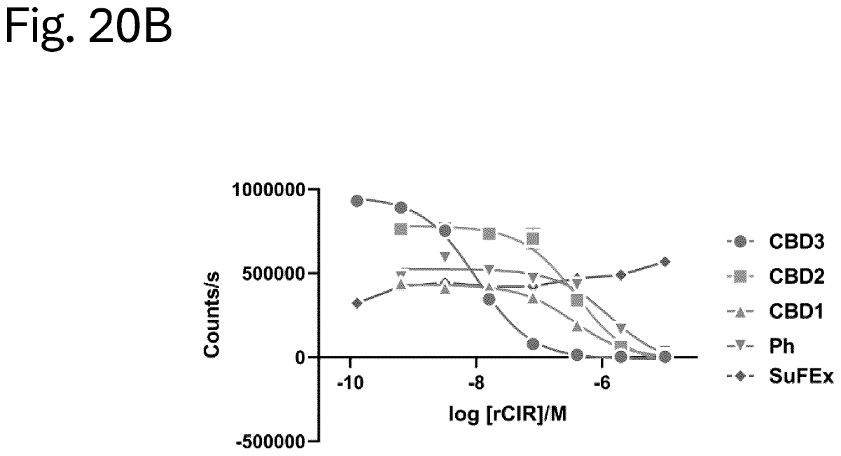

To more quantitatively measure the residence time of each reversible covalent electrophile, a fluorescence polarization (FP) assay was conducted (experimental details provided below). Each fluorescently tagged rPIM was incubated at saturation with SPE7, followed by spiking with excess DNP-glycine. Dissociation was monitored by reading fluorescence polarization over time (FIG. 20A, Table 1). Interestingly, CBD3 showed the fastest dissociation half-life, indicative of a low kinetic barrier to imine formation.

TABLE 1
Dissociation rates determined for competitor-
induced disruption of rPIM-SPE7 interactions.

| rPIM | k_{off} | t_{1/2} |
|---|---|---|
| CBD3 | 1.9 * 10⁻² ± 0.3 * 10⁻² s⁻¹ | 36 ± 5 s |
| CBD2 | 5.7 * 10⁻³ ± 0.5 * 10⁻³ s⁻¹ | 120 ± 10 s |
| CBD1 | 1.8 * 10⁻³ ± 0.3 * 10⁻³ s⁻¹ | 390 ± 60 s |

With knowledge of the kinetic barriers to imine formation in hand, the thermodynamic stability provided by each reversible electrophile was investigated. AlphaLISA (experimental details provided below) was used in a competition format to measure the binary binding affinity of each rPIM (FIG. 20B, Table 2).

TABLE 2
AlphaLISA competition-derived binding
affinities of rPIM-SPE7 interaction.

| rPIM | K_{d} | |
|---|---|---|
| CBD3 | 10.0 ± 0.6 | nM |
| CBD2 | 390 ± 60 | nM |
| CBD1 | 350 ± 50 | nM |
| Ph | 1 300 ± 400 | nM |

| | |
|---|---|
| SuFEx | Not determined - no |
| | competition |

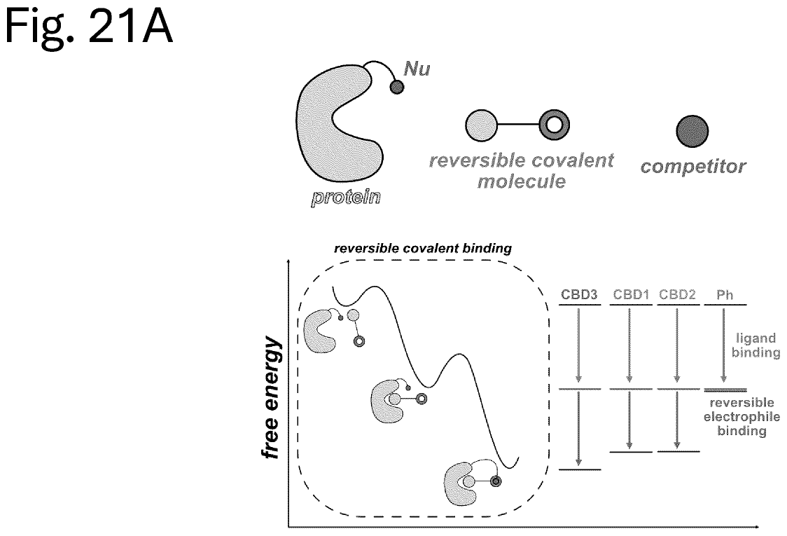

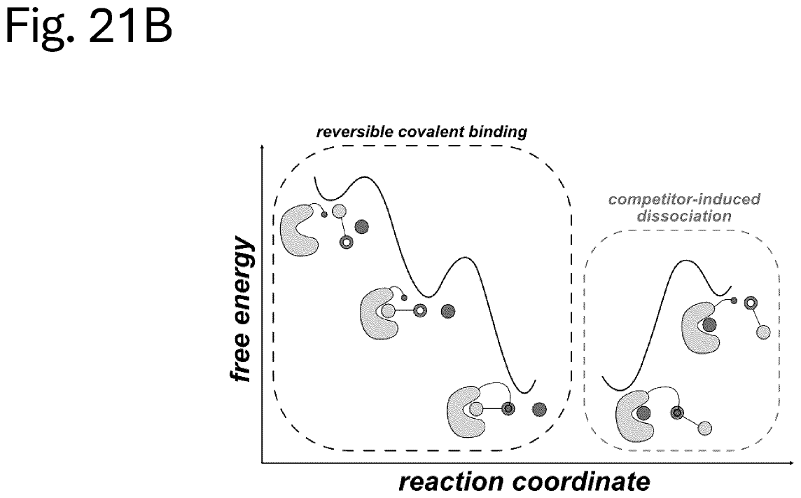

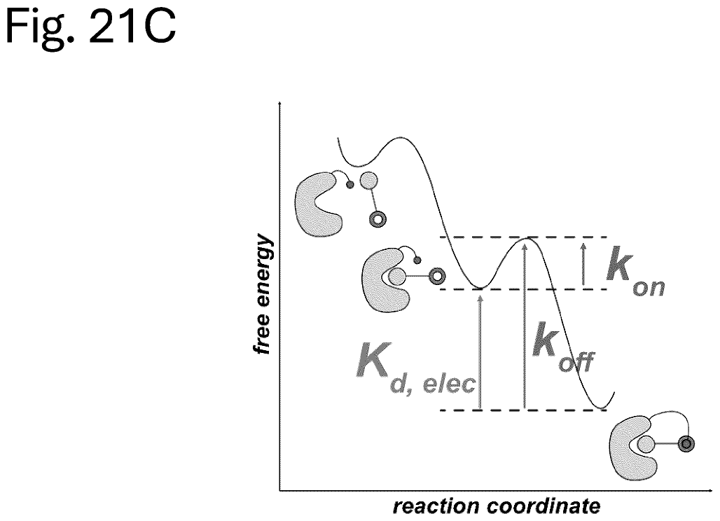

Based on the kinetic and thermodynamic parameters of imine formation determined in the previous experiments, a model of reversible covalent binding based on a two-step reaction process was constructed where the covalent reaction is preceded by a reversible, non-covalent binding interaction (FIG. 21A).The binding energy associated with the reversible covalent step can be isolated by subtracting the binding energy of the Ph non-covalent control (Table 3). Here, CBD3 provides the greatest additional thermodynamic stability.

TABLE 3
Binding affinities and free energies of reversible
electrophile-lysine interactions.

| rPIM | K_{d, electrophile} | −ΔG_{electrophile} | |
|---|---|---|---|
| CBD3 | 7 ± 2 mM | 2.9 ± 0.2 | kcal mol⁻¹ |
| CBD2 | 290 ± 90 mM | 0.7 ± 0.2 | kcal mol⁻¹ |
| CBD1 | 260 ± 80 mM | 0.8 ± 0.2 | kcal mol⁻¹ |
| Ph | — | 0 | kcal mol⁻¹ |

The dissociation rates in the presence of competitor provide the intrinsic hydrolysis rate of the imine, or k_{off}, (FIG. 21B), which together with the thermodynamic binding affinities allow us to calculate the kₒₙ for each electrophile (FIG. 21C, Table 4).

The dissociation rates in the presence of competitor provide the intrinsic hydrolysis rate of the imine, or k_{off}, (FIG. 21B), which together with the thermodynamic binding affinities allow us to calculate the kₒₙ for each electrophile (FIG. 21C, Table 4).

TABLE 4
On-rates of reversible electrophile-lysine reactions.

| rPIM | kₒₙ | |
|---|---|---|
| CBD3 | 2.6 ± 0.8 | M⁻¹ s⁻¹ |
| CBD2 | 2.0 * 10⁻² ± 0.7 * 10⁻² | M⁻¹ s⁻¹ |
| CBD1 | 7 * 10⁻³ ± 2 * 10⁻³ | M⁻¹ s⁻¹ |

This parameter determines how fast each electrophile can form covalent complex, which is of importance in cell-cell proximity stabilization contexts where covalency may be required for synapse stability and downstream function. Here, CBD3 has the fastest kₒₙ by 2 orders of magnitude. CBD3's low kinetic barrier to imine formation allows rapid and dynamic covalent engagement, and its high thermodynamic stability allows for potent target engagement at low nanomolar concentrations of small molecule. These properties make it an ideal candidate for building dual reversible covalent small molecules.

### Example 6: Building Dual Reversible Covalent Molecular Glue Mimics

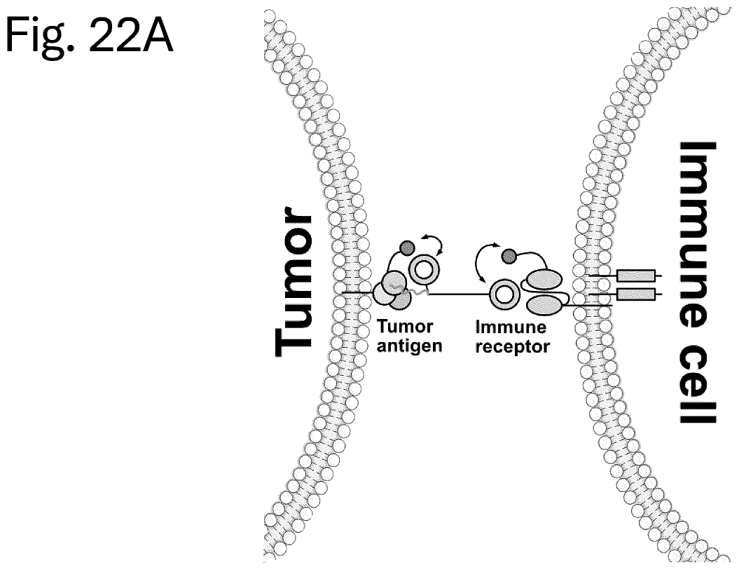

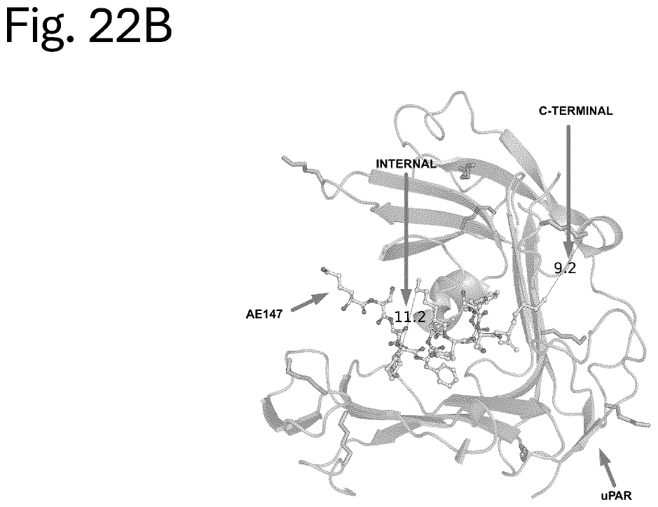

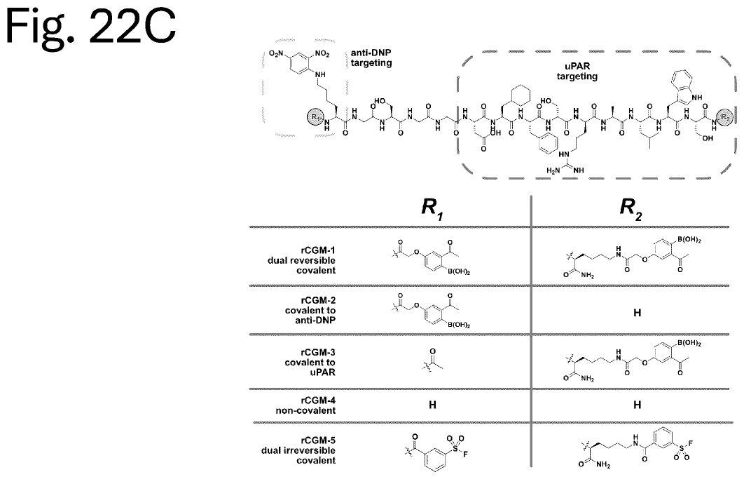

CBD3's unique kinetic and thermodynamic properties identify it as the best candidate for a dual reversible PIM (also referred to as a reversible covalent glue mimic or rCGM herein) that bridges immune cells and tumor cells (FIG. 22A). However, the optimal electrophile placement for targeting the tumor antigen, uPAR, remained unknown. In previous work on irreversible CGMs, sulfonyl fluoride electrophiles were placed at both “internal” and “C-terminal” positions in the uPAR-binding peptide sequence.³ These positions were chosen as they are amenable to substitution without drastically affecting affinity. Sulfonyl fluorides are “residue-agnostic” electrophiles, as they react with tyrosine, lysine, and histidine residues.⁴ CBD3, in contrast, reacts exclusively with lysine. To ensure that lysine residues were present in the vicinity of the uPAR-peptide binding site, consideration was given to a published crystal structure of uPAR bound to the peptide AE147, a close analog of AE133 (FIG. 22B).⁵ It was found that both C-terminal and internal positions on the peptide were within 15 Å of lysine residues on uPAR, making both positions possible candidates for reversible covalent uPAR binders. The C-terminal position was pursued further, as it is slightly closer to a potential target lysine and farther away from the N-terminal end of the peptide and the anti-DNP-targeting region. This reduces the likelihood of undesirable in cis electrophilic engagement of anti-DNP.

A C-terminal, dual 2-APBA-functionalized rCGM (rCGM-1, compound I-11) was synthesized. In addition, two control single rCGM (rPIM) molecules: i) a covalent-to-DNP peptide lacking covalency towards uPAR (rCGM-2, compound 1-12), and ii) a covalent-to-uPAR peptide lacking covalency towards anti-DNP (rCGM-3, compound 1-13) were synthesized (FIG. 22C). Non-covalent (rCGM-4) and dual irreversible covalent (rCGM-5) peptides were prepared as previously described.³

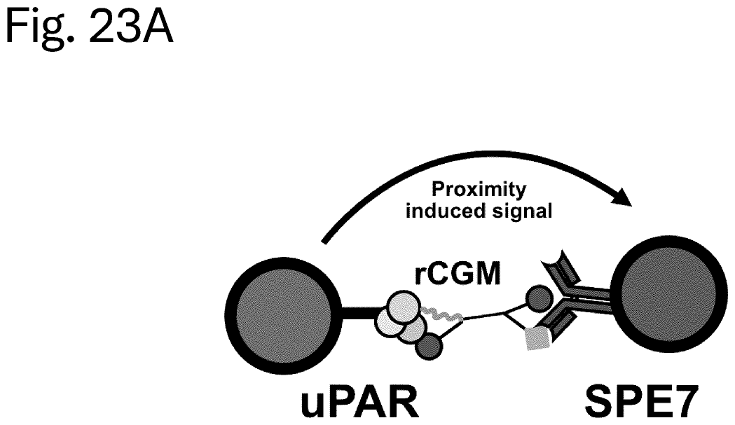

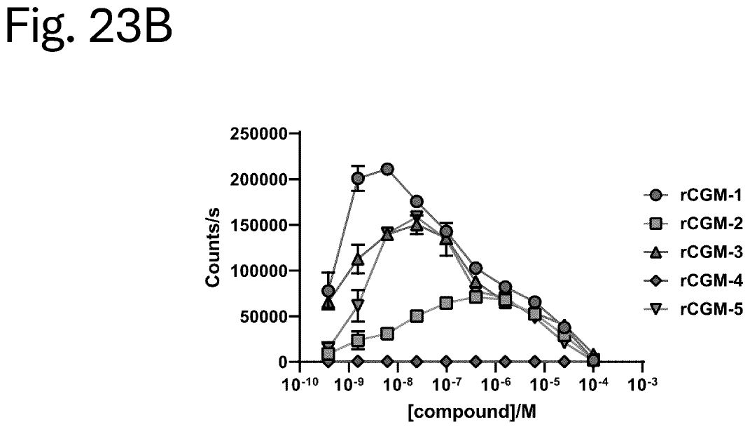

AlphaLISA was then considered to assess the ternary complex formation induced by rCGMs and controls (FIG. 23A, FIG. 23B). A large increase in ternary complex potency for rCGM-1 (compound I-11) in comparison to the other compounds was observed. rCGM-1 (compound I-11) forms more ternary complex (higher peak) and is also more potent (left-shifted binding curve). rCGM-4, the non-covalent control, completely failed to form noticeable ternary complex, highlighting the difficulty of building potent “cell-cell” proximity inducing molecules. rCGM-5, the irreversible control, showed similar potency to rCGM-3 (compound I-13). All compounds still exhibit the characteristic auto-inhibition expected with bifunctionals in non-cooperative or negatively cooperative systems. rCGM-3 (compound I-13), which is covalent towards uPAR, is more potent than rCGM-2 (compound I-12), which is covalent towards SPE7.

Next the efficacy of rCGMs in inducing T cell killing was tested (FIG. 24A). DAP12 synthetic antigen receptor T cells were used with an SPE7-derived scFv. Target cells were GFP-expressing A172s, which have upregulated surface uPAR. T cell killing was assessed by live-cell imaging (FIG. 24B). rCGM-1 (compound I-11) and rCGM-3 (compound I-13) showed similar potencies. rCGM-5 showed reduced potency, likely due to hydrolytic and off-target deactivation over the course of the assay. rCGM-2 (compound I-12) and rCGM-4 showed no efficacy over the concentration range tested. rCGM-2 (compound I-12) is less efficacious than rCGM-3 (compound I-13), despite them both being singly covalent adaptors.

These results indicate that the uPAR-bifunctional interaction may be the limiting component of the ternary complex interaction, and stabilizing this “weakest link” provides greater functional benefits than enhancing the stability of the SPE7-bifunctional interaction.

Dual reversible CGM bridging target cells and effector cells induce antibody dependent cellular phagocytosis (FIG. 25A). Two-color flow-cytometry was utilized to examine such interaction (FIG. 25B). Various concentrations of rCGM-1 (compound I-11), rCGM-2 (compound I-12), and rCGM-4 were incubated with ES-2 target cells for 30 min prior to addition of THP-1 monocytes for 1 hr. ES-2 cells were detached using 0.5 mM EDTA in PBS for 5 min at 37° C. Cells were collected and counted to confirm viability and washed three times in serum-free assay medium (AM; neat RPMI 1640). Following washing, cells were pelleted and resuspended in AM at 1.0×10\hat{ }6 cells mL⁻¹. Target cells were labelled by adding Vybrant™ DiO (Thermo Fisher Scientific; 1 mM) to a final concentration of 5.7 μM and incubating for 30 min at 37° C. Labelled cells were washed three times with AM and resuspended at 6.0×10\hat{ }6 cells mL⁻¹. For assays, 150,000 target cells were added per well in 25 μL. THP-1 monocytes were counted to assess viability and washed three times with serum-free AM. Cells were pelleted and resuspended in neat RPMI at 1.0×10\hat{ }6 cells mL⁻¹ and labelled with Vybrant DiD (Thermo Fisher Scientific, 1 mM) at a final concentration of 1.9 μM for 30 min at 37° C. Labelled cells were washed three times with AM and resuspended at 3.0×10\hat{ }6 cells mL⁻¹. For co-culture, 150,000 monocytes were added per well in 50 μL. ES-2 target cells were then combined with 25 μL of rCGM-1 (compound I-11), rCGM-2 (compound 1-12), or rCGM-4 and incubated for 30 min at 37° C. Subsequently, 50 μL of labelled THP-1 cells were added to designated wells; control wells received AM alone. Plates were centrifuged at 800×g for 2 min and incubated for 1 h at 37° C. to permit phagocytosis. Reactions were terminated by placing plates on ice prior to analysis. Samples were analyzed via two-color flow cytometry.

Dual covalent bifunctionals can leverage the added stability provided by covalency to induce ternary complexes in negatively cooperative settings. These “glue mimics” can achieve molecular glue-like kinetic stability without relying on serendipitous discovery or extensive medicinal chemistry optimization efforts. However, the irreversible electrophiles used in CGMs face off-target reactivity, hydrolysis, and slow reaction kinetics. These issues are compounded in the dual covalent setting, where in cis reactivity is undesirable. Reversible covalent electrophiles, with their hydrolytic stability and ability to “correct” off-target labelling, offer even more potent dual covalent proximity inducing molecules. rCGMs are developed as dual reversible covalent bi-functionals, and show that rCGMs induce higher levels of ternary complex in comparison to all other modalities. rCGMs also show functional enhancements in phagocytosis and T cell killing. This work demonstrates that dual reversible covalent bifunctionals display enhancements in ternary complex induction.

### Supplementary Methods

AlphaLISA Binary Complex Assay: rPIM-biotin (i.e. compound ID comprising Ph (control), compound 1-5 comprising CBD1, compound 1-6 comprising CBD2, compound I-7 comprising CBD3 and compound 6-D comprising SuFEx (control)) (20 μM) was combined with SPE7 (1 μM) and incubated overnight in 1X immunoassay buffer (Revvity, part no. AL000C). Incubations were then diluted to 2000X (to 10 nM of rPIM-biotin) and combined with 20 μg/mL of streptavidin donor (Revvity, part no. 67600025) and anti-Human IgG acceptor beads (Revvity, part no. AL103C). Corresponding rPIM-azide was then added at a range of concentrations to outcompete bead-bound rPIM-mAb interactions. The solutions incubated in the dark for 1 hr. The incubations were transferred to a 384-well AlphaPlate (Revvity, part no. 6008350) and read on a TECAN Spark plate reader with an Alpha Technology add-on. Default wavelengths, excitations, and integration times were used.

AlphaLISA Ternary Complex Assay: Varying concentrations of rCGM were incubated with 5 nM of recombinant uPAR-biotin and 5 nM of SPE7 mAb for 1 hr in 1X immunoassay buffer (Revvity, part no. ALOOOC) to allow for ternary complex formation. 10 μg/mL of AlphaLISA Anti-Human IgG (Fc specific) Acceptor Beads (Revvity, part no. AL103C) were added and the solutions incubated in the dark for 1 hr. Next, 10 μg/mL of AlphaScreen Streptavidin Donor Beads (Revvity, part no. 6760002S) were added and the solutions incubated in the dark for 1 hr. The incubations were transferred to a 384-well AlphaPlate (Revvity, part no. 6008350) and read on a TECAN Spark plate reader with an Alpha Technology add-on. Default wavelengths, excitations, and integration times were used.

SDS-PAGE: rCGMs (1 μM) were incubated with anti-DNP mAb (500 nM) and uPAR (1 μM) for 16 hours at room temperature. Additional conditions included 200 μM DNP glycine as a competitor, added 1 hr after rCGM-protein incubation. Sodium borohydride (5 mM) was added for 1 hr to reductively trap imine linkages. The samples were diluted in 2X Laemmli buffer and run on a polyacrylamide gel for 2 hrs at 120 V. Gels were stained with Coomassie Blue and imaged on a Odyssey imager.

Fluorescence Polarization Assay: Each rPIM alkyne (i.e. compound 2B comprising CBD1, compound 3B comprising CBD2 and compound 4B comprising CBD3) was clicked to an azide-AF488 fluorophore to make a fluorescent probe (rPIM-fluor). rPIM-fluor (20 nM) was incubated overnight with SPE7 (500 nM). Free DNP-glycine (20 μM) was then spiked in and fluorescence polarization read over time with a Tecan Spark plate reader.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### CITATIONS

1. Serniuck, N.J., Kapcan, E., Moogk, D., Moore, A.E., Lake, B.P.M., Denisova, G., Hammill, J.A., Bramson, J. L., and Rullo, A.F. (2024). Electrophilic proximity-inducing synthetic adapters enhance universal T cell function by covalently enforcing immune receptor signaling. Mol Ther Oncol 32, 200842. 10.1016/j.omton.2024.200842.
2. Krygier, K, Wijetunge, A.N., Srayeddin, A., Mccann, H., and Rullo, A.F. (2024). Leveraging Covalency to Stabilize Ternary Complex Formation For Cell-Cell Induced Proximity. ACS Chemical Biology, Vol 19/Issue 10. 10.1021/acschembio.4c00286
3. Kapcan, E.; Krygier, K.; da Luz, M.; Serniuck, N. J.; Zhang, A.; Bramson, J.; Rullo, A. F. Mimicry of Molecular Glues Using Dual Covalent Chimeras. Nat Commun 2025, 16 (1), 2855. https://doi.org/10.1038/s41467-025-58083-z.
4. Gilbert, K. E.; Vuorinen, A.; Aatkar, A.; Pog6ny, P.; Pettinger, J.; Grant, E. K.; Kirkpatrick, J. M.; Rittinger, K.; House, D.; Burley, G. A.; Bush, J. T. Profiling Sulfur(VI) Fluorides as Reactive Function-alities for Chemical Biology Tools and Expansion of the Ligandable Proteome. ACS Chem. Biol. 2023, 18 (2), 285-295. https://doi.org/10.1021/acschembio.2c00633.
5. Llinas, P.; Helene Le Du, M.; Gardsvoll, H.; Dano, K.; Ploug, M.; Gilquin, B.; Stura, E. A.; Menez, A. Crystal Structure of the Human Urokinase Plasminogen Activator Receptor Bound to an Antago-nist Peptide. The EMBO Journal 2005, 24 (9), 1655-1663. https://doi.org/10.1038/sj.emboj.7600635.
6. Grimster, N. P. Covalent PROTACs: The Best of Both Worlds?RSC Medicinal Chemistry 2021, 12 (9), 1452-1458. https://doi.org/10.1039/D1MD00191D.

1. A compound of Formula (I), Formula (II) or Formula (III) or a pharmaceutically acceptable salt and/or solvate thereof, 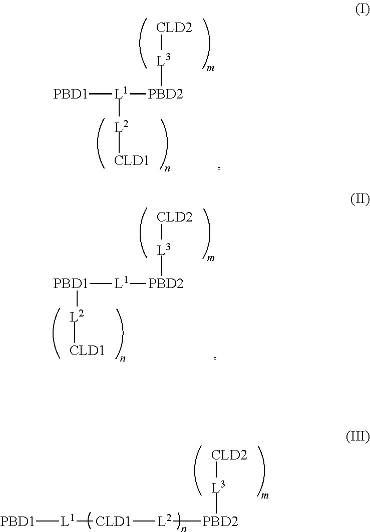
wherein PBD1 and PBD2 are each independently a protein binding domain (PBD) and CLD1 and CLD2 are each independently a covalent labeling domain (CLD), and L¹, L² and L³ are each independently a linker group;
n=0 or 1 and m=0 or 1, wherein at least one of n and m is 1,
wherein PBD1 selectively binds to a first target protein and PBD2 selectively binds to a second target protein, and wherein CLD1, if present, comprises a functional group that, upon binding of PBD1 to the first target protein, forms a reversible covalent bond with a nucleophilic group in the first target protein and CLD2, if present, comprises a functional group that, upon binding of PBD2 to the second target protein, forms a reversible covalent bond with a nucleophilic group in the second target protein.

2. The compound of claim 1, wherein CLD1 and CLD2 each independently comprise an electrophilic functional group that reacts with a nucleophilic moiety on the first and second target proteins.

3. The compound of claim 2, wherein the electrophilic functional group reacting with the nucleophilic moiety results in a reversible condensation reaction, to form a covalent bond, and optionally the electrophilic functional group is a ketone reacting with an amine to form an imine.

4. The compound of claim 1, wherein CLD1 and CLD2 each independently comprise an aldehyde, a boronic acid, an amide, a nitrile and/or a ketone.

5. The compound of claim 1, wherein CLD1 and CLD2 are each independently selected from:

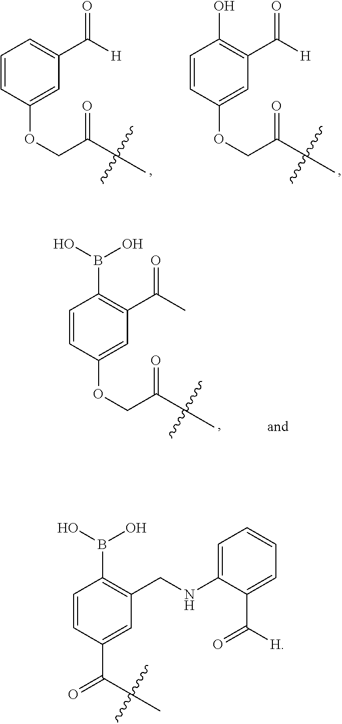

6. The compound of claim 1, wherein the first and second target proteins are each independently selected from proteins that are: cell surface receptors; overexpressed in a disease, disorder or condition; expressed on the surface of a cancer cell; and/or the first and second target proteins are each independently selected from a tumor antigen, an antibody and an immune cell receptor.

7. The compound of claim 1, wherein the first and second target proteins are each independently selected from urokinase plasminogen activating receptor (uPAR), prostate-specific membrane antigen (PSMA), human epidermal growth factor receptor 2 (HER2), an integrin, CD38, programmed death-ligand-1 (PD-L1), a G protein-coupled receptor (GPCR), a Kirsten rat sarcoma virus (KRAS), a vascular endothelial growth factor (VEGF) or folate receptor.

8. The compound of claim 1, wherein PBD1 and PBD2 are each independently selected from:
a) a di- or trinitrophenyl group having the following structure: 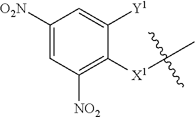
wherein Y¹ is H or NO₂;
X¹ is NR¹, O, CH₂, S(O), SO₂, SO₂O, OSO₂ or OSO₂O; and
R¹ is H, C₁₋₄alkyl or C(O)C₁₋₄alkyl;
b) a bicyclic nitro-substituted aromatic group having the following structure: 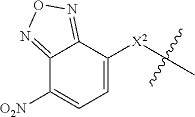
wherein X² is a bond, O, CH₂, NR² or S; and
R² is H, C₁₋₄alkyl or C(O)C₁₋₄alkyl;
c) a galactose-containing carbohydrate having the following structure: 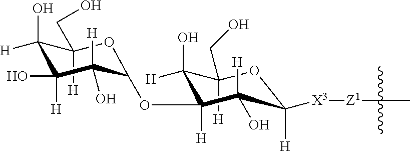
wherein X³ is CH₂, O, NR³ or S;
R³ is H or C₁₋₄alkyl; and
Z¹ is a bond, monosaccharide, disaccharide, oligosaccharide, glycoprotein or glycolipid;
d) a group having the following structure: 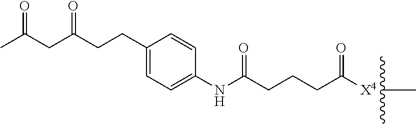
wherein X⁴ is O, CH₂ or NR⁴; and
R⁴ is H, C₁₋₄alkyl or C(O)C₁₋₄alkyl;
e) a group having the following structure: 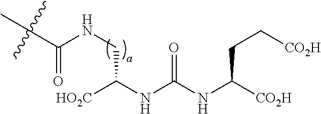
wherein a is an integer from 0 to 10, 1 to 15, 1 to 10, 1 to 8, or 1, 2, 3, 4, 5 or 6;
f) a group having the following structure: 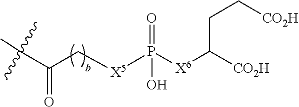
wherein X⁵ and X⁶ are independently CH₂, O, NH or S; and
b is an integer from 0 to 10, 1 to 15, 1 to 10, 1 to 8, or 1, 2, 3, 4, 5 or 6;
g) a group having the following structure: 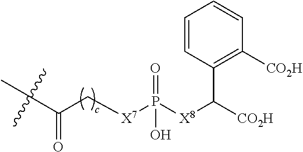
wherein X⁷ and X⁸ are independently CH₂, O, NH or S; and
c is an integer from 0 to 10, 1 to 15, 1 to 10, 1 to 8, or 1, 2, 3, 4, 5 or 6;
h) a group having the following structure: 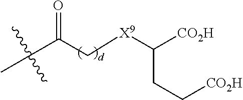
wherein X⁹ is O, CH₂, NR⁵, S(O), SO₂, SO₂O, OSO₂ or OSO₂O;
R⁵ is H, C₁₋₄alkyl or C(O)C₁₋₄alkyl; and
d is an integer from 0 to 10, 1 to 15, 1 to 10, 1 to 8, or 1, 2, 3, 4, 5 or 6;
i) biotin or a biotin analog having the following structure: 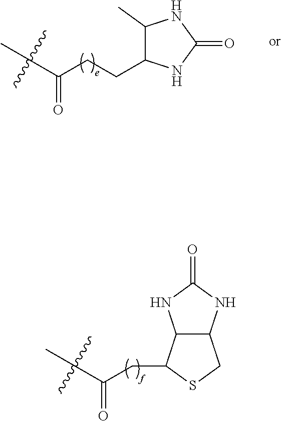
wherein e and f are, independently, an integer from 0 to 10, 1 to 15, 1 to 10, 1 to 8, or 1, 2, 3, 4, 5 or 6;
j) a circular peptide 33 (CP33) or functional variants thereof having the sequence:
VNSCLLLPNLLGCGDD (SEQ ID NO: 1), wherein C4 and C13 form a disulfide bond,
VNSCLLLPNLLGCDGD (SEQ ID NO: 2), wherein C4 and C13 form a disulfide bond,
SEQ ID NO: 3, SEQ ID NO: 4, SEQ ID NO: 7, SEQ ID NO: 8, SEQ ID NO: 9,
SEQ ID NO: 10, SEQ ID NO: 11, or SEQ ID NO: 12,
SEQ ID NO: 3, wherein X is K and wherein C5 and C14 form a disulfide bond,
SEQ ID NO: 4, wherein X is K and wherein C5 and C14 form a disulfide bond,
SEQ ID NO: 7, wherein X is K and wherein C7 and C16 form a disulfide bond,
SEQ ID NO: 8, wherein X is K and wherein C7 and C16 form a disulfide bond,
SEQ ID NO: 9, wherein X is K and wherein C5 and C14 form a disulfide bond,
SEQ ID NO: 10, wherein X is K and wherein C5 and C14 form a disulfide bond,
SEQ ID NO: 11, wherein X1 is K, wherein X18 is K, and wherein C5 and C14 form a disulfide bond, or
SEQ ID NO: 12, wherein X1 is K, wherein X18 is K, and wherein C5 and C14 form a disulfide bond;
k) a synthetic peptide comprising an amino acid sequence of L-Lys-Gly-Gly-L-Ser-Gly-L-Asp-L-Cha-L-Phe-D-Ser-D-Arg-L-Tyr-L-Leu-L-Trp-L-Ser (SEQ ID NO: 13), L-Lys-Gly-Gly-L-Ser-Gly-L-Asp-L-Cha-L-Phe-D-Ser-D-Arg-L-Ala-L-Leu-L-Trp-L-Ser (SEQ ID NO: 14), or functional variants thereof,
l) glutamate urea lysine (GUL); and

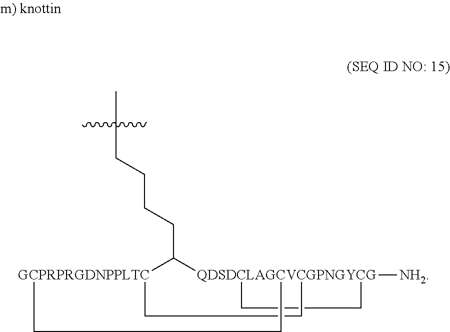

9. The compound of claim 1, wherein L¹, L² and L³, are each independently a direct bond, C₁₋₂₀ alkylene, optionally interrupted by triazolyl, piperdinyl, pyrrolidinyl, triazolyl fused with a 9- to 16-membred heterocyclyl or carbocyclyl ring, and/or one or more heteromoieties such as O, S, S(O), SO₂, OSO₂, SO₂O, OSO₂O, NR⁸, C(O), NHC(O), or C(O)NH, wherein R⁸ is H or C₁₋₄alkyl,

10. The compound of claim 1, wherein at least one of the first and second target proteins is an antibody.

11. The compound of claim 1, wherein at least one of the first and second target proteins is an immune cell receptor, and optionally wherein the immune cell is an engineered T cell.

12. The compound of claim 1, wherein:
the first target protein is an antibody and the second target protein is a cell surface receptor;
the first target protein is an antibody and the second target protein is a tumor antigen;
the first target protein is an antibody and the second target protein is a cell surface receptor; or
the first target protein is an antibody and the second target protein is a tumor antigen.

13. The compound of claim 1, wherein the compound is selected from the compounds of Table 1, or a pharmaceutically acceptable salt and/or solvate thereof.

14. A composition comprising a compound of Formula (I), Formula (II) or Formula (III), or a pharmaceutically acceptable salt and/or solvate thereof, of claim 1 and at least one carrier, diluent and/or excipient.

15. A method for forming ternary protein complexes, either in a biological sample or in a subject, comprising administering an effective amount of a compound Formula (I), Formula (II) or Formula (III), or a pharmaceutically acceptable salt and/or solvate thereof, of claim 1 to the biological sample or subject.

16. A method for recruiting an antibody or an immune cell for immunotherapy, either in a biological sample or in a subject, comprising administering an effective amount of a compound of Formula (I), Formula (II) or Formula (III), or a pharmaceutically acceptable salt and/or solvate thereof, of claim 1 to the biological sample or subject.

17. A method for recruiting an antibody or an immune cell and targeting a cell for provoking an immune response to the cell, either in a biological sample or in a subject, comprising administering an effective amount of a compound of Formula (I), Formula (II) or Formula (III), or a pharmaceutically acceptable salt and/or solvate thereof, of claim 1 to the biological sample or the subject.

18. A method for binding tumor antigens on a cell, either in a biological sample or in a subject, comprising administering an effective amount of a compound of Formula (I), Formula (II) or Formula (III), or a pharmaceutically acceptable salt and/or solvate thereof, of claim 1 to the biological sample or the subject.

19. A method of treating a disease, disorder, or condition that is treatable by engaging an immune response, comprising administering a therapeutically effective amount of a compound of Formula (I), Formula (II) or Formula (III), or a pharmaceutically acceptable salt and/or solvate thereof, of claim 1 to a subject in need thereof.

20. The method of claim 19, wherein the disease, disorder, or condition treatable by engaging an immune response is cancer, or the disease, disorder, or condition is selected from an autoimmune disease, allergy and transplant rejection; and
optionally wherein administering the compound or composition comprises first exposing the compound or composition to the first target protein ex vivo, so that the PBD1 binds its target protein and a covalent bond between the target protein and the compound is formed and then administering the resulting complex to the biological sample or subject, whereby the resulting complex then binds via the PBD2 to its target.


SEQUENCE LISTING (15 sequences)

SEQ ID NO: 1 - protein, 16 aa, synthetic construct
VNSCLLLPNLLGCGDD

SEQ ID NO: 2 - protein, 16 aa, synthetic construct
VNSCLLLPNLLGCDGD

SEQ ID NO: 3 - protein, 18 aa, synthetic construct
  MOD_RES 1: note = X is K, Ac-K(azido), or absent
XVNSCLLLPNLLGCGDDY

SEQ ID NO: 4 - protein, 18 aa, synthetic construct
  MOD_RES 1: note = X is K, Ac-K(azido), or absent
XVNSCLLLPNLLGCDGDY

SEQ ID NO: 5 - protein, 17 aa, synthetic construct
  MOD_RES 1: note = X is K, Ac-K(azido), or absent
XVNSCLLLPNLLYCGDD

SEQ ID NO: 6 - protein, 17 aa, synthetic construct
  MOD_RES 1: note = X is K, Ac-K(azido), or absent
XVNSCLLLPNLLYCDGD

SEQ ID NO: 7 - protein, 19 aa, synthetic construct
  MOD_RES 1: note = X is K, Ac-K(azido), or absent
XGYVNSCLLLPNLLGCGDD

SEQ ID NO: 8 - protein, 19 aa, synthetic construct
  MOD_RES 1: note = X is K, Ac-K(azido), or absent
XGYVNSCLLLPNLLGCDGD

SEQ ID NO: 9 - protein, 17 aa, synthetic construct
  MOD_RES 1: note = X is K or Aryl-SuFEx cap-K(azido)
XVNSCLLLPNLLGCGDD

SEQ ID NO: 10 - protein, 17 aa, synthetic construct
  MOD_RES 1: note = X is K or Aryl-SuFEx cap - K(azido)
XVNSCLLLPNLLGCDGD

SEQ ID NO: 11 - protein, 18 aa, synthetic construct
  MOD_RES 1: note = X1 is K or K(azido)
  MOD_RES 18: note = X18 is K or K-SuFEx
XVNSCLLLPNLLGCGDDX

SEQ ID NO: 12 - protein, 18 aa, synthetic construct
  MOD_RES 1: note = X1 is K or K(azido)
  MOD_RES 18: note = X18 is K or K-SuFEx
XVNSCLLLPNLLGCDGDX

SEQ ID NO: 13 - protein, 13 aa, synthetic construct
KGGSGDFSRYLWS

SEQ ID NO: 14 - protein, 13 aa, synthetic construct
KGGSGDFSRALWS

SEQ ID NO: 15 - protein, 33 aa, synthetic construct
  MOD_RES 15: note = X is K(azido)
GCPRPRGDNPPLTCXQDSDCLAGCVCGPNGYCG